SEQUENCE LISTING (56 sequences)

SEQ ID NO: 1 - DNA, 1327 bp, Arabidopsis thaliana
gagccatatatatgatgctcattgtgtttgttcttatgtaactactcttgcaactctaagttcaaagtgtcaaatcaagattcaagatcatcatcataataaaatatcaaatcacaaacttagaatctcttacacaaacatacaaatagagataacagtaatctttcctcatctattcatcacaaccatatattatccatataataaaaactactaaaaccgaatcgagacaaaaggatcctcatgatctcataatctatagctataacataacatagcaaatatataatcatcataatgactatatattattaagatcaagaatcaagatgtgatcttaattatatcttaacaataagcaatacactccttcttacaatccatagtgaaagtcttaaaaggcttaacaatgattaatgtttgccattttaatctcccttgaccgagttttttcatgttgagtctatatactttaataactaatttatagccaaattaacataatgtggcgaatcatgtaatgtacgtgaaaacgtaattctgttttaagcaaaatttgcacatatacattacgattgtttgatttatcatataatttttgattctgtattttgttaaatagttagttatatattaagcaaagattgcacacattacgattctttgattgccatataattagtttcatcgtactacctttggaatattccactatctatcaaagagattcaactatccgtggtcaccattttataatctataaagtataaagtgtgtaaaaaaaacaaattcaaaacgatatacacattaaaaaaaaatccggaattggtttgctgtcctgtgatcctatatttcggtgtagagtcttctatatttcaaaagttcagaatataatcattctatactaaattgagtaattcagtcaatcatgatctaccaacttcttaattacagttacctaacctactcatttagttagaaattattgatatcctcttatagtcttatactcatttgaattataattaggtaatatatataattaggtacactattcgtatatctataataagaaagacgacaattgtaagagttaaaactgagccaaaaagttatggtgggaatatcagtaacgctacacgagagataaaaccggtctgattcggaattaccataataagttgaataaaccaataattgaatccgaaccaaattcgaatctaaccccaaattttattgcttaagacgaattatttactatttatatgtatataaaaaagcttctataccacacagtcacacatgcacacacttctcacttcagaca

SEQ ID NO: 2 - DNA, 1326 bp, Arabidopsis thaliana
agccatatatatgatgctcattgtgtttgttcttatgtaactactcttgcaactctaagttcaaagtgtcaaatcaagattcaagatcatcatcataataaaatatcaaatcacaaacttagaatctcttacacaaacatacaaatagagataacagtaatctttcctcatctattcatcacaaccatatattatccatataataaaaactactaaaaccgaatcgagacaaaaggatctccatgatctcataatctatagctataacataacatagcaaatatataatcatcataatgactatatattattaagatcaagaatcaagatgtgatcttaattatatcttaacaataagcaatacactccttcttacaatccatagtgaaagtcttaaaaggcttaacaatgattaatgtttgccattttaatctcccttgaccgagttttttcatgttgagtctatatactttaataactaatttatagccaaattaacataatgtggcgaatcatgtaatgtacgtgaaaacgtaattctgttttaagcaaaatttgcacatatacattacgattgtttgatttatcatataatttttgattctgtattttgttaaatagttagttatatattaagcaaagattgcacacattacgattctttgattgccatataattagtttcatcgtactacctttggaatattccactatctatcaaagagattcaactatccgtggtcaccattttataatctataaagtataaagtgtgtaaaaaaaacaaattcaaaacgatatacacattaaaaaaaaatccggaattggtttgctgtcctgtgatcctatatttcggtgtagagtcttctatatttcaaaagttcagaatataatcattctatactaaattgagtaattcagtcaatcatgatctaccaacttcttaattacagttacctaacctactcatttagttagaaattattgatatcctcttatagtcttatactcatttgaattataattaggtaatatatataattaggtacactattcgtatatctataataagaaagacgacaattgtaagagttaaaactgagccaaaaagttatggtgggaatatcagtaacgctacacgagagataaaaccggtctgattcggaattaccataataagttgaataaaccaataattgaatccgaaccaaattcgaatctaaccccaaattttattgcttaagacgaattatttactatttatatgtatataaaaaagcttctataccacacagtcacacacgcacacacttctcacttcagaca

SEQ ID NO: 3 - DNA, 2018 bp, Arabidopsis thaliana
gtagtgaactacgatatatatcattgtggactgacttgtggtgtgtgctgtctcagcgattagcaacctcacaaataaagttaatactaataagtaccctactgtttaacgacctcacaaatcaatactaataacttctaaatttgaaatttgttctctacgtttcacactacatttatggataatcgggtgtatctatagtatatgcatgcgttcgtatgagttttaataccagcgttgactgtcggcaagtaggaaataatccaattaataatacgtttgacaaaagattaaactgtagtactatatataatggaatatttaatccagatatcaaccgttgaaagttatctaatttaatttgataacgatttccaggactgtccccaaatctatctgaaagttattaatcactcctttctaaacaataattgaactttttcttaaaaaaacttctacgacaacacatttcctttgcataacgtagaagtcaatcaaagtttttaaatacttctatcaaatttttaagtaaaatagtattgacacgaaatgcaaaagacgaagtatactgaatataaaatatcacggctacaatgcaacatttaagaattagatgattggaaatcgatacagaaaaataatctaagagaattaggccgtcacttgtgttgtgtgggagcaaaacaaggaccaaaaatatcgggacaaataggttggtccaacctataggtagaggtagcccacttggcatagctcataataccattaccagctcatatgttttttcaaggattggagaaaattaaagaaagatgtaatcgattagagtaacagtggagtgctgaatttaagttagttaagaaaataattggtgttacttcttataaacttttaactcaaaaccaattcgtaatgaatagatagatccatgtctattatatcttatatactattcaaacctcttcttatatatttttccaatgtggattattcgcccatagataaaagataaaacttaacaattggtaagacaatatgacataaagtccttagttctacttacaaagaattttgtcaattaccttccaaaatttagatcttctaaaccctaagttattgggtttcaccaatataatgggtcatttcatctattcacccgaccgttagatttaccaatttctcatcatatctcgattttcaacatttaagaaagtaatcaagtttagccgaaatgcaagatgatacagaaacaatagcgtttaacggtgttagatgataaactcatcaactccattaagaaaaccaatcctgtaagaggtaaagaaggggagaccataattaatgtctaatactttcgtaatgaccactattaatgattagtactatgatctatgaagttgaagctctcttttttttttttttttttcccttcacgtccatagttagttacagcattgatgaaatttttgctgagaatagacgaccctttatcctccaccctacgctttaagtggttgggagttagaccctgccagatagattccaatcctaagataagtctgtttaacaaacctatcatatgtgaaagtgaaaaccattatgttgaagaattatctaaggcgtagagataatttctgcagcaaaaacatttttttaaacattgcgttatacattttaggatagtttatataatcagccaaagtgtatatttctgtaaaacacattactatcttgacatttttgtgataagctatataatcagtaacctgctacgtatagcttaaccccactattataattatgattcctcattcagtaaaactatatagctgaattaataaagtttattagggtctaatgaagttggtgtgatcatttaataatattgttatttcataactcggaattgaattatttattacccttgccatcttaaatctacatttgcaactcacccaaaagctttatcctttgtgttttttccactgtatactgaaaacaaatctgaggtgacgaag

SEQ ID NO: 4 - DNA, 1974 bp, Arabidopsis thaliana
atacaaaaatattttatagtagtgaactacgatatatatcattgtggactgacttgtggtgtgtgctgtctcagcgattagcaacctcacaaataaagttaatactaataagtaccctactgtttaacgacctcacaaatcaatactaataacttctaaatttgaaatttgttctctacgtttcacactacatttatggataatcgggtgtatctatagtatatgcatgcgttcgtatgagttttaataccagcgttgactgtcggcaagtaggaaataatccaattaataatacgtttgacaaaagattaaactgtagtactatatataatggaatatttaatccagatatcaaccgttgaaagttatctaatttaatttgataacgatttccaggactgtccccaaatctatctgaaagttattaatcactcctttctaaacaataattgaactttttcttaaaaaaacttctacgacaacacatttcctttgcataacgtagaagtcaatcaaagtttttaaatacttctatcaaatttttaagtaaaatagtattgacacgaaatgcaaaagacgaagtatactgaatataaaatatcacggctacaatgcaacatttaagaattagatgattggaaatcgatacagaaaaataatctaagagaattaggccgtcacttgtgttgtgtgggagcaaaacaaggaccaaaaatatcgggacaaataggttggtccaacctataggtagaggtagcccacttggcatagctcataataccattaccagctcatatgttttttcaaggattggagaaaattaaagaaagatgtaatcgattagagtaacagtggagtgctgaatttaagttagttaagaaaataattggtgttacttcttataaacttttaactcaaaaccaattcgtaatgaatagatagatccatgtctattatatcttatatactattcaaacctcttcttatatatttttccaatgtggattattcgcccatagataaaagataaaacttaacaattggtaagacaatatgacataaagtccttagttctacttacaaagaattttgtcaattaccttccaaaatttagatcttctaaaccctaagttattgggtttcaccaatataatgggtcatttcatctattcacccgaccgttagatttaccaatttctcatcatatctcgattttcaacatttaagaaagtaatcaagtttagccgaaatgcaagatgatacagaaacaatagcgtttaacggtgttagatgataaactcatcaactccattaagaaaaccaatcctgtaagaggtaaagaaggggagaccataattaatgtctaatactttcgtaatgaccactattaatgattagtactatgatctatgaagttgaagctctctttttttttttttttttttcccttcacgtccatagttagttacagcattgatgaaatttttgctgagaatagacgaccctttatcctccaccctacgctttaagtggttgggagttagaccctgccagatagattccaatcctaagataagtctgtttaacaaacctatcatatgtgaaagtgaaaaccattatgttgaagaattatctaaggcgtagagataatttctgcagcaaaaacatttttttaaacattgcgttatacattttaggatagtttatataatcagccaaagtgtatatttctgtaaaacacattactatcttgacatttttgtgataagctatataatcagtaacctgctacgtatagcttaaccccactattataattatgattcctcattcagtaaaactatatagctgaattaataaagtttattagggtctaatgaagttggtgtgatcatttaataatattgttatttcataactcggaattgaattatttattacccttgccatcttaaatctacatttgcaactcac

SEQ ID NO: 5 - DNA, 490 bp, Arabidopsis thaliana
tcatgacagggtaggattttatttcctgcactttctttagatcttttgtttgtgttatcttgaataaaaattgttgggttttgtttccttcagtggtttgattttggacttatttgtgttaatgttgttttggctgttctcttaatatcaataacaaataaatttactggttggtatctaagatctaacaatagttactatttttagaggtaaagacaccaaccttgttatattggtcagagagctaaaaccttgacttgttgggaaaacaaaactctaatgacagaaaatctgacatgatgccttataattcacagcctcatgttctacataaatcctaacaatagcactttgtttcttcattatattttgttaagtccactcttctctctcatatcttctaaccaaaacagagtcacaaggggctcttaagcccttccaactaaattcttttcttttgttctcttgaaactgaatccaccagacaaaa

SEQ ID NO: 6 - DNA, 2255 bp, Arabidopsis thaliana
tgggttttatttttgacatttggttttatactttagttccgttgactttcgcctccaccataatttctccaattcagatttgattcggtctgaacacaaagtccggtttggtttcttatttgtcttaatatcgattactttccatctataaaatatttttctacaacatcttaagaattataattgagtgatgttgatgctactattttaagtttagaaaataaacactaaaaagacaaatgtctcactcatcaaagtaaaactcttgaaaagtgcaagagctctgaaatttgagaacgaagacaagactccttgtttttttttgtttttttttgctaaaaatttaaatattcattattacaatgaaaatttcggttacataataaatggtaaccaaatcatggttccatgacaaaaaaggataaaaagcatggaagcataccaagactccttgttactacgtcaatctcttttatacgttttcagccaagattccggattatgaaagaatcttgggattctaacactttttctttttttgcttgaaagaggtttacaaattttaacactttttttttgttgaggattttagagtgaaacacatgttttgaactgtcttcaactgaacaattcatgttaggcgtctatataaccgtcggttattcacgaggtaactacacatgaacatgataaatttactctctcttttcattaaaaaaaagttgtacaacttaattacttatgtcatgaaaatagtatatacgtaaaagtagattatttttgtggttttccttttttttactataacaataaataattctatgttacctaaattttcttaggtagtataatggatcaaattgatatggagtaaacaaaagaaaaacttaaataatctggtctataatttgaagcgcttcaagccttcaacatcaatccgagtacgaacaataatatgagatttcatcaaaatattatcctggaaacgatttttcatttatatgcgattatattgttaatgaaagttggaaatacataatctagacacgtaaatgtcgtattgatcatgttgtgaaatgagctgtcgccttggtggcactttttggcattctctatttctctttccacatttaccacaatgtatccaaataggcaaatatataagcttagagagttggctgcacgtttttgctaaacttgataaatgagtcaatacaaccaatatagccaccatccatatctacaaatctacacttatcatctaaacttgaagaatatttgttattttatcactaaccacaaaagacaagactcgttacttaagttaaatgatagtgacatgattaagagaatattagctattaggtcggaaataagagaaataagactggtagtggtatggttatgtaaattatcagtacatgtatataacacttgtccaaataatggctttcacattacaagtcattctttccctgagactactgcaagaaacaaacacggaattctcgtgataaacggattagtacgaaggaaaaagtaaaatgcagtaaccaatttttatatttcaaaaaacaaggcattttggatgcaatgaaatatttagatatataaatttgactagtgacaacaatttaaagttgttagatttctcaaatccaaaaaaaaggaaataaataaataaatagtttatggctattcaaattgtgtattattttttctattggttaaaatctataaaagattttttttttattacttcttaaatttatgtttatagccaaaacatctaataaaatgggacagagaataataactaggaattcaaacacattatcaatgattagcagaataaaagtttggaacatctaaacctaatgactttatacttcccctttttagagtttactttgtatggaaaactttgtaagctaacaaacaaaagtattgaaatcgtgaaaaatagtaaagctttttgagctgcaatatttgatgcgttgaaacgagttggaaacagctttcactacactaaaaacaaacttaatctcaaaatttagatggattaaactcaaaactttttaattaattgaataggattttaggatgatgcagtgaatatagactatttggtgaaaaaatacaacgtaacgtacgtggctgctctaagcctatataacatagcccaagagagtcgtgttctaatgtgattaagtaaagtgagggagaagcaacgagagatagagatagagagatca

SEQ ID NO: 7 - DNA, 1185 bp, Arabidopsis thaliana
ttctctctagcaaaactctctctctttctcccttgtagaattaattagctatcataaatatagtagttcatcagttccacttccactaaattattgtttttggcaaaacagtaacttaagttatataaaaaaaaaaatcattagtcaatcaatcacagtcctttatgataaaacgaactcataattattccaccgacaacatgcgttttaaattattttttcttaaattatattatattatattgatatcaacctagctaaaataattcggatggcgaaatcggacaatttttaatagaaaaaatgggtatgaagatagtctatgattccgttcttagcgactagagggacctgctcaaatctcccgggtgatacgcgatgtcaagctcaatagaaccccacaaccgacgagaccgagaaatccttgatttgggctagaagattttgaaatgaatttaatatattctaagtaacttgcttaaattttttttcaaactctaaagacataactaacataaagtaaaaaaaaaaagttaatacatgggaagaaaaaaattaaactaatgattagctctctaacgtgtttaatctcgtatcaagtttttttttaaaaattatattgctattaaaacattgtactattgtttctattttgtttagctattattcttgtgaaatgaaaagttgtgtttattcaattactaaatggcaatatttatcttggaaaactatacctctaattggattaggccctagacatcctctttagcttattgacgttaaaattattcccaaaactattaaagtttagtagtttgaaagatgcatcaagacctactcagataggtaaaagtagaaaactacagttagtgtgattatattttaaaatatataaaacaatcttattaaactaaatattcaagatatatactcaaatggaagataaaaacatttagtctgttaccactaccagcctagctagtcactaatagtcactttggaactgagtagatatttgcatcttgagttaccatggactcaaaagtccaaaaagagaccccgagtgaaaatgctaccaacttaataacaaagaagcatttacagcggtcaaaaagtatctataaatgtttacacaacagtagtcataagcactcaacacaaactctttacgaatacttttaaggc

SEQ ID NO: 8 - DNA, 2119 bp, Arabidopsis thaliana
cagaatatctaaccatttcatccagattatatatttgttaatatctaacattatcgatattctatcgcaacatggaatcattaatatctaacaatttcgaacattttcaatgttcataacgcaaaacaatgtcaaagtaaattcaaactacacgaagtaaatgtattgtatgaccacatatacaaagtataggacgtcatgtggttaacaccatagacatacaattccgataaaccggtcagttgactccggcgttgactagggttgaccggcgttgaccaacaaaaaaattcaaaaaaatcttttaaattattttaaatattcaaaaatacaaaatattttttttttggttttgtatattcaaaaacatattctatatttcaatgcattaaatcttagaaaaattagttttacaaaaaaaatcaaaatttaactaaaaatagattaaaaatcattattaaattttaaattttaaatgaaaacaggaaaatattattatagttaattaagtaaggaaattgcttatttttatagtgtcaattaaaacacttcaattatttctatacaatattttttataaaaaaaaatcaaccacaaaaattattagaataaaacgtaatacaaatgaattttattttaaaaacttttttgctgaaatcaacattgttagattttctatctttttatatattaaaaagaaaaattgcaagtttttggttgtttatgtgttactacgagaacttttcttaataatatttgttacaaaaggaactacatagtatacaaaaataaatttagactaaagagtatataaaaaatattataattttctttaccatgcaaactttagattaaagagtcatatactcaatttcatattgcttcctaatacaattgagtatatgactctttaatctaaagtttaataatgatttttattctagttttagtttagttttgaaattaaaaataaaactaattattataagatttaatgcattgaaaatacaaatatatttttacgaaatatagaatatgtttttgaatatataaaagaaaaaaaatattttcgtatttttgagtattaaaaataattttaaatttttttgttggtcaacgccggtcaacactagtcaaagcctgagtcaactgaccggtttaccggaattgtatgtcaatggtgttaaccacatgacgtcctatacttcatatatgtggtcatgtaatacatctacttcgtgtctacttcgtgtagctggatatacaatgtatagtaggtatgtgtgaccatgtattctcttatactttgtttacctagcaatcttttttttaaattaaaataaatatgcggtttagatatgaaactacccaacaaatttaacattttaaacgttcataacgtaaaacgacgtcgttatagacacatattttccatgtgtctgctgacttatcatcttcacggagttgactaacacccgttactttgactctgaattttgtactttttcttaagttgaggtatgaaattcaaataaatatgcggttaatatatgaaaatacccaacaaatttttttggatacgaaaatacactcagaaaatagtacgggtatgaaaatacccttttcccgtatttgatacatgtctaattcggttcaaataaaccgaatatgaaaattttcagttttatttcggaagttaaataaatctagataaccgacctgaaaaacccgagtcccgaccgaaccgaaccgaaattaaattcggtttaattcggaagcatttccaaaaaccgaaattccctaaaaccgaataacccgacccgattaaaccgatttgccgaactcccaggcctaaattcacacttggcttagaaaaactctttgtagatgttaaaattcggtaaaattaacctcaccaaagctaattattaccaggtgaagaaagcattaaaatttcaaagtgtgtatgacagaggttttagaaagcgactgatgtacggacatatcaacaactcccctataaagatactcagctaaacacaaaaacagaatctattctcaacacaacactaaagacaattgtaccaaccacacaaccacaagagagagaaaagtgacc

SEQ ID NO: 9 - DNA, 853 bp, Arabidopsis thaliana
tggttctgctacatgcagatgatactatccgttgttgaatttgtcgattagaattctttttggtgtacacaatgcggttgtcataacgccttaatagcttgtattagtcaaagaactgcatatggtcttgtgttttcttgtcatcgtgtttttgtaaccacaaactgttttgagctatactactatatatattgagatatatctgccgtttcgatacacacttgggatctggggatgagcacatcgtaaaacaaaatagaagttgatcctcaaaacttctttgtaaccttgtgtcatcacaacaaaaaatcttcaatgtgtttgttctctccttaaagtatatcttgattcatgcagtaacaaaggcaaaactcttttgcaagagtatagaaaccagactcaagctgtgcgatggtgattcttttggagaagttggatttgtgctctgatgtaaagggaaacttaagctaaaaggtccatcaatggaggtgacacatagttttagaaaatgtgcttttctcatgctagaaatgttatggagacccaaaaatgcttttcggaaaaaattctcatgctagtagctaggctctacttaacgaggtgacagctaaaataagttctttttattccattttcagaatagtgacattcttctcacaaatatagaaaaactacaattaatgctactgcagagtctgattacgttttaagctaatttttccatttttaggacgtggtagattgtgtagattattgctaaacagctcatgagttcaataattcacttattcttcactccatcttcagcaaaaaaaaaaaaagtaagaagaaacactgaaagctctccactacc

SEQ ID NO: 10 - DNA, 4755 bp, Arabidopsis thaliana
aattcgatagacgctgggtaaaaaaattcggaggacgacgaaagagaaaacgagtgtttcagtcactgccccacggagctctcggaaatttgtcttccccttgtcgtcgtctccctatctactgcttcttcttcgttttcgtcttctttatcaaggtgcgctttagcttctcaacgccgtttgatttttagaatttcgatttttttttttttttcttctagttcttgaatcaatccggaatttggcgactatgttgcttcgtttgtaaatcgtattctcctgtttagaaatcttcaattgactgtgttataggaacaatttaaatctcaatttcaatgtctcttttagtcaccttcgtgtagtaatttgcttttgaattactgttaatgaatctcaaaaaatggattttataatttgggaaaaggggcttctgggtttaattaaagaacacgagataaggtctggttttttcttttcatttctttgtgtgtgtttttggtttctttgattttcttctgggttatggtccgtttgagtctggtgatagttagttggcaaccaatttttattgatctattacaatcgagaacacaaaactaaaccctaagaaagaagtacataaagttgttgaaaagatctcgttaactctcccaaagtcctagggctttcacacaaccagtgattaaataacctttgagctgttctccttcccacactttatatgtgtgtttgtggtttgtctaatttgtgaggagcttctatgaaacctctggttattttaattgttttctgcaattcctgattgatatgtttatatatatttcttgtatttgtgaatttgtgtaggaatgctgtttaattggaatcaacaatggagaatttgacggaaatagaatcaacgatggagagtttaacggaaatggagagtgagagagttgaacagggtaccgataaggaaattggaagtggagagaaaaggcaggatgatgtaaaggaaacggagaatgagaattctggagagagagtaggagaggaagctcctgtcagggaacatgaagattctccatgtctcattgttattgaagaaggtacttccctagcttcccttgaggaggtgaccaatgctgatgatctgccgaagattgatgatgagaagaattcccaatttgaaacaagcccgcatccaagtccttctccttcagtagctttagacactgaagaagggttaatcaaccctactgcagaagacactgtagaagagaacatagtgtctagcgaagtaagttcggatatcttgaaagatgacggagatgccgtcgaggttgacagagatactgcagaagtccaggaagaaacggccaacatacctgaatccaaactctcggaggacacaggatcacctcatcatcatgctgatattctgatggtgcaggaaaaagctgcagaagaacatgacatgatagcctctggagaccatgaagaatttccagtcaatcctgataacaaacactctgaagaaaatcagtcaccacatcatcatgctaataatgtgatggagcaggaccaagctgcagaagaacgtgagatcatatccccaggagaacataaggaaattccagccaatcctgatactaaagttgttgaggagaacaatgacaggatagatgagggtgaggctaacaatttgaatttggctggcgatggaagtggagcagtcgatcatgattacttgaccaaaacggagctggacaaagtgctagaggtgcctggttctgagaccatatcaaaactggaggataggccatctgagcatctctcagaaacctcaatgaacgtggaaaaagaactagaaatgcctgccgttgaaattttgccagacaatgacaaaaactctgatgtgttggcagttggagtttctggagacagtgacaatgtggtatctgtcttgcccgcttcccaaacttcctctgatcgtgatgaaggaatgattacagttgatgctgaacctacggaagacatgaaacttgatgttccagattctaaattggttactgatactactgttgactctactaataacaaggatgcccatgttgaggctaatactgaaaggcaagataattctagtgcacttgtgctaaatgatgcaaataatgaaagtgcaccagtgaaacgtgtacctggtccttatgttgcatcttccaatataaagtctgaagcgcggggtagtggagatttgaacaatggagtacataaaatagttcggaccccacctgtctttgatgggaccatgcgcgcaaagcgctctttcctcttggatgatgcgtctgatggtaatgaatctggaacggaagaggatcaatctgcttttatgaaagaattggatagtttttttagagagcgaaacatggatttcaaacctccaaaattttacggggagggactgaactgcctcaagtaagcttgatacccatcattatttggtcactttactgtgttacattttaaaattttcagcaggagctgatatctaatcaatttctttggcacaaggttgtggagagctgtaactagattgggcggatatgacaaggtacgggtcactgtgaatacgcctgttgaatgtcacagcatcttttttgacaagcaaatgtgacttcggcttttcatcttttgttccatcctggcttacttgcatgcgtactgttgttcatgatctagcagtggtgcttttggtgattttctatgattattatatgctttttatactggataggttactggaagcaaattatggcggcaagtgggagagtctttcaggcccccaaagtaagaagaatgcttttcttattagtggtttgtcttagaaattttgggaaatcatgtggatatttttaagaattaccctctaattggtcaattgtttgttcaggacatgtacaacagtatcatggactttccgaggtttctacgaaaaggtgagactatattcaccaccttttcctctctctgcttttggttcgtctatgtgacttttgtatacactggcatgggactgggactctatgtatcaacccttctgagaaataattgaaatgattgaacagtgaacaactgtgaatcatcttgagatatgttttccttaagatacagtaacatcttgtaacattatagtttcttcatttttcaggctcttcttgaatatgagcggcataaagttagtgaaggtgaacttcagataccccttccgttggaactagaaccgatgaatattgataatcaggtaaaattgagaaaaccatatcatgtgtctgtagtttttgtttgatcttcttcttctgattaatgtcagtgttttaacttaacccactgccttgtttctacactaggcgtctggatcagggagagcaaggagagatgcagcatcacgtgctatgcaaggttggcattcacagcgtcttaatggtaacggtgaagttagtgaccctgcaatcaaggtccggtagaatctttttatatgtttcattttacattcacactagatctctcgttttttttttgtcaaacatttaatctatatctcatagtctgaacgaacatactgttttgtaattaataggataagaacttagttcttcatcaaaagcgcgaaaaacagattggaaccacccctggtatgagttctgtttgatgaagaagtgttgttctcatttttattttgaaactttgacatgggttatcacttacatctcacaatgtcatcaggtttgctcaaacgtaagagggctgctgaacatggtgcaaaaaatgccatccatgtatctaaatctatgtacgatttttggctttgtggtctggttttcaatgcgtgataattcacatttgaattctgattccagttgttgtttttcctaggttggatgtgactgttgttgatgttggaccaccagctgactgggtgaagattaacgtacagagaacggtaaaatcaattgccactttcttaaaaacctgagcaatcactttctggttttacatatattaataaactcttccactatctgcagcaagattgctttgaggtgtatgcattagtcccaggattagtccgtgaagaggtaagctctcaaatctcgttgtgtttacatatggatcctaagattgagtttagcactcagtttttgtcttggcaacaataatacaggtccgagtccaatcagatccggctgggcggttagtaataagtggcgaacccgagaaccctatgaatccttggggagctactcctttcaaaaaggtaaatgctggttacatgatttttcagcttacacgtagaatgttgaatgacattttcaaacctccattgaaactgcaggtggtaagtttaccaacgagaatcgatccgcatcacacatcggctgtggtaaccctaaacgggcagttatttgttcgtgtgcctctggagcaattggagtagaaacatttacagtttaacaaagcctttgaagatctgaaagagagaagattgttagaagtagttgttgagagtattttgtttgtatattatgagagattaagcacaacatgagaagagcctttaggaatccttaattaggccatctagtttttattgtctctcctctctttgattagattcttcttctaagtgtcatcactattgatttgttgtagcaccaaacttctttaaacctttctattaagaacacacaaatctacaacctttttatttttttt

SEQ ID NO: 11 - DNA, 810 bp, Arabidopsis thaliana
atgaaatcgttttgcaagttggagtatgatcaagtgtttggcaaagaaaataattcattctcatttctaaaccactcatcactttactctcatcaaagcgagttagcaaatcctttcttcgagttggaagacgagatgcttccttctgctacctctagtaattgttttacttctgcctcaagctttctggctttacctgatcttgaacccatctccattgtgtctcatgaagcagatatacttagtgtgtatggttctgcttcatggaccgcagaagagacgatgttcgtttctgattttgcgaaaaagagtgaaaccacaactaccaagaagaggagatgcagagaagaatgtttttctagttgttctgtttcaaagacattgtcgaaggaaaccatctcattgtacttttacatgccgataactcaagcggctagagagcttaacattggtttaactcttttgaagaagagatgccgcgaattgggtattaaacgttggcctcatcgtaagctcatgagcctacaaaaactcatcagcaatgtcaaggagctagagaagatggaaggggaagaaaatgaagataagctaagaaacgctttggaaaagctcgagaaggagaagaaaacgattgagaagttaccagatttgaagtttgaggataagacaaagagattgagacaagcttgtttcaaggctaaccataagaggaagagaagaagtggcatgtccacgcccatcacatcatcatcttcttctgcttctgcttcttcttcttcttactcttctgtttcgggttttgagagataa

SEQ ID NO: 12 - protein, 269 aa, Arabidopsis thaliana
MKSFCKLEYDQVFGKENNSFSFLNHSSLYSHQSELANPFFELEDEMLPSATSSNCFTSASSFLALPDLEPISIVSHEADILSVYGSASWTAEETMFVSDFAKKSETTTTKKRRCREECFSSCSVSKTLSKETISLYFYMPITQAARELNIGLTLLKKRCRELGIKRWPHRKLMSLQKLISNVKELEKMEGEENEDKLRNALEKLEKEKKTIEKLPDLKFEDKTKRLRQACFKANHKRKRRSGMSTPITSSSSSASASSSSYSSVSGFER

SEQ ID NO: 13 - DNA, 897 bp, Arabidopsis thaliana
atggctgatcacacaaccaaagaacagaagtcattctcattcctagctcattctccatcctttgatcacagctccttaagttatcctttattcgactgggaagaagatcttcttgctctccaagaaaactctggctctcaagcatttccttttactacaacttctctgcctttacctgatcttgaacccttgtctgaagatgtactcaattcatacagctctgcgtcatggaacgaaacagagcaaaacagaggagatggcgcttcatcggagaagaagagggaaaatggaacagtgaaagagacaactaagaagaggaaaatcaatgagagacacagagaacatagcgtgagaatcatcagcgatattactacctacacaactagttcagctccaacgacattgtcaaaggaaactgtctctcgctacttctacatgcccataactcaggctgcaatagcacttaacgttggtttaactctactaaaaaggagatgtcgcgaattgggtattcgccgatggcctcatcgtaaacttatgagcttaaacactttgatcagtaacgtcaaggagctgcagaagatggaaggcgaagagaatgcagaaaaactgcaggacgcgttggagatgcttgagaaggagaagaggacaattgaggatttgccggatttggagtttaaggacaagacaaagaggctaagacaagcttgtttcaaggctaaccacaagaggaagaagaagagaagtctcaagtccgatcagtctcaagtaccctcgtgttcaagcagcggatcagttcctagtgatgagtcggttgatgaagcaggaatggagagtgatgaagaaatgaagtatctcttgtgtggtttctcaagtgaatttactagtggtttgtga

SEQ ID NO: 14 - protein, 298 aa, Arabidopsis thaliana
MADHTTKEQKSFSFLAHSPSFDHSSLSYPLFDWEEDLLALQENSGSQAFPFTTTSLPLPDLEPLSEDVLNSYSSASWNETEQNRGDGASSEKKRENGTVKETTKKRKINERHREHSVRIISDITTYTTSSAPTTLSKETVSRYFYMPITQAAIALNVGLTLLKRRCRELGIRRWPHRKLMSLNTLISNVKELQKMEGEENAEKLQDALEMLEKEKRTIEDLPDLEFKDKTKRLRQACFKANHKRKKKRSLKSDQSQVPSCSSSGSVPSDESVDEAGMESDEEMKYLLCGFSSEFTSGL

SEQ ID NO: 15 - DNA, 834 bp, Arabidopsis thaliana
atggctgatcaaagacctctaatgacctggttagaggccaacaactatgaatcattccttcaagaagacatattctcgtttctcgatcaatcacttttcgtcgatcctcacagctctttcattgacccttttaaggattttcaaacccaaaattggttttctctccaagacagcattgttaatcatatatctactacctttgcggctgatcatacgtttctggcttcacttgatcttgaagctatctctagtactttctctctagatatatcgagtggatggtggaacgagaataatggtaactacaataaccaggtcgaaccaaaccttgatgaaatttcaagaactaataccatgggagatccaaatatggagcaaatattgcatgaagatgttaacacaatgaaagagaaaacaagccagaagaggataattatgaagaggcgatatagagaagatggagtcatcaataatatgtcaagggaaatgatgaagcagtacttctacatgccgataactaaagcagccaaggagcttaacattggtgtaaccctcttgaagaaaagatgtcgtgagttaggtattcctcgttggcctcaccgtaagctcacgagcctaaacgctctaattgctaatctcaaggacttgttagggaacacgaaggggagaacgcccaagagtaagctgaggaacgctttggagcttttggagatggagaagaagatgattgaggaagttcccgatttggaatttggggataagactaagaggttaagacaggcttgcttcaaggctaaatacaaacggagaaggctcttctcatcttcttcatga

SEQ ID NO: 16 - protein, 277 aa, Arabidopsis thaliana
MADQRPLMTWLEANNYESFLQEDIFSFLDQSLFVDPHSSFIDPFKDFQTQNWFSLQDSIVNHISTTFAADHTFLASLDLEAISSTFSLDISSGWWNENNGNYNNQVEPNLDEISRTNTMGDPNMEQILHEDVNTMKEKTSQKRIIMKRRYREDGVINNMSREMMKQYFYMPITKAAKELNIGVTLLKKRCRELGIPRWPHRKLTSLNALIANLKDLLGNTKGRTPKSKLRNALELLEMEKKMIEEVPDLEFGDKTKRLRQACFKAKYKRRRLFSSSS

SEQ ID NO: 17 - DNA, 771 bp, Arabidopsis thaliana
atgagttcgtcaaaacattcctctgtttttaactattctgctctgtttctatcactgtttcttcaacaaatggatcagaactctcttcatcatctcgattctccaaaaatcgaaaacgagtatgaaccagattcgttatacgacatgttagataagttgcctccgcttgattctctcctagatatggaagatttgaaaccaaatgcagggttgcactttcagttccattacaatagctttgaagatttcttcgaaaacattgaagtggataacacaattccatctgatattcacttgttgacacaagagccctacttctcaagtgactcctcttcctcttcaccattggctatccaaaacgacggtctcatttccaacgtgaaagttgaaaaggtaacagttaagaagaagaggaaccttaagaaaaagaggcaagacaaattggagatgtctgagatcaaacaatttttcgataggccgatcatgaaagcggctaaagaactgaacgtgggactcactgtgttgaagaagcgatgcagggaattaggaatttaccggtggcctcaccggaagctcaagagtctaaactctcttataaagaatctcaagaatgttggaatggaagaggaagtgaagaacttggaggaacataggtttcttattgaacaagaacctgatgcagaactcagtgatggaaccaagaagctaaggcaagcttgtttcaaagccaattataagagaagaaaatcacttggtgatgattattattga

SEQ ID NO: 18 - protein, 256 aa, Arabidopsis thaliana
MSSSKHSSVFNYSALFLSLFLQQMDQNSLHHLDSPKIENEYEPDSLYDMLDKLPPLDSLLDMEDLKPNAGLHFQFHYNSFEDFFENIEVDNTIPSDIHLLTQEPYFSSDSSSSSPLAIQNDGLISNVKVEKVTVKKKRNLKKKRQDKLEMSEIKQFFDRPIMKAAKELNVGLTVLKKRCRELGIYRWPHRKLKSLNSLIKNLKNVGMEEEVKNLEEHRFLIEQEPDAELSDGTKKLRQACFKANYKRRKSLGDDYY

SEQ ID NO: 19 - DNA, 360 bp, Artificial sequence
  EASE promoter
ccacgatgcaaatatatcgataacgttattaaaaaaagtaaccgcatgatatattctctttcgtatgatattaaggcccacgatgcaaatatatcgataacgttattaaaaaaagtaaccgcatgatatattctctttcgtatgatattaaggcccacgatgcaaatatatcgataacgttattaaaaaaagtaaccgcatgatatattctctttcgtatgatattaaggcccacgatgcaaatatatcgataacgttattaaaaaaagtaaccgcatgatatattctctttcgtatgatattaaggcgatatccaagacccttcctctatataaggaagttcatttcatttggagagga

SEQ ID NO: 20 - DNA, 520 bp, Arabidopsis thaliana
gaatttaactgatttggtcatctttaagatcataagtattaataaggaatccaaaagttatttaaggttttgttagaaaagcaagataggcatcatgagttagtatctatatataatatagaactttttgatctttttaatcaaactatattatacatatgtcttagttcctaataaaatgtgggcttcaatagaatttttgaaatataaagttttaaacctgtaattgtttgcacttattagatgtatattactatttataccaatatataacagattttaataactaaacaattataattttttaacaaaaagcaaacgtaataggttactgaattttactttataacaaaataaaacgtttaaatgaaaattaactctttatataacatatttatctacagagcctataaatatgactaaatattgctttaatactccagagcaaaacaaaagaaaaacaattcacaataatatttaatatattttctttgtgatattggttaatttctaccaagaa

SEQ ID NO: 21 - DNA, 1419 bp, Arabidopsis thaliana
tggcagggatacccagaaaccacatttgcttacatgtcttctctataacagagtgtgtaaagttttgtgtgttgaaaggtttttaattttaagcaaaagtggattatgacgacaacagacaagcttttaattttattttaccgtaatagttatatcttgttgtaagaaaccattttcagccttttgttggaaaatcctgcttaaatggtttttgagtcttacataatagcttcttcatcttttgtcttcttaaagagaattatatttgtaatttcatgtctgttgtgtttctttgactttactgaatagagaatttgtgtgtttatggtgaaaatatagccgatctgcttgacagatgaacggagtttattttgtctggtgacatgactctgttctcttatatcaggatttttgagaaaccctttggtatctttattgtttggtctgaaggtatgtatatactttttgtctttgattaacctagtaatatgattactaactcctgtaagttcctctttcagatcactagaacaaagcaagaagttgtaatatctattgtatagtataaagatgctcgaaaaatttcagattctggttagctctagttgtacagaagaacaaaaaagtctctaaagactcaaatgtttcagaacgacctacgcctatgagtgtctaaaccggttaaatccgaaccgaaacgaatggaaacagtcttgagaaacaaaagagtaaaaaactgatcatagaatcacctagttttactaaaaagtggtatttaataaaattgctctctaaacaactttattaataacctacaacaagatttaatttctcatttcttaagaggccattaactacaagaatcacctgaaaagtattaactactcgcagccattatctccaattaattgaaaccgttttttttttggtgggaaatgtattattaatttcttaaccgttactcgcagctccaactataagtttataactatttttcgttaacaattaaaatattaattggcaccatacgttacaagttaactgattacaaatactaaggagtatataatacttaggaaaaactgtaattatatgaaatcaactagctacttcacaaaagagcaaattaactacgattggcttataaattatatccatagatcagagagatgctaagagagacgtctatccattacctaatccttaaaaaaaacgtccctcttattagcattagttaccattaatcatttatatctctctcgtaactccaaagtttttacagggcaatcaattagccgtcatacccactttcccgtacattttataacttcacttctatatctaccactacatgcatgtatatatatatacacaccgttctctctctcccgttgattagtgatcacaaacccattaata

SEQ ID NO: 22 - DNA, 579 bp, Arabidopsis thaliana
aaatctttggctttttggatcgttcttttgtggaaatggaatataaaacttttttgttacttcattaataacttatgattaattatgagaaatggaaattaaagatatatggccatgatctacaataatgttttaaccatacgtttcattttgttatcttaatcattcagttagtggttattaaacaatacataatcatgatcattgtgatgtgtatgtatgcgtatatataagaacatgtacattgagtagtactacactatttactcgaaatgattgcatgtcatatatgcatggagagacgaaaagaggagtctaatccaaatctaaacgcccctataaattacccactaattaacattaatcatatcttctcgtaactccaaatttaacacgacaatcaattagccgtcaatactcaataccccacttctcctaatagattcatcatcacttccattctttattctctctccatatcttactaccactagtctcttctctgaatgtagtatataaatcttttctcgcatcatcgagtttcacaacacaacttctatctctctcactttctttaca

SEQ ID NO: 23 - DNA, 525 bp, Artificial sequence
  intein
atggcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggggcaaaagcggacgaacatggcgtgaagcggatattaactatacatcaggcttcagaaattcagaccggattctttactcaagcgactggctgatttacaaaacaacggaccattatcagacctttacaaaaatcagataa

SEQ ID NO: 24 - DNA, 846 bp, Escherichia coli
atggggacaatgaagaaaaatcgcgcttttttgaagtgggcagggggcaagtatcccctgcttgatgatattaaacggcatttgcccaagggcgaatgtctggttgagccttttgtaggtgccgggtcggtgtttctcaacaccgacttttctcgttatatccttgccgatatcaatagcgacctgatcagtctctataacattgtgaagatgcgtactgatgagtacgtacaggccgcacgcgagctgtttgttcccgaaacaaattgcgccgaggtttactatcagttccgcgaagagttcaacaaaagccaggatccgttccgtcgggcggtactgtttttatatttgaaccgctacggttacaacggcctgtgtcgttacaatctgcgcggtgagtttaacgtgccgttcggccgctacaaaaaaccctatttcccggaagcagagttgtatcacttcgctgaaaaagcgcagaatgcctttttctattgtgagtcttacgccgatagcatggcgcgcgcagatgatgcatccgtcgtctattgcgatccgccttatgcaccgctgtctgcgaccgccaactttacggcgtatcacacaaacagttttacgcttgaacaacaagcgcatctggcggagatcgccgaaggtctggttgagcgccatattccagtgctgatctccaatcacgatacgatgttaacgcgtgagtggtatcagcgcgcaaaattgcatgtcgtcaaagttcgacgcagtataagcagcaacggcggcacacgtaaaaaggtggacgaactgctggctttgtacaaaccaggagtcgtttcacccgcgaaaaaataa

SEQ ID NO: 25 - DNA, 375 bp, Escherichia coli
atggggacaatgaagaaaaatcgcgcttttttgaagtgggcagggggcaagtatcccctgcttgatgatattaaacggcatttgcccaagggcgaatgtctggttgagccttttgtaggtgccgggtcggtgtttctcaacaccgacttttctcgttatatccttgccgatatcaatagcgacctgatcagtctctataacattgtgaagatgcgtactgatgagtacgtacaggccgcacgcgagctgtttgttcccgaaacaaattgcgccgaggtttactatcagttccgcgaagagttcaacaaaagccaggatccgttccgtcgggcggtactgtttttatatttgaaccgctacggttacaacggcctg

SEQ ID NO: 26 - DNA, 372 bp, Artificial Sequence
  intein
tgcctttctttcggaactgagatccttaccgttgagtacggaccacttcctattggtaagatcgtttctgaggaaattaactgctcagtgtactctgttgatccagaaggaagagtttacactcaggctatcgcacaatggcacgataggggtgaacaagaggttctcgagtacgagcttgaagatggatccgttattcgtgctacctctgaccatagattcttgactacagattatcagcttctcgctatcgaggaaatctttgctaggcaacttgatctccttactttggagaacatcaagcagacagaagaggctcttgacaaccacagacttccattccctttgctcgatgctggaaccatcaagtga

SEQ ID NO: 27 - DNA, 111 bp, Artificial sequence
  intein
atggttaaggtgattggaagacgttctcttggtgttcaaaggatcttcgatatcggattgccacaagaccacaactttcttctcgctaatggtgccatcgctgccaattgt

SEQ ID NO: 28 - DNA, 468 bp, Escherichia coli
cgttacaatctgcgcggtgagtttaacgtgccgttcggccgctacaaaaaaccctatttcccggaagcagagttgtatcacttcgctgaaaaagcgcagaatgcctttttctattgtgagtcttacgccgatagcatggcgcgcgcagatgatgcatccgtcgtctattgcgatccgccttatgcaccgctgtctgcgaccgccaactttacggcgtatcacacaaacagttttacgcttgaacaacaagcgcatctggcggagatcgccgaaggtctggttgagcgccatattccagtgctgatctccaatcacgatacgatgttaacgcgtgagtggtatcagcgcgcaaaattgcatgtcgtcaaagttcgacgcagtataagcagcaacggcggcacacgtaaaaaggtggacgaactgctggctttgtacaaaccaggagtcgtttcacccgcgaaaaaataa

SEQ ID NO: 29 - DNA, 597 bp, Corynebacterium diphtheriae
atggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctgcgatgagctaa

SEQ ID NO: 30 - DNA, 846 bp, Zea mays
tgctagtgaacctcaaggattgggggtgataaatgcgtgcttaatttttgaggatctagtaatcaagagtgagaggaggcaaaacatcgattcttcatagtgcttaaatagaaaagagtgataatactactcctttgttcgtcgagtactaaaagactactacatccattttacaattattttttagatacataaactttattattataaatctagacgtagttaagtgcaatgcaaacaacttatattttagtaatacataccattaataaataatactagtagatagtatatatatctaataagatgatattaaaggatgataataataacaattaataaatactactagtacacaaaagataagtttagcaacaattaagtttagtagtgcatgaagttgttttacgatattgataatatttatcacgcaaattttgtatattatagtgatgttttttgttccatatctatgttttatacaaattttttactgccgcaatgcactgcacatatctagttttagtactatatacaattaataaataatagataatactagcacatagtatatatctaatgaaacgatattaaaaggatggtaataatagcaattaataaatactagtagtatacaaaagataagtttagcaacaatcaaactaaaagatagccagtagaattttatttattttatattactgaaaacatcctcaagtgttcaccctgcagcccatcgcctattctatttaagaaatgcccgccctcccatactgctatcactcaagcctattctccattgtggaaccaacaaatctccaagctctcccaatttagaaacgagcc

SEQ ID NO: 31 - DNA, 1113 bp, Arabidopsis thaliana
atggtggaccaaggatttttcacactaaaaaaggaaaaaaagaaaaatatattaataaaacttttttatgttaaaatcttgggcttctgcttttgcgactcttggtcttcttcggacatggcacattccttaacctcactcgccgttttccagagcgtcatccgcaaagagatggtgaggagtttgcatgtctatgaatcggtggagattgagagagagttctggttcaagagcaaaagctgttatgtagagaagaaagcgaagcctctgtttcgttcggaagatttccggcgaccggagatctcggaagggtcggtttttggcacgtggcgttgtatctttgtgttccggtttaatcactcgcttcctcggtttcctactcttctctgtctttccagaaatcccaaactggaggacatccctaatttagccaacgagctcaagtttatctccgagttaaaaccatcaaagatttatgaagaagaacaatgcagtagcagtacagagggatattataactctgatctgcctaaaccacgaaagctcgttctgaaacaagatcttaactgccttcctgattcagaaaccgaatccgaggaatctgtaaacgaaaaaaccgaacattcggaatttgaaaacgataaaactgaacagtcggaatcagatgctaagactgagattttgaagaagaagaagaggacaccatcgagacatgttgctgaactatccttagaagagctttcaaaatactttgacctcactatcgtggaagcttctcggaatctcaaggtcggtctcactgttttgaaaaagaaatgcagagagtttgggattccacggtggcctcataggaagatcaaatctctcgactgtctcatccacgatcttcagagggaagcagagaagcagcaggaaaagaatgaagcagcagcaatggcggtagctaagaaacaggagaaactggagacagagaagagaaatatagtgaagagaccattcatggagatagggatagaaaccaaaaaattcagacaagaaaacttcaagaaaagacacagggcttctagagccaagaagaatcaagaatctcttgtcacttcctcttccacttaa

SEQ ID NO: 32 - protein, 370 aa, Arabidopsis thaliana
MVDQGFFTLKKEKKKNILIKLFYVKILGFCFCDSWSSSDMAHSLTSLAVFQSVIRKEMVRSLHVYESVEIEREFWFKSKSCYVEKKAKPLFRSEDFRRPEISEGSVFGTWRCIFVFRFNHSLPRFPTLLCLSRNPKLEDIPNLANELKFISELKPSKIYEEEQCSSSTEGYYNSDLPKPRKLVLKQDLNCLPDSETESEESVNEKTEHSEFENDKTEQSESDAKTEILKKKKRTPSRHVAELSLEELSKYFDLTIVEASRNLKVGLTVLKKKCREFGIPRWPHRKIKSLDCLIHDLQREAEKQQEKNEAAAMAVAKKQEKLETEKRNIVKRPFMEIGIETKKFRQENFKKRHRASRAKKNQESLVTSSST

SEQ ID NO: 33 - DNA, 2037 bp, Arabidopsis thaliana
atacaaaaatattttatagtagtgaactacgatatatatcattgtggactgacttgtggtgtgtgctgtctcagcgattagcaacctcacaaataaagttaatactaataagtaccctactgtttaacgacctcacaaatcaatactaataacttctaaatttgaaatttgttctctacgtttcacactacatttatggataatcgggtgtatctatagtatatgcatgcgttcgtatgagttttaataccagcgttgactgtcggcaagtaggaaataatccaattaataatacgtttgacaaaagattaaactgtagtactatatataatggaatatttaatccagatatcaaccgttgaaagttatctaatttaatttgataacgatttccaggactgtccccaaatctatctgaaagttattaatcactcctttctaaacaataattgaactttttcttaaaaaaacttctacgacaacacatttcctttgcataacgtagaagtcaatcaaagtttttaaatacttctatcaaatttttaagtaaaatagtattgacacgaaatgcaaaagacgaagtatactgaatataaaatatcacggctacaatgcaacatttaagaattagatgattggaaatcgatacagaaaaataatctaagagaattaggccgtcacttgtgttgtgtgggagcaaaacaaggaccaaaaatatcgggacaaataggttggtccaacctataggtagaggtagcccacttggcatagctcataataccattaccagctcatatgttttttcaaggattggagaaaattaaagaaagatgtaatcgattagagtaacagtggagtgctgaatttaagttagttaagaaaataattggtgttacttcttataaacttttaactcaaaaccaattcgtaatgaatagatagatccatgtctattatatcttatatactattcaaacctcttcttatatatttttccaatgtggattattcgcccatagataaaagataaaacttaacaattggtaagacaatatgacataaagtccttagttctacttacaaagaattttgtcaattaccttccaaaatttagatcttctaaaccctaagttattgggtttcaccaatataatgggtcatttcatctattcacccgaccgttagatttaccaatttctcatcatatctcgattttcaacatttaagaaagtaatcaagtttagccgaaatgcaagatgatacagaaacaatagcgtttaacggtgttagatgataaactcatcaactccattaagaaaaccaatcctgtaagaggtaaagaaggggagaccataattaatgtctaatactttcgtaatgaccactattaatgattagtactatgatctatgaagttgaagctctctttttttttttttttttttcccttcacgtccatagttagttacagcattgatgaaatttttgctgagaatagacgaccctttatcctccaccctacgctttaagtggttgggagttagaccctgccagatagattccaatcctaagataagtctgtttaacaaacctatcatatgtgaaagtgaaaaccattatgttgaagaattatctaaggcgtagagataatttctgcagcaaaaacatttttttaaacattgcgttatacattttaggatagtttatataatcagccaaagtgtatatttctgtaaaacacattactatcttgacatttttgtgataagctatataatcagtaacctgctacgtatagcttaaccccactattataattatgattcctcattcagtaaaactatatagctgaattaataaagtttattagggtctaatgaagttggtgtgatcatttaataatattgttatttcataactcggaattgaattatttattacccttgccatcttaaatctacatttgcaactcacccaaaagctttatcctttgtgttttttccactgtatactgaaaacaaatctgaggtgacgaag

SEQ ID NO: 34 - DNA, 1358 bp, Zea mays
acacaggaccaagaacttgaagatgcatttgaaggcctttatcttgttgactcccaagggccctagactttgtaatcttgcatttgtgctctgctgatctggtctgatactgatgtaactgatcaatgaactaattgtattagaactggattgtactctttttttcctttatatggttttctcataaggcgagtttttacctagaaaggtttttaataagacagccattgcacaaacagctataatattttatttaaagtctatgagactgactccgtgtgtgctactgcctactggctactactatctgtgaaattgtgacctgtgaactttgaaatgtgaaatttgtgacttgagaactatgattttatgacatatgaagttgtgaactgtgtatttgatacctgtgtgaatttatgacctatttaggccttgttcgtttacaccaatccagctctggattgacatggattggaattaaatacatgtcacaatctatgtcccaaaataatccaagcctactcatttttttatttggttaaacccatcatagattataacccaaggatttaggaaatttttaaactatggaagacatgaattctattcatagcttattaggtatggaataaatccatgaatatattgcacaagtttatattagaattcatgaatcaaaagaataactagttttgagagatacatggattaaatggtagatttaatctcactatgggattgagtgtgatatatggatttattcaatccaaatccggattaaatccatggtggatctatatatattggtgtgctcttagctcggttgtgtaggtgggccatgtttgacgtgccgagctggcacgatcggaccttttacccgtgccgtgctcgtgcaaggggtgttgcccgtcaggaggcaccgtgagttaatcggactcaattggaccggactcctcggatcgcgccgtgccgccgtttggatttctatacctgcacctgtggcctgtggggagtggggactgcgaatgacattcttgcatccctcctcaccaatcaaggcggcaacataccggccctttggccttccatgaacatgaacgcggcggaacgccacgccggcgtgcactactcacctgcatgaattcgccgcccactcacagcgccaacccaacttgaatgcacgcactaccatcaattcgccgccgcggccatcccttctgccagctgctatttatacgcctcgccccgctccagtctcagcagaaccaccagtcctccactccatcttctactccgaccacaaccacagcgaccacgaccgtgcacgtacgtacatgagcacaccaggcaacggcacc

SEQ ID NO: 35 - DNA, 30 bp, Zea mays
acacaggaccaagaacttgaagatgcattt

SEQ ID NO: 36 - DNA, 25 bp, Zea mays
gtgcctagcttattcgacgacctcg

SEQ ID NO: 37 - DNA, 25 bp, Zoanthus sp.
agtccaagcacggcctgaccaagga

SEQ ID NO: 38 - DNA, 25 bp, Zoanthus sp.
tacacggtgtcgaactggcagcgca

SEQ ID NO: 39 - DNA, 26 bp, Anemonia majano
atggccctgtccaacaagttcatcgg

SEQ ID NO: 40 - DNA, 29 bp, Anemonia majano
ggaggtgtggaactggcatctgtagttgc

SEQ ID NO: 41 - DNA, 1262 bp, Arabidopsis thaliana
gtttaggggtaatttagtttttaaaatatcatttatgtgttcttggaagtaacatattaatatcttaacatgaaaatctttggtcttggggttttggttttgcaaacttaattctctgatgttgaaatttgaccatctcttataatatttagaagtttgtgctttttgatagtccggaggagtatgaatgatcaatgaaccctttcaactgtgaaaatttcgagtagattaatattaataagagtaaaattttcattaaagaaaattttcactaaagaaacaaacaaaatatcaaattaactaaattaataaagccctcttttatcagaaaaggtggcctacttcaaatgttagggtgtcttattggtttgtgatttaaataaagtttttgtaacttaaagtgttatgtaaaatctgttgttattcaatcatttttatacaaagattttgatgtagtttagtgttatttgtttaagattttgtaaaaagtaatttaaaatcttcataaatctagaattattggattcatacttttataaaattaataaagttttgtgttgttaaattaaaacaaaaaatctataattgttaataaattaaattattatgttattagtttataactttctacactttattcataaaataaagttataaaaaatatcatcaaaataagagattgtttggaaaacttacaaaaatattaaaaaaaccaatcaacaaaattataaaaaataagtctctaataattatttaaaatctatttactttctataattttataaacgtcatcaaaattatcctcgtattagttttatctggtgactttgggcattttccctttctcataaaagggcgcgtgactcaaaattaatgtatagatgtcccataatttcattaagaatagattgttattttaaagtaacgtatcttttatttatgtagacaatattgttttcacgcatgtcttactaatgatgataatatataattaataatgaagacatttattaggtcttatcaattatcaggaaaaaaaagaaagacatttattaggtcaatttgctgacgctataaaagaaagaccttatcatttgattccaacacaattcatacaaacatcttccaagtaagtgatttggttttgatcaatctttaacaattttctcgtattacaacaccatcaaactaacaagtaacaacaatcattttttctattttatttgatgaaaagggaaatagtttggtgatttctcgtaaag

SEQ ID NO: 42 - DNA, 1834 bp, Arabidopsis thaliana
gctttaaagtcgtttatttttgtaacattactctctatttttgaaaaatgcgaaataatttttcaaagtaaaaaataatatgcaatttaggctttatacatatattataaacgttttttcgttcacatacatttgattttcaaaaatagaaaggtaagttgaacttttcgtctcgagttctttgaattgatatattacttatcaaattttaaaaaatatgagaaaacttaacaatagcaatattatgtattattttttactttataaaattattctgcaaatattgtgaattattttttacttcaaaaaattattttgtattcttttaagatgaaggataaagttataaaaatagacgactacaagaatttttttccacaaatctcctttttattcagatggtcaaacatggtcaaattgatacataatccacagaagttgtagagagattatagatgatggactctttgtatgtcattctgttttttcagacagctaaacgttatttaaaaaataaaaatacaatgcattaaaaacaaccatcctcgacttgtgctcacgcaacgctaccgtcttcatcattttaacctctctcgaccattttaacctctctcgaccctttttgtttttcattttttttaattaattattttcaaactaaccgaacccaatcaactaaatttacccctatttaactcaattttgaccagaaaaccaaaaagttcgattaatttcgataacaaaataaaataataacatggttcttaaacccaacccacacgaagaatcggactgccttttggggccacttggccattgtgtcaaccgggtttgaccacaagtcaattaaaaaaaaattatttaatatatttaatatttagaaaagttatatagtttatattaaataaaaataaaaatagtaataccaagtttaacaaaagtctaacaataataaacaactaaattttaattaaatttgatgaatactaaatcattgtaatattcgatcgtcattttagtctaacaataataatcaattaaaattttatttattatttttaagtccaactaaaatctaaaaccataacagaaatactagagatcattgatgacgaaaataaactaagaaaacatcacgaatttaaaataatgaattttgttttttctctctcacaattctattcattctttaaaagcgggattgtgaagtcttcaccaaatctaaaacattaaatgatgaaaaagttctaaaaataagtgaatatagtttgaaaccctagattctattccaaaatcaaatgaaaattttaaaacccatagccggcctgttttaatcgcttcaccagatcgcaagttaatgaagggtttttttgtggatttttctggttttagattgtcgagtattagttctaaacccaaataggaaaaatgtccgggtagcggattaccatgtcggaccggacggtccggatcaggcgtgaaaacaatgcatgtaatcgtattgtgtctaatatagtatttttgatttgtaataatttgaagaaaaaagagagtgttgttatctttaagtttgcccaaaatctacagtaatgttcgatcatagtctttaaagagagtgttgttatctttaaagttacaactttgtaaaattagcatagtctttaatataaacgtatcttaaacaaaattattaaatgttgaagttagtaacatataactattaattaatgaacaaatatcttttagtgattaacctataaaatctcttgttttcttgtttcatgtcatcaatcttacattcaatactaaaagtattcttacatccataaaaaaa

SEQ ID NO: 43 - DNA, 1248 bp, Arabidopsis thaliana
ttagtcagcaaaatcaaaatttaacatttaaataaagtctttatttaatattttatagcatttataatttgaaaatatgtaatgcaatgataaaaaataaaaataaaattctattatatactgaaatgatatccaactttttatacattccaaaactatatttggatgtctcttgatctcaactctgctcgtaggctatctaacaagtcagcagcaatataggtcttcagtgggccttattgggcctcattatgataagtaaagttctcgtagtggcctacaaaaattatattgaggggaccagataatagcttcacgtttagaagtttcataaagggaaaactcatatttcatttttgttattgttgacgtataaacaatccagatcatgaaaaaaaaaaagcgtataaacaatcttaaaattctaaccacttccaaattagtttttctcgaaactatttgtgcttttttgtttgttttgcttttgtggattttgattggagaagagaagaagaaatattatatgttttgcgtttgcatttaggttttttgtttgggtttagaaatattgaaactgatgtcttaactcttaaaatatatatttagcgctattgtctaacgttgatgtagtttggcatttactttttttaggtatgttgtatgcattagagttaattgtttgcttttgcattttcacatttaatttgaatgtgtttgcgttcaagataattaacattatttgtttgtgtgttttctttgaaattaagaagataatttgagctaccactgaattttgaaattagagaggcatcgagggaaacaaatcatatagtttggtgactgatttcaaggggaaataaccaaagaaggtcattagaagaataaatatggttagccagtattgattaggaagataatcaacatgttgaccacaatgaaagttagtcaatgaacggttttcaaataaagattacaaaataactagaccataaaaggtgatattctataaattctaattgttctttttatgtgttgtaataataattgttttattttaataactatatgtaaaaattattgtttatttatttcttatatattatggatgtcacgtgtataattatgaaaatccacgacttagaatgttcatgcattgcaattgtaagaaagcacttatgccttctatatatatattcgttgaaatgaaaacgataagagcacaaaaacaaaaacaaagtagaaaaggat

SEQ ID NO: 44 - DNA, 3674 bp, Sorghum bicolor
cggaccgaagctttcatgaatacggccttgctcctagggttgagcactatgctgcgcttgtcaatctcatagggcgacatggccagcttgaggatgcactggaggtgatcaagagcatgccaattgctccagaccgagctgtgtggggcgcattccttggagcctgcactgctaaaaagaatgaagtgctggctgcagtggctgccaatgcattatccaagattgatcctgagagttcagctccatatgttttgatgcataacttacatgcccatgaggggaggtggggaagtgcatctgtggttagagaagacatggaacggctagggattcacaagcatccagggtacagctggattgatctgcacgacaaggtgcatgtcttcatctcaggggatacctcgcatccccttacccaggagattttttcagtgctagaatgtttttataggtcatgtagagattggagctagacggccatgtgaaattgttatatttggagaagagaagaggttttgcggtgtagaaacaagctctttcttccgtttcttcttggcctatacatgtctcttgtaatgtttgtacctttctttggtaatgaaaacacaataattttattattacatttgataaaattgaagatccatctggttgggaaggctagggggatttgaaggactagttttcccaaacaataacccggcgacagtaggggtcatacgatgtcaattctaaccctctggtgcctatggatccaaagaaacggagtggtttttagagggcaggagaggtcaccattagacgtcctgagggacaacaaagacacagcatgctgctgggctttagctcgaccccagacggctgctccacctgcaattggttccctaggtagtgagtaatctcttttctgttttcatgccctagggcagcctagactgttttcaggggagcgctcctcgtgcgtgtatgctactattcagcttcctccttactattaatcaaagccggagttttccggatctttaaaaaaaagagagagataaaattgaagatctatgatggcactgctgattgtgtgaaaactaaagtactctcatacagatttccataatagtgatgtggctgtcaaatatttgcctgcaacttgaagaatttaaaatggttgaaattacatggagatgagccaactcaactgctcaagtaatctctcaccccctgccacttgaatggatacataattgccttttgcctatgcatgataattattgctgtaatgatcagttcataaatttatgactaaagtaaaaaccttagccttaacccaaatctatgatattagctcaggcaaagagtatatgctagaaatttctatcattttaattgagtagcactaatcctttgaaatgtgtaaaagaaaagttctagtatgatattagctcaggcaacccattgagtcacaactccgtgctacttctacttcccaatgaaaaaaatgccatgcatagatggcaaagactagcagtgctcctagattccttcgtgcaagtagaaacaaaatcttgaactgaatctagccggaaagactttgattgaccactatgcatgctctctaatgcacgaaccccaatggcatgctcggcaattaccaagagctaattatatctgtaactcccgatccattagccaccctttgcattaattcctcgcgtggtttttaatggccgtttccattaacccaatgatcccagggtttaaaagagccgcatttttccttccatcttgatcttctccatatattgctggcctcaactccgttccagcatctcctcccggaacccggaccgaagctttcatgaatacggccttgctcctagggttgagcactatgctgcgcttgtcaatctcatagggcgacatggccagcttgaggatgcactggaggtgatcaagagcatgccaattgctccagaccgagctgtgtggggcgcattccttggagcctgcactgctaaaaagaatgaagtgctggctgcagtggctgccaatgcattatccaagattgatcctgagagttcagctccatatgttttgatgcataacttacatgcccatgaggggaggtggggaagtgcatctgtggttagagaagacatggaacggctagggattcacaagcatccagggtacagctggattgatctgcacgacaaggtgcatgtcttcatctcaggggatacctcgcatccccttacccaggagattttttcagtgctagaatgtttttataggtcatgtagagattggagctagacggccatgtgaaattgttatatttggagaagagaagaggttttgcggtgtagaaacaagctctttcttccgtttcttcttggcctatacatgtctcttgtaatgtttgtacctttctttggtaatgaaaacacaataattttattattacatttgataaaattgaagatccatctggttgggaaggctagggggatttgaaggactagttttcccaaacaataacccggcgacagtaggggtcatacgatgtcaattctaaccctctggtgcctatggatccaaagaaacggagtggtttttagagggcaggagaggtcaccattagacgtcctgagggacaacaaagacacagcatgctgctgggctttagctcgaccccagacggctgctccacctgcaattggttccctaggtagtgagtaatctcttttctgttttcatgccctagggcagcctagactgttttcaggggagcgctcctcgtgcgtgtatgctactattcagcttcctccttactattaatcaaagccggagttttccggatctttaaaaaaaagagagagataaaattgaagatctatgatggcactgctgattgtgtgaaaactaaagtactctcatacagatttccataatagtgatgtggctgtcaaatatttgcctgcaacttgaagaatttaaaatggttgaaattacatggagatgagccaactcaactgctcaagtaatctctcaccccctgccacttgaatggatacataattgccttttgcctatgcatgataattattgctgtaatgatcagttcataaatttatgactaaagtaaaaaccttagccttaacccaaatctatgatattagctcaggcaaagagtatatgctagaaatttctatcattttaattgagtagcactaatcctttgaaatgtgtaaaagaaaagttctagtatgatattagctcaggcaacccattgagtcacaactccgtgctacttctacttcccaatgaaaaaaatgccatgcatagatggcaaagactagcagtgctcctagattccttcgtgcaagtagaaacaaaatcttgaactgaatctagccggaaagactttgattgaccactatgcatgctctctaatgcacgaaccccaatggcatgctcggcaattaccaagagctaattatatctgtaactcccgatccattagccaccctttgcattaattcctcgcgtggtttttaatggccgtttccattaacccaatgatcccagggtttaaaagagccgcatttttccttccatcttgatcttctccatatattgctggcctcaactccgttccagcatctcctcccggaacc

SEQ ID NO: 45 - DNA, 1808 bp, Oryza sativa
tttccatcctatcgagatgtactactccacttctgttctgtgcaggttgaatatatgtggcccaatcacatcttgccactaaaaatcttacatttatccatatactccacgaacagtagattttactcatccctgattagacccaaaacaatcatgagcacggtagacaacacaagcttagggcgtcttgcacgattaggttttgttcggtttagaggggattgaagaggattagaggggactgaggggtaataatttcacaccataataggtattgaataaatcccctctaatcccttcctcatgagaattaaccgaacaagcccttaccccgctacacccaaaaatgtttccgctggggtgcaatactgctatcgatggcttcttacgtaggaatttcatttttctaatattttttcattaaaaattgtacaaatatgacaaatctcttttataaaacaaaggtttctatagaaattatgcgagcacatatgttcacatatacacatatttcatatttatgactaattatttttttcaacgacaccgacaaatccgtcaataggctttatttttctttcacaaagcccgtaaacttccataggagcctactacatcagtggcttcgtgccgcactaacgaggcatctatagtgattgactttatcaatgtaaaatatgacagccaaatattttgatgggaggtgttcatggttatatgtacgtttatactccgtatgagtgagtagcactccctccgttctgagatatttactagtactacgaatctggaaatactctttattcagattcattgtactataaaagtatctcatatatccaaaaatttttatattttgagaccgagtgaatatatgtttgtggttttcctacatgtgagtagagtgcatcagtggatattagagcctccacgatatgggaatagtatcagccagtgtgttgatgacgtcaaagctcaaagggtagatgaaaagttcatgcttcaaaaatggcatgtcttggaaactgggattttcctaataatgagaaatcctatgtgcagagaggagacaaaagcactgctcaacacactgcaggctgcaaagatttgctagtactactactccagtacacaaacacatcattggccacttccctaatctcatttaacgtttgcataacgcactcattctgcggttactgcattagctactcatgaatgtggctatttactagtagtacaattctaagtgccattcccaggaggagtgagcagcttctccacccttaatcaggggcggagctaattggttttggcgatcaatctgcctcgtcgagtcgtcgttccgccctccacacttcccagttcgcgactgcgccaacgattgcgcgagcaccgctgccgcaactcaactcccgtgaccgacggcggcaatcggtggccggcgaggcagcgatcaggatcagggtaagtatatttcatctcctcctcctgtcctttggccctcccttctctgatccctcccgtcttcattaagctctaatcctaggtactaaattactaatttgattagtaagcggttaggccactagaacttgcgcccttgccgacggccaacacgacgctcgcaggccacaagacaaaagctgaatgaagcaccggcatcgcatgaactgatcgcattgtgttggtaaattctatacttctatgtcgacatattacatttatagtgttaaagaaaatttatgttcagttggaccatcctagcctaaaatcgtagctacgccactgcccttaagcccttgccc

SEQ ID NO: 46 - DNA, 582 bp, Arabidopsis thaliana
aaatctttggctttttggatcgttcttttgtggaaatggaatataaaacttttttgttacttcattaataacttatgattaattatgagaaatggaaattaaagatatatggccatgatctacaataatgttttaaccatacgtttcattttgttatcttaatcattcagttagtggttattaaacaatacataatcatgatcattgtgatgtgtatgtatgcgtatatataagaacatgtacattgagtagtactacactatttactcgaaatgattgcatgtcatatatgcatggagagacgaaaagaggagtctaatccaaatctaaacgcccctataaattacccactaattaacattaatcatatcttctcgtaactccaaatttaacacgacaatcaattagccgtcaatactcaataccccacttctcctaatagattcatcatcacttccattctttattctctctccatatcttactaccactagactctatcagtgatagagtatataaatcactctatcagtgatagagtttcacaacacaactactctatcagtgatagagtttacaatg

SEQ ID NO: 47 - DNA, 582 bp, Arabidopsis thaliana
aaatctttggctttttggatcgttcttttgtggaaatggaatataaaacttttttgttacttcattaataacttatgattaattatgagaaatggaaattaaagatatatggccatgatctacaataatgttttaaccatacgtttcattttgttatcttaatcattcagttagtggttattaaacaatacataatcatgatcattgtgatgtgtatgtatgcgtatatataagaacatgtacattgagtagtactacactatttactcgaaatgattgcatgtcatatatgcatggagagacgaaaagaggagtctaatccaaatctaaacgcccctataaattacccactaattaacattaatcatatcttctcgtaactccaaatttaacacgacaatcaattagccgtcaatactcaataccccacttctcctaatagattcatcatcacttccattctttattctctctccatatcttactaccactagactctatcagtgatagagtatataaactctatcagtgatagagtagtttcacaacactctatcagtgatagagtctttctttacaatg

SEQ ID NO: 48 - DNA, 582 bp, Arabidopsis thaliana
aaatctttggctttttggatcgttcttttgtggaaatggaatataaaacttttttgttacttcattaataacttatgattaattatgagaaatggaaattaaagatatatggccatgatctacaataatgttttaaccatacgtttcattttgttatcttaatcattcagttagtggttattaaacaatacataatcatgatcattgtgatgtgtatgtatgcgtatatataagaacatgtacattgagtagtactacactatttactcgaaatgattgcatgtcatatatgcatggagagacgaaaagaggagtctaatccaaatctaaacgcccctataaattacccactaattaacattaatcatatcttctcgtaactccaaatttaacacgacaatcaattagccgtcaatactcaataccccacttctcctaatagattcatcatcacttccattctttactctatcagtgatagagtctaccactagtctcttctctgaatgtagtatataaatcactctatcagtgatagagtttcacaacacaactactctatcagtgatagagtttacaatg

SEQ ID NO: 49 - DNA, 273 bp, Bacillus amyloliquefaciens
atgaaaaaagcagtcattaacggggaacaaatcagaagtatcagcgacctccaccagacattgaaaaaggagcttgcccttccggaatactacggtgaaaacctggacgctttatgggattgtctgaccggatgggtggagtacccgctcgttttggaatggaggcagtttgaacaaagcaagcagctgactgaaaatggcgccgagagtgtgcttcaggttttccgtgaagcgaaagcggaaggctgcgacatcaccatcatactttcttaa

SEQ ID NO: 50 - DNA, 1314 bp, Arabidopsis thaliana
ctgagaaggacatggtcggtgatcatacacggcgaggtggaaatgttatatttactattgaaaactaaattatttattatagagggagatattactctttacgctttcattaagatttatttttataagttttaaagtattttattgttatatgaagataaaatatattatttatttatattttattttataataagatattattttttatttttttttattattttatttttattctctgtgctatatatactctgaaagtctgaatatataatccattttggtgtgggagtattagactattaattatggtcaattaaatgaagttcaaaaatatgaatggaagatatatgaataaattgaattaatagatgtttataattattgagactgctttagcgtagaaaatgctgcatacattattgttgggaaaataaaaatgagtattaatatttaacataaatattaaatgtctttaatatgtgtgagagaattattaaaaaaaatcaacatttacgaaagagatggactataaacatttcgttaatacattttgttttttggtaaattggtttaatacaatatttttgaatcgtaaagtgttctggtaatatgatatgacatctaaatgaaatgattatgccagaagatcattgtcttgaatattggctgtattaacctctaacgaaattgagttaatatatattttgaatttaccatttgatatttagattgtataatttgagtttaccagctatatatcgtgttgaacttgcatgtaacacaccacttttttccaccgatttttgtttatggaaatataagtcaatatttattcgtcaaatacatatatactcacgcaaatatacgtccttaaagagaaaagagattttcatgattatttttgaaaaaagagaagattttgaaagatgacaacaagcaacgatatatgaacgcgcatagcatgtgatgggatggggcgggcctatgaaatttttgaacgtttacaaacttagggcctattattagaagatattactagcttttaataaacgaattatccctattaaccaaaataatcaacactaatcattaatttctacttactatctctctcgtaacttacagaaaacatataatgattttgacggctcatcatctcggagaactaaatacccacttcccacttatcatgtactttctctatctatgcatgtacgttaagttgtttatatatatatatacacacgattcattttccttgttttaagactaacgaacgttacaatctatctatatccactttcaatcgaa

SEQ ID NO: 51 - DNA, 654 bp, Arabidopsis thaliana
aacaccaatatgaagagaaaaaagcttgattctttctcattactcttcaagaactcaaaattacattgtgttttggtgtttcttcttcgagctcaaatcatcttggggttttcacagatttattcaaacaatgtactcccaagattattattgggagtattattatgtagtgcgaactcgatttgagaagtgaaaaaaagatggttacatttaaagcttttgatttgactacgttttctttgtttcatttactaagtaaattatcacttagtggagactctcattatctcttaatcatcttcaacatcaaatgtatctatcatcgtaacatataacacgtgcatcatctaatgcgataatacacaaaaactcaattcatttaatatcgattgtgaatttttagcaatatgatcttatcaactttcatgcattgactttgactagaggaagtagaaaaaaataatcgtcatcatcattaaagaagcaactaacctacacacaattcagcccccgtgatcatatatacttaattaaagtcacacggtaattaattaagattaacatttaatgatttctaatacgctttgggactcgtaactcccattacattgcaatccctatgaacattcatctttgtttttacagagactatat

SEQ ID NO: 52 - DNA, 2164 bp, Arabidopsis thaliana
aatcctcttctttagggtttctttccgactttgaatacactctctgcttttttttctgctttctaaaaagtcttcaacactttgctttctctcatcttcttttttttttccctctttttttttaacctttctttagaccacgtgagaaagataacttccactttaaacacttgtcctcttctgtcttattgtcttgtcttgtcttttcttgataggcttccattattgtggctagggcccaaaaaggccttaaagcccaaagcttcgtggtttttcttctcttgtggtttaggctttacagtgatcagagaaacccaaaacacgttggaaacgtctaagcagagaaaaacagagcttccaacaaattcagcattgtaattcttctagacgttttatacaaattttacatatacactatggaactctccttgcatttctaccaaatctgaattgaaaaagggatttgtaagatatgaaaatgcgataacgttgcctagattaatcagttttcgacattttttttttcctgttccgattccatgtaactttttgagggccacaacttttcttaattaaaaaaataagaaaaataaaagctcaagtgacaatcagtttttgaaaatgatactaactaagctcttaacatttttacgcatgtatataaacattaatcttttatttggtcttaaatacaaagcatatatatgatgctatcaatctaaatggtctatttgtacataattaaataaaacataaaattaaagcctgcgcatacaacatgtctacaaccaaaaacttctttcgtttatatcaaaatcaacatcccaatacttcatcttctcttctcttctatttggcacttatagacgcgaaaggtttgaaccggcgggaaagtaagacaccataatcggagctctcggggatttgctttttggtttctttgaggacaggactttcaagtcactctcatcagttgagctaatctttgagtctgattttggaacaaagcaatcaaagacggaggcaaagagagaacacatgatcataggagtttgaaaaacgtgtttggagtctatatacgatgaatgatatgattaagatttgatctcaggtaattacgtgaacgatatgtatttataagacaacccatctttataaattcttggacacgtttctaggaaatgaccactaaatcttgctggccaagctttgccctattcttaattgttttctcttttgacaacacttgggcaccttttttgactctttgggcctaattggaccaactattgataccaaacatacgttaacatcacctccatatcactcacccaatcaagttttccaaaatgttatgattaaaattagggtcttcatgtcactatccaacaaaagttttccaaaattcaacattaaaattaggggaaatatgtacgaaatagaacttatatatccatgttaagaagaaaaaaaactatatatccaagcaatacaaaatatttaggttctacactccattttatacaaaatattaattgttttcgattagagttttattagaaagttctcactcagataaaatcaaaactagtactctgtatttttatatagagaaaaatccttgtaagttaatgttactaatactacccaagtacccagagtattttgacacattctattgacttttgattgaaacatgtccggcttaatttaacgcaattattcagtttagattttgaacaccttaaatgaattggcttttaacagatcataatatcaataccagttttagtccttgagaactcagcccatgacttaaaatatgaaaacttcagcccatgacttaataaatgaacaaagagaacccaaaaacagaaaatgaatcatggacatatttacatatatcataatctgaccaaattggaaattatgctcaaatgcttaatattcctctgattcatttaccaaattcaacctctgtagaatcattctaaacaaaattcaattaccacttttcagacatgcgtcgcgcgtgtgagtgtttagctacatgggcttggttcggtgcaacccgcttcccactgttaattttacataactaccctcgcacgctccgcttgcctacacgtgcgttccggaatattctgcctttttggtaatttcg

SEQ ID NO: 53 - DNA, 978 bp, Arabidopsis thaliana
gtacagggaaaaatgcggtgtaaataccaaactttacgaagcgtggcaaaaatgttataaaaaaaaaatctataaaactttgttattgtgatgtgaaggaatcgccctagtcaacaaattaaatcacaatcacctcatgaacacaactgatttaactatatcaactttttcttgaaccaaaggtaccaagtacaaactaataacgatacgagttgtcagttgtgtaccaagtttttactggcaaataaatcgacttgctaccaaagtaccaactaatacgagtgtctctgttttgttaacttgacccaatctttcttcctcgtctctttgcaaaacgcttaagcccaaatataactaatatggcccaaaatattcttgagagatccaaacctataactcgaatacccggtaggacaaaacgcttcatgtcatattctgacactttttaacacttcatgatcggtatttaaatagcattttcatttcttgtataacaactgagttcatatatatacatcattgatcatatattgagtattgatctaactaattcataatcaactattcaactgttttcattaaaaaaaacaagtttcgtatataaaacttggaaatattgtttttaattaatttgaacgtacattgttatgggttcttctaatgttaagaaaacaccaaagagagaaaaaagggtggtcaaaaaacaaatttagaaatcaatgctataattaagctatgataaactaatcatttttttatcgaaacgtaatgaaactaattttaaattttaacaatcaacgattttacttttttgtctcagtctaaaaataacaatcgggtttctaatataaaacaaactcggtgctccacgagaatagttgtcctcttctcaaacatatctcaacttattgtttgaatataaaaagagatatcaaaaagaagagaagaccaaaaacaaaacaaaaatctctaataacc

SEQ ID NO: 54 - DNA, 524 bp, Arabidopsis thaliana
gaaaattgtgcaaaagctttcatgtgcggctcagattaattagtcatttactactaataaaactttcactttggggtctagtagataattctccacccccattgaatctttttagtggaggtctaaacatacataagattctatagattgacatttgggaaaccatcctcatacaaaaaagacctaaacggaatctatgaagaattattaacagaaaagaaaaacagatggcaatgagaaaagatcgggttcaaggaaaacacagccgtacaaaactcaagaacaaaaacaccaaaaataaacaaaaaaacttccaaaaatagatatagaatcacatggttttgttgttttgtctatttgttctctataaaaggagatatttggttggatctatcatagcgtctcctctcaaccaaagcttacaatttgttctcccttaaaaactaaattttacaaataaactctcaaatccaagagaggagaagaccgaagtaaaaacacaagaaaaaaaaggattaaggcac

SEQ ID NO: 55 - DNA, 995 bp, Arabidopsis thaliana
atttagaagtgggaatgggtctatgaaatgagattacgtcaatatgagtgaaattgataaattatccaatcccataaacgagatggtgaacaaatataaatttacatttactgctagtaaatacaactacaattactttttaccacgcaaaaggagagaggagagattttttttttttttttacttcgtaaggataatatgtacttagaaaataatatacagtgacgaaggatgatgaatgctttcatgggaaacgagcaattgaccaggttgagagagatatgggccgattaaagctgtcactgtctctgttatgacagaactaagttcacgtttacgtgatttaaatttttattgatagaggagatgattgtgtttacaatcactgaattgttactgattttactgtgaattgcatatcaattggtaaacctgtaaaattgtcttatcattttgtggattaccaatcatatttatgagaaatctcaattccatttacataaatatttaaaagacaattacagaataatttagctatgacgctccgacataatcaacaaacaaaacaatattttgcatctgtatatatatatatacaaaattttgttacacatacacataattttgaggaagaaacaaaaattattatttggttgcaattttagactgttttataattaaccgagtaatattgatcattctcaaccacttaatcaattgattctttttttttttttttttgcttgatataaaaaaagttacggtaaaattggaaatcgttactacctaagattggggtcaacaatccgtaaaagaagatggaatcacacactgtaataccaatacttttctataaggaatcaaatctataaatagcatactaactagcactataaaaacattatgaatcctcctatgagcaaatcacttttaaatttgttaacactcttttaaaagaacaaaaaaagcaaaaaaaaaataaagatattatcacc

SEQ ID NO: 56 - DNA, 1783 bp, Arabidopsis thaliana
actcaaaaggcatagctacattaattctcagaaaatcatcaaacaaatactttatgttataatcactagctagtaaatgtttttttttttttgtaaaataaaatcaagattggtatagggcaaccacagatctattgatcgacctatgctaggataactctgtaaaaacaaatatagattgtaacaaacattcagaagtgaggcgagctcacattaataaaagtttttgataattttcgtctcaacacaaaagtaattaagcagttataatcttttaccatatttcataattatgatcgctacattaaaaaaaaaatctacttcaatttcatttttcatttttatctttgcaatgacctaacacaaattcttccatgagatcaaccttttcataagaaagggagattgaatcaaagaccaccataataaattaaaaatactgtccaagaaaaaaatagttttgttgacgccaatgatcgaatatgttataggattgtgcttttttctatttttgcgggtaattgtgaggttacttcatgaaagaagatcaacaatctttgcggccaatttggtaagctacaaaactaagcctatgtctgagcagttcacgtaagcttctctagtggctcttcaatccaattttcaaactaaacgtgtgatttccacacttaaatctcacgtatatttattcggttcttatggttccgagacaggttctggtctagtgtaactgagaaaagctccttataaatttctgcatgtttctatttttaaccgtttgcatgcaattcatacaagtttagtaagggtttttttttggggtcaaagatgccagttttagtagttcttaaaccgattttgtaaaagctatggacgattcgaatttatctcctcggaagattgtatataaaccataatttatacgaatgattgatttttggtagtttaattggtctttgtgagtgttcttagacttttctcttgatggttgtttgatcttaaaacatttcccatgtgaagtctaactctcttatagtattatacaatagcaaaaacatgttagagattttaagagaattgaatagtttaattattttagtcaacttattttagtttaaaccttttaacatttccaccatcatacaaataaactatttaattaacactttgtaaggtgtaacactttttagcatgtatgcattatatattattttgtttaactcagtgaagtattcatctgaatacaagttaactatgaatatatagtcctgtcttcttacatgaaagagtcatattttaataccacatagcaacagcaataatattgttacatgctataatatcagagcatccacaaagacaattggtccactagtcagagatgtacctagcttatgttgagcgacaagaaatcaaatattttggtacgtacagtgatcaacatgtgaatagtaagatatgcaacccgatatacagtcatttacataactagattgatgatccataaagaccgaaaaagtagtggtcataaacgaatgttgcacaaattttgtttaagagtcagttacataataatttgcatctaaatatagattaaagaaaaatgcggatcacagcaatagaaattgccgtcaaaatagagagtgaaacaagagaacctcttttgctattcaattgcaaccttaaaccaatccaccattttctcttattcacataaaaaatagagttttaaccatctatataaaccccacctcacctagaaagtaaaatcatcccaaaagga

(19) United States
(12) Patent Application Publication
(10) Pub. No.: US 2013/0180005 A1
(43) Pub. Date: Jul. 11, 2013

(54) Method to Screen Plants for Genetic Elements Inducing Parthenogenesis in Plants

(71) Applicants: Andrew Mark Cigan, Johnston, IA (US); Shai J. Lawit, Urbandale, IA (US)

(72) Inventors: Andrew Mark Cigan, Johnston, IA (US); Shai J. Lawit, Urbandale, IA (US)

(73) Assignee: Pioneer Hi Bred International Inc, Johnston, IA (US)

(21) Appl. No.: 13/445,276

(22) Filed: Apr. 12, 2012

Related U.S. Application Data
(60) Provisional application No. 61/583,641, filed on Jan. 6, 2012.

(51) Int. Cl. A01H 1/06; A01H 5/10; A01H 5/00

(52) U.S. Cl. 800/278; 800/298; 800/306; 800/322; 800/314; 800/317.3; 800/312; 800/320.1; 800/320.3; 800/320.2; 800/320

(57) ABSTRACT

Compositions and methods for producing a plant population lacking sexually derived embryos are provided. Compositions include suppression cassettes encoding polynucleotides and promoters resulting in parthenogenesis. Further provided are parthenogenesis genetic elements used to prevent sexual reproduction in self-reproducing plants.

Methods include: utilizing maternal embryo defective recessive mutations which are maintained as a sterile inbred maintenance system, allowing generation of populations that are homozygous for recessive mutant alleles, but transgenically complemented. Methods include utilizing a toxin genes expressed via egg-cell specific promoters, creating a dominant, embryo-less phenotypes, non-transmittable through female gametes. Resultant hemizygous plants are transformed with egg-cell promoters driving the antidote, a pollen ablation PTU and a seed color marker for identification of transgenic seed. The generation of a plants 50% female fertile, having seed which when grown in the next generation will yield plants with 50% viable transgenic seed, and 50% non-viable embryo-less seed.

### CROSS-REFERENCE

This utility application claims the benefit U.S. Provisional Application No. 61/583,641, filed Jan. 6, 2012, which is incorporated herein by reference.

### FIELD OF THE DISCLOSURE

The present disclosure relates to the field of plant molecular biology, more particularly to plant female reproductive biology, methods of altering plant female reproductive biology and screening for altered mechanistic capacities for reproduction.

### BACKGROUND OF THE DISCLOSURE

Apomixis refers to asexual reproduction leading to the production of seeds without fertilization, leading to offspring genetically identical to the mother plant (Koltunow, et al., (1995) Plant Physiol. 108:1345-1352; Ravi, et al., (2008) Nature 451:1121-4). It is a reproductive process that bypasses female meiosis and syngamy to produce embryos identical to the maternal parent. Apomixis increases the opportunity for developing superior gene combinations and facilitates the rapid incorporation of desirable traits. Apomixis not only provides reproductive assurance, but also avoids a loss of heterozygosity in the offspring because the off-spring maintains the parental genotype. Apomixis therefore avoids the effects of loss of vigor due to inbreeding and may additionally confer some advantages because of the heterosis affects.

At the species level, apomixis occurs in less than 1% of the species. Apomixis occurs in many wild species and in a few agronomically important species such as citrus and mango, but not in any of the major cereal crops (Eckhardt, (2003) The Plant Cell 15:1449-01). One form of apomixis is adventitious embryony, where embryos are formed directly out of somatic tissues within the ovules outside an embryo sac. Adventitious embryony usually occurs in parallel to normal sexual reproduction. A second form of apomixis is diplospory, which displaces sexual reproduction. In diplospory, an unreduced egg cell is formed which then goes through a process call parthenogenesis (embryogenesis without fertilization) to form an embryo. A third form of apomixis is apopsory, which like adventitious embryony takes place in tissues outside the sexual embryo sac. Apospory, involves the formation of an asexual, unreduced embryo sac which like diplospory goes through parthenogenesis to form the apomictic embryo. All three forms of apomixis rely on the production of an embryo without fertilization (parthenogenesis). Because it offers the promise of the fixation and indefinite propagation of a desired genotype, there is a great deal of interest in engineering this ability to produce clonal seeds into crops, especially cereals (Spillane, et al., (2001) Nat. Biotechnol. 22:687-91).

A molecular approach to engineer apomixis in commercial plant lines is highly desirable. Regulation of gene transcription plays a substantial role in expression of seed-specific developmental programs. Therefore, the regulation of the molecular switch during early ovule development, at the point of divergence between sexual reproductive pathways and apomictic processes, is a point at which apomictic-like traits can be controlled.

The disclosure describes a way to maintain a large plant/seed population that lacks sexually derived embryos. This can be useful for creating a screening population for genetic elements that induce parthenogenesis. Additionally, once the parthenogenesis genetic elements are identified, this same approach could be used to prevent sexual reproduction in a self-reproducing plant.

### BRIEF SUMMARY OF THE DISCLOSURE

There are two distinct, but similar approaches. The first approach utilizes a maternal embryo defective (embryo lethal) recessive mutation which is then maintained in an approach similar to that used in the Sterile Inbred Maintenance System (SIMS) aka Seed Production Technology (see, U.S. Pat. Nos. 7,696,405, 7,915,398 and 7,790,951). This system includes introduction of a transgenic cassette which has three parts: 1) a wild type allele to complement the embryo lethal mutation; 2) a pollen ablation plant transcriptional unit (PTU) to prevent transgene transmission through the pollen and 3) a seed color marker PTU to allow removal of the transgenic population from the seeds produced. This will allow the generation of a population that is homozygous for the recessive mutant allele, but is transgenically complemented. These plants would segregate, based on these changes, 1:1 in the next generation for viable transgenic seed and non-transgenic, non-viable, embryo-less homozygous mutant seed.

The second approach can be accomplished in a similar manner using a toxin gene and an antidote gene. In this system, the toxin gene would be expressed via an egg-cell specific promoter (Construct A) creating a dominant, embryo-less phenotype that cannot be transmitted through the female gamete. Construct A would be transformed into plants previously transformed with a transgenic cassette which has three parts (Construct B): 1) an egg-cell expressing promoter driving the cognate antidote; 2) a pollen ablation PTU to prevent transgene transmission through the pollen and 3) a seed color marker to allow removal of the maintainer population from the seeds produced. This will facilitate the generation of a population that is homozygous for the Construct A, but is 50% female fertile because of the hemizygous Construct B. These plants should segregate 1:1 in the next generation for viable transgenic (AA/B−) seed and non-viable, embryo-less AA/−− seed.

These systems are designed to be used in screens for genetic elements which induce parthenogenesis in seeds. In addition, the systems could be utilized to facilitate production of self-reproducing hybrid (plants that do not lose hybrid vigor) with the addition of the parthenogenesis PTUs and removal of the complementation or antidote constructs. A non-exhaustive listing of components might include: a recessive embryo-lethal mutant/egg-cell ablation line, a wild-type complementing transgene/egg-cell antidote line, a pollen ablation transgene, a seed color marker, and (for self-reproducing plants) a parthenogenesis PTU.

Previous solutions to screen for somatic embryogenesis include using male sterile lines to identify fertilization independent seed formation (not successful in identifying parthenogenesis genes) and screening activation tagged somatic tissues (roots) for embryogenesis (Zuo, et al., (2002) Plant J 30:349-359; Wang, et al., (2009) Cell Res 19:224-235). The referenced methods, however, do not identify genes which produce somatic embryogenesis in seeds. There have been no successful similar methods for maintaining self-reproducing plant crops. These approaches describe systems of producing large populations of seed without embryos which could be screened for parthenogenesis in the context of a seed. One advantage of these approaches is that fertilization of the endosperm will not be prevented (unlike the male sterile screens). This disclosure provides a superior approach because the nutritive endosperm is required for normal seed/embryo development.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a fluorescent image of a fertilized Arabidopsis embryo sac with only remnants of the egg/zygote (red) and of the synergids (green). Breakdown remnants of green and red may appear yellow. Central cell appears healthy with 3-4 endosperm nuclei indicating that fertilization did occur

FIGS. 2 through 8 depict several events from the same transformation construct.

FIG. 2 is a fluorescent image of a fertilized Arabidopsis embryo sac with a zygote (red) that is in the process of breaking down, losing integrity and appears to be “blebbing”. The persistent synergid (green) appears to be condensing and breaking down as well. Central cell appears healthy with several endosperm nuclei indicating that fertilization did occur.

FIG. 3 is a fluorescent image of a fertilized Arabidopsis embryo sac showing 7-8 endosperm nuclei in a normal developing central cell. No sign of a zygote or embryo (red) nor any sign of a synergid (green) is present. The endosperm may be described as developing in the absence of an embryo.

FIG. 4 is a fluorescent image of a fertilized Arabidopsis embryo sac with a remnant of the zygote (red) and the persistent synergid (green), where both appear to be condensing and breaking down. Central cell appears to be unhealthy and in the early stages of breaking down as is indicated by the increased vacuolation of the central cell.

FIG. 5 is a fluorescent image of 2 unfertilized Arabidopsis embryo sacs just prior to fertilization. The embryo sac at left has a central cell (cyan) with the 2 endosperm nuclei and 2 synergids (yellow), but is lacking an egg (red). The embryo sac at right has a central cell (cyan) with the single primary endosperm nucleus, but is lacking the synergids (yellow) and the egg (red).

FIG. 6 is a fluorescent and differential interference contrast (DIC) fluorescent overlay image of a fertilized Arabidopsis embryo sac. The central cell (cyan) has the single endosperm nucleus and 1 synergid (yellow), but is lacking an egg (arrow).

FIG. 7 is a fluorescent image of a fertilized Arabidopsis embryo sac with 4 endosperm nuclei in a normal developing central cell. Only a very weak red fluorescent signal (arrow) indicative of a remnant of the embryo or zygote is present. The persistent synergid (green) is breaking down. The endosperm is developing in the absence of an embryo.

FIG. 8 is a fluorescent image of 2 Arabidopsis embryo sacs with well developed endosperm. The embryo sac at left has numerous endosperm nuclei in its central cell (cyan) and at its micropylar end (arrow) is a remnant of the embryo or zygote (red). Under normal conditions this embryo should be much more fully developed, at the heart-shaped stage. The smaller embryo sac at right has numerous endosperm nuclei (cyan) but is lacking an embryo (arrow). Synergids are naturally degraded by this late stage.

FIGS. 9 through 11 depict seed from a maintained embryoless population of seed.

FIG. 9 is a widefield micrograph of Arabidopsis seeds segregating for the embryoless condition. In this sampling brighter seeds are plump and contain embryos. Darker seeds are shriveled and lacking embryos. The embryoless seeds have develop significantly, consistent with the substantial endosperm development in the absence of an embryo.

FIG. 10 is a widefield fluorescent micrograph of the same sample field as in Figure A. Bright red fluorescence is observed from the plump, embryo-containing seeds. Little or no fluorescence is observed from the shrunken, embryoless seeds.

FIG. 11 depicts the components of one envisioned embodiment of a library construct designed to screen for parthenogenesis. Within a TDNA backbone, a promoter such as AT DD1 PRO (an antipodal promoter) would drive a cDNA or gDNA fragment from an apomictic genetic source bordered at the 3′ by a terminator. Also within the TDNA borders is a seed color marker unique from that of the maintainer construct.

FIGS. 12 and 13 depict a method of screening a parthenogenic cDNA population and the prophetic identification of a parthenogenic embryo among the screening population.

FIG. 12 depicts a green-fluorescent, parthenogenic embryo developing from antipodal cells. A prophetic green fluorescent seed without red fluorescence is produced and identified in a high-throughput screening system such as the Complex Object Parameter Analyzer and Sorter (COPAS) from Union Biometrica.

FIG. 13 depicts ˜15,000 embryoless/maintainer population seeds analyzed on a COPAS. The data skews toward red fluorescence on a logarithmic scale. The green polygon and a single data point prophetically demonstrate the identification of a green-fluorescent parthenogenic embryo containing seed within a defined selection criteria.

FIG. 14 diagrams PHP57122, the vector used for the super-transformation of PHP47029/PHP50940 (embryoless line) plants. Prior to Agrobacterium transformation, the ATTR1//CAM/CCDB/ATTR2 is substituted with a cDNA from a heterologous source. The resulting TDNA drives the cDNA expression in antipodals from the AT-DD1 PRO and drives AC-GFP1 expression in embryos from the KTI3 PRO.

FIG. 15 shows PHP47029/PHP50940 (embryoless line) mature seed sorted on a Union Biometrica Complex Object Parameter Analyzer and Sorter (COPAS) after transformation with a cDNA expression library intended for screening for antipodal parthenogenesis. X-axis=green fluorescence; Y-axis=Red fluorescence; Blue=single data point, Red=two data points, Green=more than two data points. A data point tail can be seen skewed toward the right which represents seed with red and green fluorescence due to transformation with the cDNA expression library. The polygon is the zone selected for sorting hits; six putative hits were selected in this screen shot during screening on the COPAS.

FIG. 16 depicts PCR results from six putative hits of PHP47029/PHP50940 (embryoless line) mature seed sorted on a Union Biometrica Complex Object Parameter Analyzer and Sorter (COPAS) after transformation with a cDNA expression library intended for screening for antipodal parthenogenesis. Nested PCR was performed on crude seed isolate using primers flanking the cDNA insertion site in the antipoadal parthenogenesis screening vector, PHP57122. The PCR products were run on a 1% agarose gel in TAE with Ethidium Bromide staining. A common 1.7-1.9 kb band was observed from 3 of 7 putative hits.

### DETAILED DESCRIPTION

The present disclosure now will be described more fully hereinafter with reference to the accompanying drawings, in which some, but not all embodiments of the disclosure are shown. Indeed, these disclosures may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Methods and compositions are provided which prevent formation of a sexual embryo in a plant ovule and to use this state to identify and promote asexual embryo formation (parthenogenesis).

Various methods can be used to assay for such a transition to an embryo cell-like state. For example, egg cell-preferred promoters can be operably linked to a marker. Such a reporter construct would be inactive in most ovule plant cells, but the reporter construct would become active upon the formation of the embryo cell-like state. Embryo cell-preferred promoters which can be used to detect an embryo cell-like transcriptional state include, for example, the Arabidopsis thaliana down regulated in dif1 (determinant infertile1) 45 promoter (AT-DD45 PRO; SEQ ID NO: 10) and the EASE promoter (egg apparatus preferred enhancer promoter; SEQ ID NO: 19), the KTI3 promoter (Perez-Grau and Goldberg, (1989) Plant Cell 1:1095-1109). See also, Yang, et al., (2005) Plant Physiology 139:1421-1432. See also, U.S. Provisional Patent Application Ser. No. ______, entitled Ovule Specific Promoters and Methods of Their Use, filed concurrently herewith and herein incorporated by reference in its entirety. When employing such embryo cell-preferred promoters operably linked to an appropriate marker one can assay for an embryo-like transcriptional state by assaying for expression of the marker in ovule cells. In this manner, an embryo cell-like state can be assayed for in tissues of the plant ovule including any tissues and substructures suitable for parthenogenesis.

Additional female gametophyte-specific marker genes that can be monitored to assay for an egg/embryo cell-like state include any female gametophyte-preferred expressed genes, such as, AT1G18770 (MYB98), AT1G26795 (Self incompatibility protein-related), AT2G20070, at4g25530 (homeobox protein, fwa) and at5g40260 (nodulin mtN3 family protein). See, for example, Koszegi, et al., (2011) Plant Journal 67:280-291, herein incorporated by reference.

In another embodiment, an egg cell-like state may be indicated through the development of a cellular morphological state like that of a zygote, notably the polar distribution of dense cytoplasm occupying much of the cell volume with a nucleus located at the densely cytoplasmic apical end of the cell opposite a large vacuole which occupies a medial to basal position within the cell. One embodiment would include an Arabidopsis cell similar to the natural zygote cell size of approximately 26 μm tall×15 μm wide. Such morphological embodiments are supplementary to molecular determinants and would not be diagnostic of an embryo cell-like state independent from other determinants.

In still other embodiments, an embryo cell-like state can be characterized and assayed for the development of embryo-like structures in tissues and substructures outside of the embryo sac, including the formation of such structures in any tissues and substructures suitable for parthenogenesis. An embryo-like state can be characterized by a contiguous grouping of cells displaying the morphological developmental states of an embryo. Morphological characteristics of a embryo-like state may include typically vacuolated cells becoming densely cytoplasmic, or isodiametric cells becoming elongate and egg or zygote-shaped. Other cytological features suggestive of an embryo/zygote-like state may include changes in polarity of the cell, the “apex” becoming broad while the “base” becomes attenuated and tapered. Other features may include the majority of the cell's cytoplasm occupying an apical position while a large vacuole occupies a medial to basal position within the cell. The nucleus of this zygote-like cell would occupy an apical position within the cell. In the example of Arabidopsis, the morphological states would be an egg, zygote, proembryo, globular or heart-shaped embryo, torpedo, walking stick and curled cotyledon. Development of a suspensor or cotyledon(s) would be another morphological embodiment. Such structures may also express molecular markers such as the expression of AT-DD45 up to the early globular stage. Later globular stage through maturity, the embryo-like structures may express a KTI3 reporter or other embryo specific marker expression.

In specific embodiments, the “egg/zygote/embryo cell-like state” can progress into the creation of parthenogenesis or initiation of embryony. Such methods and compositions are discussed in further detail elsewhere herein. In adventitious embryony (sporophytic apomixis), an embryo is formed directly out of the somatic tissue within the ovule that is outside of the embryo sac. In other words, the embryo is not from a gametophyte, but rather is formed, for example, from the nucellus and/or integument tissue. In incomplete embryony embryo development is incomplete. In some embodiments this may indicate a lack of a suspensor. In other embodiments this may indicate an arrest in embryo development prior to maturation. In yet other embodiments, this may indicate a lack of a globular head, cotyledon or other embryo organ.

TABLE 1

| | | | POLYNUCLEOTIDE/ |
|---|---|---|---|
| | | | POLYPEPTIDE |
| SEQ ID. | NAME | DESCRIPTION | (PN/PP) |
| SEQ ID NO: 1 | AT-NUC1 PRO | OVULE TISSUE- | PN |
| | (AT4G21620) | PREFERRED | |
| | | PROMOTER | |
| SEQ ID NO: 2 | ALT-AT-NUC1 | OVULE TISSUE- | PN |
| | PRO | PREFERRED | |
| | (AT4G21620) | PROMOTER | |
| SEQ ID NO: 3 | AT-CYP86C1 | OVULE TISSUE- | PN |
| | (AT1G24540) | PREFERRED | |
| | | PROMOTER | |
| SEQ ID NO: 4 | ALT-AT- | OVULE TISSUE- | PN |
| | CYP86C1 | PREFERRED | |
| | | PROMOTER | |
| SEQ ID NO: 5 | AT-PPM1 PRO | OVULE TISSUE- | PN |
| | AT5G49180 | PREFERRED | |
| | | PROMOTER | |
| SEQ ID NO: 6 | AT-EXT PRO | OVULE TISSUE- | PN |
| | AT3G48580 | PREFERRED | |
| | | PROMOTER | |
| SEQ ID NO: 7 | AT-GILT1 PRO | OVULE TISSUE- | PN |
| | AT4G12890 | PREFERRED | |
| | | PROMOTER | |
| SEQ ID NO: 8 | AT-TT2 PRO | OVULE TISSUE- | PN |
| | AT5G35550 | PREFERRED | |
| | | PROMOTER | |
| SEQ ID NO: 9 | AT-SVL3 PRO | OVULE TISSUE- | PN |
| | | PREFERRED | |
| | | PROMOTER | |
| SEQ ID NO: 10 | AT-DD45 PRO | EGG CELL-PREFERRED | PN |
| | | PROMOTER | |
| SEQ ID NO: 11 | ATRKD1 | CDNA OF RKD | PN |
| | FULL LENGTH | POLYPEPTIDE | |
| | CDNA | | |
| SEQ ID NO: 12 | ATRKD1 | RKD POLYPEPTIDE | PP |
| | AMINO ACID | | |
| | NM_101737.1 | | |
| SEQ ID NO: 13 | ATRKD2 | CDNA OF RKD | PN |
| | (AT1G74480) | POLYPEPTIDE | |
| | FULL LENGTH | | |
| | CDNA | | |
| | NM_106108 | | |
| SEQ ID NO: 14 | ATRKD2 | RKD POLYPEPTIDE | PP |
| | (AT1G74480) | | |
| | AMINO ACID | | |
| SEQ ID NO: 15 | ATRKD3 | CDNA OF RKD | PN |
| | (AT5G66990) | POLYPEPTIDE | |
| | FULL LENGTH | | |
| | CDNA | | |
| | NM_126099 | | |
| SEQ ID NO: 16 | ATRKD3 | RKD POLYPEPTIDE | PP |
| | (AT5G66990) | | |
| | AMINO ACID | | |
| | NP_201500.1 | | |
| SEQ ID NO: 17 | ATRKD4 | CDNA OF RKD | PN |
| | (AT5G53040) | POLYPEPTIDE | |
| | FULL LENGTH | | |
| | CDNA | | |
| SEQ ID NO: 18 | ATRKD4 | RKD POLYPEPTIDE | PP |
| | (AT5G53040) | | |
| | AMINO ACID | | |
| | NP_200116.1 | | |
| SEQ ID NO: 19 | EASE PRO | EGG CELL-PREFERRED | PN |
| | | PROMOTER | |
| SEQ ID NO: 20 | AT-DD2 PRO | EGG CELL-PREFERRED | PN |
| | | PROMOTER | |
| SEQ ID NO: 21 | AT-RKD1 PRO | EGG CELL-PREFERRED | PN |
| SEQ ID NO: 22 | AT-RKD2 PRO | EGG CELL-PREFERRED | PN |
| SEQ ID NO: 23 | BA-BARNASE- | DNA ENCODING | PN |
| | INT | CYTOTOXIC | |
| | | POLYPEPTIDE | |
| SEQ ID NO: 24 | DAM | DNA ENCODING | PN |
| | METHYLASE | CYTOTOXIC | |
| | | POLYPEPTIDE | |
| SEQ ID NO: 25 | DMETH N-TERM | OLIGONUCLEOTIDE | PN |
| SEQ ID NO: 26 | INTE-N | OLIGONUCLEOTIDE | PN |
| SEQ ID NO: 27 | INTE-C | OLIGONUCLEOTIDE | PN |
| SEQ ID NO: 28 | DMETH C-TERM | OLIGONUCLEOTIDE | PN |
| SEQ ID NO: 29 | ADP | DNA ENCODING | PN |
| | RIBOSYLASE | CTYOTOXIC | |
| | | POLYPEPTIDE | |
| SEQ ID NO: 30 | FEM2 | EMBRYO SAC- | PN |
| | | PREFERRED | |
| | | PROMOTER | |
| SEQ ID NO: 31 | ATRKD5 | CDNA OF RKD | PN |
| | AT4G35590; DNA; | POLYPEPTIDE | |
| | ARABIDOPSIS | | |
| | THALIANA | | |
| SEQ ID NO: 32 | AT- | RKD POLYPEPTIDE | PP |
| | RKD5; PRT; ARABIDOPSIS | | |
| | THALIANA | | |
| SEQ ID NO: 33 | AT1G24540 | OVULE TISSUE- | PN |
| | AT-CP450-1 PRO | PREFERRED | |
| | | PROMOTER | |
| SEQ ID NO: 34 | ZMDD45PRO; | PROMOTER | PN |
| | DNA; ZEA MAYS | | |
| SEQ ID NO: 35 | PCO659480 | OLIGONUCLEOTIDE | PN |
| | 5PRIMELONG; | | |
| | DNA; ZEA MAYS | | |
| SEQ ID NO: 36 | PCO659480 | OLIGONUCLEOTIDE | PN |
| | 3PRIMELONG; | | |
| | DNA; ZEA MAYS | | |
| SEQ ID NO: 37 | ZSGREEN5PRIME; | OLIGONUCLEOTIDE | PN |
| | DNA; | | |
| | ZOANTHUS SP | | |
| SEQ ID NO: 38 | ZSGREEN3PRIME; | OLIGONUCLEOTIDE | PN |
| | DNA; | | |
| | ZOANTHUS SP | | |
| SEQ ID NO: 39 | CYAN1 5PRIME; | OLIGONUCLEOTIDE | PN |
| | DNA; ANEMONIA | | |
| | MAJANO | | |
| SEQ ID NO: 40 | CYAN1 3PRIME; | OLIGONUCLEOTIDE | PN |
| | DNA; ANEMONIA | | |
| | MAJANO | | |
| SEQ ID NO: 41 | AT-DD1 PRO; | PROMOTER | PN |
| | DNA; | | |
| | ARABIDOPSIS | | |
| | THALIANA | | |
| SEQ ID NO: 42 | AT-DD31 PRO; | PROMOTER | PN |
| | DNA; | | |
| | ARABIDOPSIS | | |
| | THALIANA | | |
| SEQ ID NO: 43 | AT-DD65 PRO; | PROMOTER | PN |
| | DNA; | | |
| | ARABIDOPSIS | | |
| | THALIANA | | |
| SEQ ID NO: 44 | SORGHUM | PROMOTER-OVULE | PN |
| | BICOLOR OVULE | | |
| | SPECIFIC | | |
| | PROMOTER 1 | | |
| | (SB10G008120.1) | | |
| SEQ ID NO: 45 | PROMOTER | PROMOTER-OVULE | PN |
| | RICE OVULE | | |
| | CANDIDATE 1 | | |
| | (OS02G-51090) | | |
| SEQ ID NO: 46 | AT-RKD2 PRO | PROMOTER WITH | PN |
| | (AT1G74480) | PROPOSED TETOP | |
| | | SITES. OPTION 1 | |
| SEQ ID NO: 47 | AT-RKD2 PRO | PROMOTER WITH | PN |
| | (AT1G74480) | PROPOSED TETOP | |
| | | SITES. OPTION 2 | |
| SEQ ID NO: 48 | AT-RKD2 PRO | PROMOTER WITH | PN |
| | (AT1G74480) | PROPOSED TETOP | |
| | | SITES. OPTION 3 | |
| SEQ ID NO: 49 | BA-BASTAR; | CYTOTOXIC COGNATE | PN |
| | DNA; BACILLUS | REPRESSOR | |
| | AMYLOLIQUEFACIENS | | |
| SEQ ID NO: 50 | AT-RKD3 PRO; | PROMOTER | PN |
| | DNA; | | |
| | ARABIDOPSIS | | |
| | THALIANA | | |
| SEQ ID NO: 51 | AT-RKD4 PRO; | PROMOTER | PN |
| | DNA; | | |
| | ARABIDOPSIS | | |
| | THALIANA | | |
| SEQ ID NO: 52 | AT-RKD5 PRO; | PROMOTER | PN |
| | DNA; | | |
| | ARABIDOPSIS | | |
| | THALIANA | | |
| SEQ ID NO: 53 | AT-LAT52LP1 | PROMOTER | PN |
| | PRO; DNA; | | |
| | ARABIDOPSIS | | |
| | THALIANA | | |
| SEQ ID NO: 54 | AT-LAT52LP2 | PROMOTER | PN |
| | PRO; DNA; | | |
| | ARABIDOPSIS | | |
| | THALIANA | | |
| SEQ ID NO: 55 | AT-PPG1 PRO; | PROMOTER | PN |
| | DNA; | | |
| | ARABIDOPSIS | | |
| | THALIANA | | |
| SEQ ID NO: 56 | AT-PPG2 PRO; | PROMOTER | PN |
| | DNA; | | |
| | ARABIDOPSIS | | |
| | THALIANA | | |

### II. Sequences Encoding Cytotoxin Polypeptides

Methods and compositions are provided which prevent formation of a sexual embryo in a plant ovule and to use this state to identify and promote asexual embryo formation (parthenogenesis). The embryo cell-like state is produced in the ovule plant cell by increasing the expression of at least one polypeptide or regulatory RNA in an ovule plant cell lacking the maintainer transgene cassette. The polypeptide or regulatory RNA will be identified by way of the parthenogenesis screen.

As used herein, parthenogenesis screen refers to controlled expression of a class of proteins that genetically ablates the egg cell and an integrated system to maintain the embryoless population. DAM methylase proteins are functionally analogous to DNA methyltransferases. The structures of various DNA methyltransferase polypeptides are known and the DNA methyltransferase genes have been identified in a variety of prokaryotes, lower eukaryotes and higher plants including Escherichia coli, Proteus vulgaris, Arabidopsis thaliana, Zea mays and Otyza sativa. Various methodologies may be used to maintain the embryoless population with a cytotoxin, including antisense RNA, RNAi, artificial microRNA, cognate inhibitors, aptamers and cognate antibody expression. BARNASE proteins are functionally analogous to ribonucleases. The structures of various ribonuclease polypeptides are known, and the ribonuclease genes have been identified in a variety of prokaryotes and eukaryotes including Bacteria and plants. Various methods and compositions are provided which employ polynucleotides and polypeptides having ribonuclease activity. Such ribonuclease polynucleotides include those set forth in any one of SEQ ID NO: 23, 24, 25 and 28, the polypeptides they encode and biologically active variants and fragments thereof. Further provided are the active variant and fragments of thereof.

As used herein, “parthenogenic activity” comprises a polypeptide that regulates embryogenesis. As used herein, a polypeptide having “parthenogenicactivity” comprises an regulatory polypeptide or an active variant or fragment thereof that retains sufficient parthenogenic activity such that (i) said polypeptide has regulatory activity; (ii) said polypeptide when expressed at sufficient levels in an ovule plant cell alters the transcriptional state to an embryo cell-like state and/or (iii) said polypeptide when expressed in a host plant cell increases expression of a gene operably linked to an embryo cell promoter including, for example, an embryo cell-preferred promoter comprising At1g60530, At3g63320, At1g66610 or AT1g53930 or other embryo cell-preferred promoters disclosed elsewhere herein. Methods to assay for such activity are known. See, for example, Koszegi. et al., (2011) Plant Journal Accelerated article, doi:101111/j.1365-313x.2011.04592.x, herein incorporated by reference. Non-limiting examples of female gametophyte-specific marker genes which are expressed in an egg cell-like transcriptional state include, but are not limited to, female gametophyte specific expressed genes AT1G18770 (MYB98), AT1G26795 (Self incompatibility protein-related), AT2G20070 (unknown), at4g25530 (homoebox protein, fwa) and at5g40260 (nodulin mtN3 family protein). See, Koszegi, et al., (2011) Plant Journal Accelerated article, doi:101111/j.1365-313x.2011.04592.x.

As used herein, an “isolated” or “purified” polynucleotide or polypeptide or biologically active portion thereof, is substantially or essentially free from components that normally accompany or interact with the polynucleotide or polypeptide as found in its naturally occurring environment. Thus, an isolated or purified polynucleotide or polypeptide is substantially free of other cellular material or culture medium when produced by recombinant techniques or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5% or 1% (by dry weight) of contaminating protein. When the polypeptide of the disclosure or biologically active portion thereof is recombinantly produced, optimally culture medium represents less than about 30%, 20%, 10%, 5% or 1% (by dry weight) of chemical precursors or non-protein-of-interest chemicals.

As used herein, polynucleotide or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A polypeptide expressed in vitro or in vivo from a recombinant polynucleotide is an example of a recombinant polypeptide. Likewise, a polynucleotide sequence that does not appear in nature, for example, a variant of a naturally occurring gene is recombinant.

A “control” or “control plant” or “control plant cell” provides a reference point for measuring changes in phenotype of the subject plant or plant cell, and may be any suitable plant or plant cell. A control plant or plant cell may comprise, for example: (a) a wild-type or native plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell which is genetically identical to the subject plant or plant cell but which is not exposed to the same treatment (e.g., herbicide treatment) as the subject plant or plant cell or (e) the subject plant or plant cell itself, under conditions in which the gene of interest is not expressed.

A. Active Fragments and Variants of Cytotoxin Sequences

As discussed above, methods and compositions are provided which employ polynucleotides and polypeptides having cytotoxin activity. Fragments and variants of cytotoxin polynucleotides and polypeptides are also encompassed. By “fragment” is intended a portion of the polynucleotide or a portion of the amino acid sequence and hence protein encoded thereby. Fragments of a polynucleotide may encode protein fragments that retain cytotoxin activity. Thus, fragments of a nucleotide sequence may range from at least about 20 nucleotides, about 50 nucleotides, about 100 nucleotides and up to the full-length polynucleotide encoding the cytotoxin polypeptides.

A fragment of an cytotoxin polynucleotide that encodes a biologically active portion of a cytotoxin protein will encode at least 50, 75, 100, 150, 175, 200, 225, 250, 275, 300, 325, 350, 375, 400, 410, 415, 420, 425, 430, 435 or 440 contiguous amino acids or up to the total number of amino acids present in a full-length cytotoxin polypeptide.

Thus, a fragment of a cytotoxin polynucleotide may encode a biologically active portion of a cytotoxin polypeptide. A biologically active portion of a cytotoxin polypeptide can be prepared by isolating a portion of one of the cytotoxin polynucleotides, expressing the encoded portion of the cytotoxin polypeptides (e.g., by recombinant expression in vitro), and assessing the activity of the cytotoxin portion of the cytotoxin protein. Polynucleotides that are fragments of a cytotoxin nucleotide sequence comprise at least 16, 20, 50, 75, 100, 150, 200, 250, 300, 350, 400, 450, 500, 550, 600, 650, 700, 800, 900, 1,000, 1,100, 1,200, 1,300 or 1,400 contiguous nucleotides or up to the number of nucleotides present in a full-length cytotoxin polynucleotide disclosed herein.

“Variant” protein is intended to mean a protein derived from the protein by deletion (i.e., truncation at the 5′ and/or 3′ end) and/or a deletion or addition of one or more amino acids at one or more internal sites in the native protein and/or substitution of one or more amino acids at one or more sites in the native protein. Variant proteins encompassed are biologically active, that is they continue to possess the desired biological activity of the native protein, that is, have cytotoxin activity. Such variants may result from, for example, genetic polymorphism or from human manipulation.

“Variants” is intended to mean substantially similar sequences. For polynucleotides, a variant comprises a polynucleotide having a deletion (i.e., truncations) at the 5′ and/or 3′ end and/or a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the cytotoxin polypeptides. Naturally occurring variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis or gene synthesis but which still encode a cytotoxin polypeptide.

Biologically active variants of a cytotoxin polypeptide (and the polynucleotide encoding the same) will have at least about 70%. 75%, 80%, 85%, 86%, 87%, 88%, 89%, 90%, 91%, 92%, 93%, 94%, 95%, 96%, 97%, 98%, 99% or more sequence identity to any cytotoxin polypeptide, including the polypeptide encoded any one of SEQ ID NO: 23, 24, 25 and 28 as determined by sequence alignment programs and parameters described elsewhere herein.

Biologically active variants of a cytotoxin polynucleotide will have at least about 70%. 75%, 80%, 85%, 86%, 87%, 88%, 89%, 90%, 91%, 92%, 93%, 94%, 95%, 96%, 97%, 98%, 99% or more sequence identity to any polynucleotide encoding a cytotoxin polypeptide, including the polynucleotide of any one of SEQ ID NO: 23, 24, 25 and 28 as determined by sequence alignment programs and parameters described elsewhere herein.

The cytotoxin polypeptide and the active variants and fragments thereof may be altered in various ways including amino acid substitutions, deletions, truncations and insertions. Methods for such manipulations are generally known in the art. For example, amino acid sequence variants and fragments of the cytotoxin proteins can be prepared by mutations in the DNA. Methods for mutagenesis and polynucleotide alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff, et al., (1978) Atlas of Protein Sequence and Structure (Natl. Biomed. Res. Found. Washington, D.C.), herein incorporated by reference. Conservative substitutions, such as exchanging one amino acid with another having similar properties, may be optimal.

Obviously, the mutations that will be made in the DNA encoding the variant must not place the sequence out of reading frame and optimally will not create complementary regions that could produce secondary mRNA structure. See, EP Patent Application Publication Number 75,444.

Variant polynucleotides and proteins also encompass sequences and proteins derived from a mutagenic and recombinogenic procedure such as DNA shuffling. With such a procedure, one or more different RDK coding sequences can be manipulated to create a new cytotoxin polypeptide possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the cytotoxin sequences disclosed herein and other known cytotoxin genes to obtain a new gene coding for a protein with an improved property of interest, such as a decreased Kₘ in the case of an enzyme. Strategies for such DNA shuffling are known in the art. See, for example, Stemmer, (1994) Proc. Natl. Acad. Sci. USA 91:10747-10751; Stemmer, (1994) Nature 370:389-391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291 and U.S. Pat. Nos. 5,605,793 and 5,837,458.

### III. Embryo Sac-cell, Embryo, Seed and Pollen Specific Tissue-Preferred Promoters

In seed plants, the ovule is the structure that gives rise to and contains the female reproductive cells. Early in development, it consists of three parts: the integument forming its outer layer, the nucellus (or megasporangium) and the funiculus. The nucellus produces the megasporocyte which will undergo meiosis to form the megaspores during megasporogenesis. In the Polygonum-type of embryo sac development, three of the megaspores degrade and one becomes the functional megaspore. During megagametogenesis, the functional megaspore (in Polygonum-type embryo sacs) goes through three rounds of syncytial mitoses to become an eight-nucleate cell. Cellularization occurs during further development to produce a mature embryo sac which includes an egg, synergids, antipodals, and the central cell with two polar nuclei in the typical Polygonum-type of embryo sac development. In some species (Zea spp.), antipodals can further divide and become numerous. Thus, as used herein, the ovule is initially composed of unreduced tissue that gives rise to the haploid tissue of the female gametophyte. The female gametophyte further develops into the “mature egg sac”, comprised of four unique cell types: one egg cell, a central cell, two synergids and three or more antipodal cells.

Various types of promoters can be employed in the methods and compositions provided herein. Promoters can drive expression in a manner that is cell-type-preferred, cell-type-specific, tissue-preferred or tissue-specific. Examples of promoters under developmental control include promoters that preferentially initiate transcription in certain tissues, such as leaves, roots, seeds or ovules. Such promoters are referred to as “tissue preferred”. Promoters which initiate transcription only in certain tissue are referred to as “tissue specific”. A “cell type” preferred promoter primarily drives expression in certain cell types in one or more organs, for example, vascular cells in roots, leaves or ovules. An “inducible” or “repressible” promoter is a promoter which is under environmental control. Examples of environmental conditions that may affect transcription by inducible promoters include anaerobic conditions or the presence of light. Tissue specific, tissue preferred, cell type specific, cell type preferred and inducible promoters constitute the class of “non-constitutive” promoters. A “constitutive” promoter is a promoter which is active under most environmental conditions.

As used herein, an “ovule tissue-preferred promoter” comprises a promoter that is predominately active in at least one or all of the ovule tissues of the plant, including for example, the integuments, and, nucellus when compared to its level of expression when not operably linked to the ovule tissue-preferred promoter. Thus, while some level of expression of an operably linked heterologous nucleotide sequence may occur in other plant tissue types, expression occurs most abundantly in the ovule tissue.

In specific embodiments, an ovule tissue-preferred promoter is employed which is “active in at least one non-gametophyte tissue in a plant ovule”. Such a promoter will be active in a somatic unreduced cell of the plant ovule that is outside of the embryo sac. Such a promoter may be active only in non-gametophyte tissue of the ovule or, alternatively, the promoter can show activity in the gametophytic tissue in addition to at least one other ovule tissue/structure. Non-limiting examples of promoters capable of directing expression in this manner include, the Arabidopsis NUC1 promoter region as set forth in SEQ ID NO: 1 or 2; the Arabidopsis CYP86C1 promoter region as set forth in SEQ ID NO: 3 or 4; the Arabidopsis PPM1 promoter region as set forth in SEQ ID NO: 5; the Arabidopsis EXT promoter region is set forth in SEQ ID NO: 6; the Arabidopsis GILT1 promoter region as forth in SEQ ID NO: 7; the Arabidopsis TT2 promoter region as forth in SEQ ID NO: 8; the Arabidopsis SLV3 promoter region as forth in SEQ ID NO: 9 and the Arabidopsis promoter AT1G24540 (AT-CP450-1-PRO) as set forth in SEQ ID NO: 33 or active variants and fragments thereof. In specific embodiments, the promoter employed is an ovule-specific promoter.

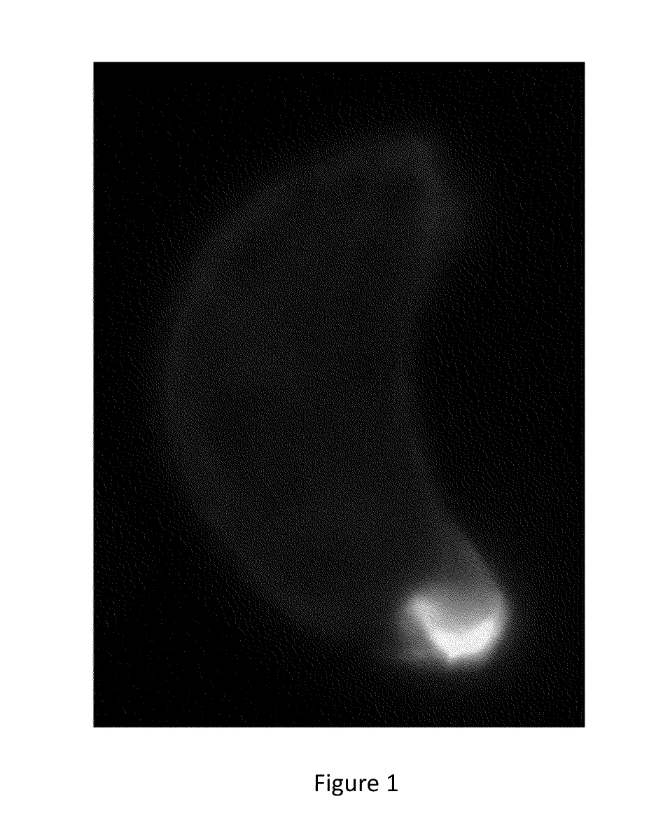

The promoter AT NUC1 (AT4G21620; GenBank: CP002687.1 (bps. 11496827-11495501), GENE ID: 828249; also known as F17L22.80; F17L22_{—}80; SEQ ID NO: 1 and 2) promoter demonstrates an expression pattern in the micropylar tip of the inner integument prior to fertilization. Expression further spreads chalazally through the inner integuments to surround the micropylar half of the embryo sac. Later in development, expression transitions from the micropylar inner integuments to the chalazal integuments. Expression appears present from several days before pollination to several days after pollination. At the heart-shaped embryo stage, expression is observed only at the integuments opposite the chalazal end. FIG. 1 provides the expression pattern of the AT NUC1 promoter. See also, US Patent Application Publication Number 2011/0107458A1, herein incorporated by reference.

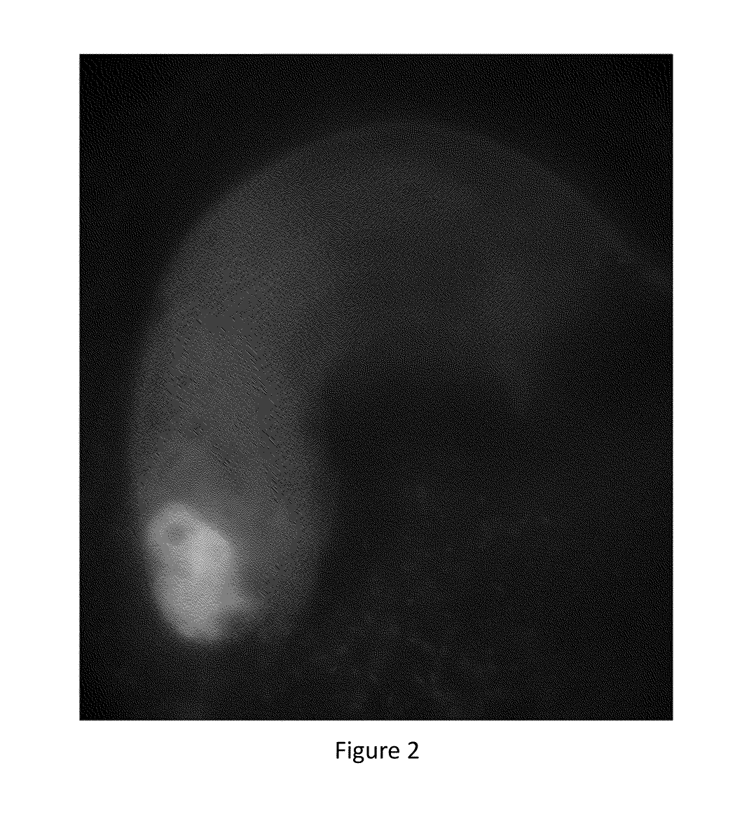

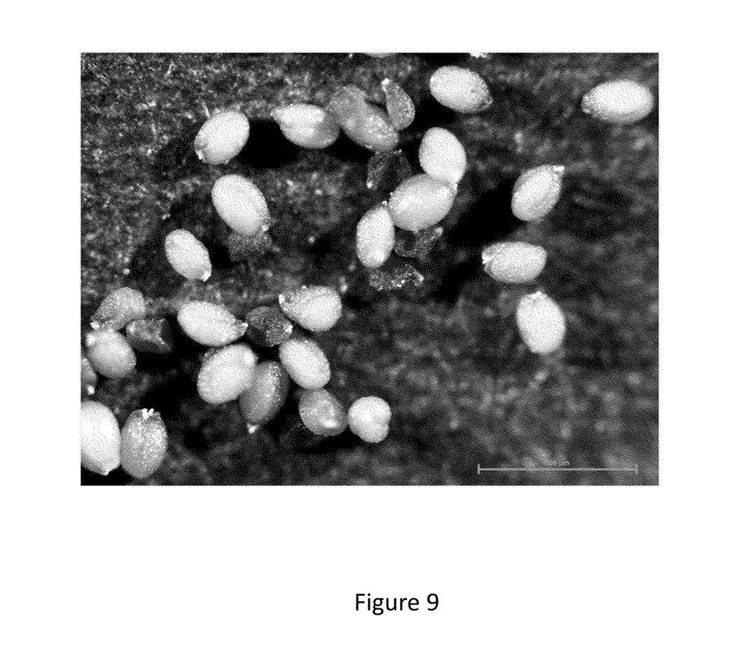

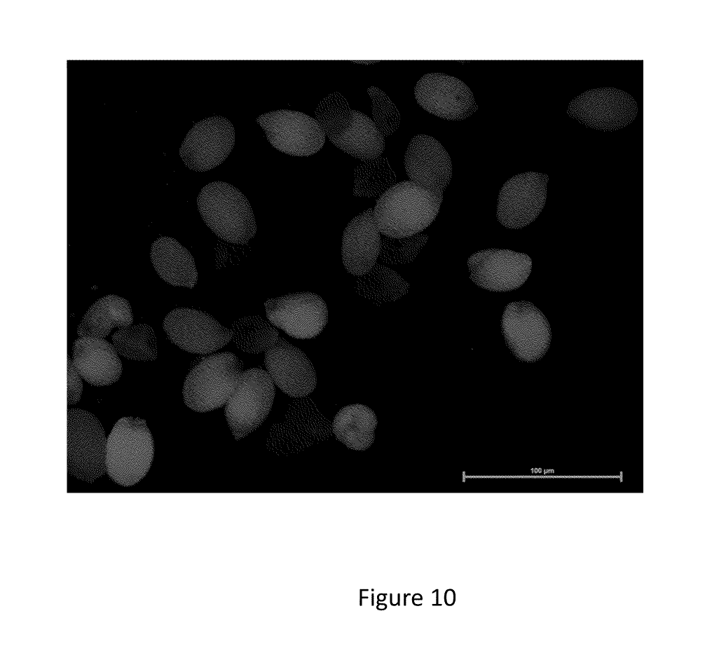

The promoter AT CYP86C1 (AT1G24540; GenBank: CP002684.1 (bps 8697732-8699750; other names: F21J9.20; SEQ ID NO: 3 or 4) displays an expression pattern in the micropylar tip of the inner integument prior to fertilization. Expression spreads chalazally through the endothelium (innermost layer of the inner integument) to surround the micropylar base of the embryo sac and expression then spreads chalazally through the entire endothelial layer. Expression appears present from several days before pollination to several days after pollination. FIGS. 2 through 10 provide the expression pattern of the CYP86C1 promoter.

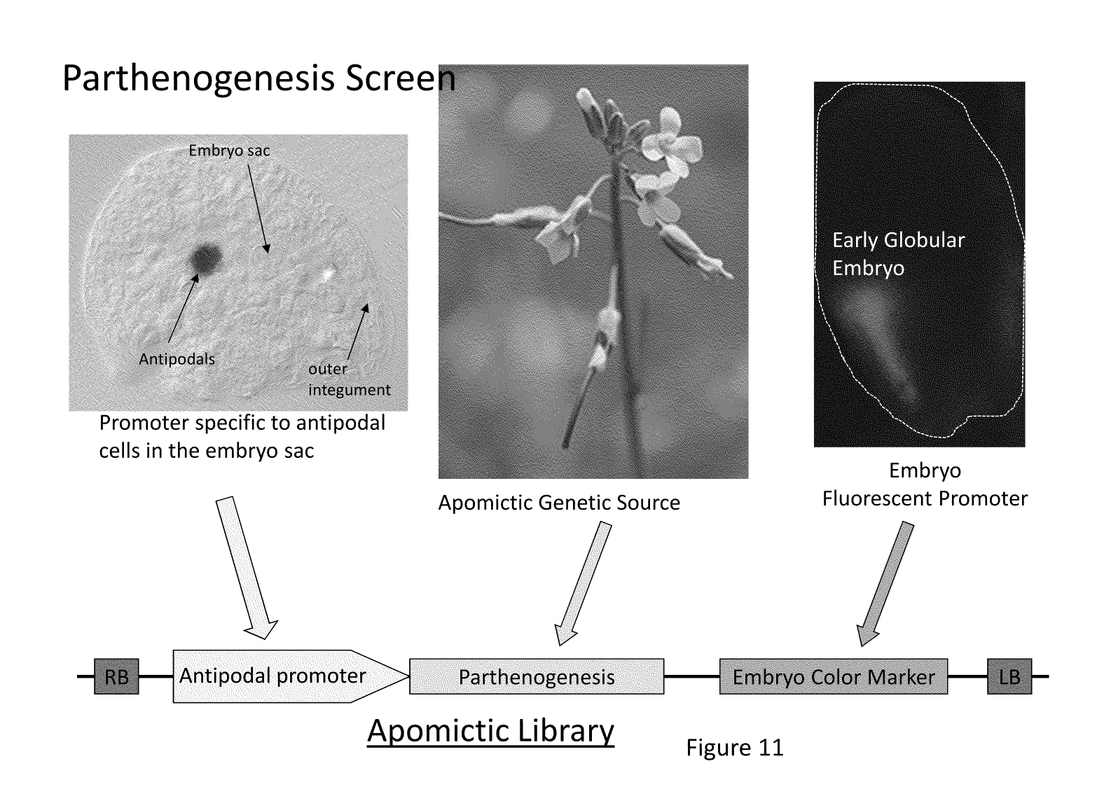

The promoter AT PPM1 (AT5G49180; GenBank: CP002688.1 (bps 19943368-19942879; other names: K21P3.5, K21P3_{—}5; SEQ ID NO: 5) demonstrates two types of expression patterns. First the AT PPM1 promoter demonstrates an expression pattern in the micropylar inner and outer integuments, but not the epidermal layer of the outer integument. The second type, of expression pattern is in the micropylar inner and outer integuments, as above, but expression extends chalazally through the inner and outer integuments (not epidermal layer) to surround the entire embryo sac, with the exception of the chalazal nucellus. No expression was observed within the embryo sac. The latter expression pattern was noted most commonly in early stages of ovule development. FIG. 11 provides the expression pattern of the AT PPM1 promoter. See also U.S. Pat. No. 7,179,904, U.S. Pat. No. 7,402,667, WO 2006/005023, WO 2006/066134, WO 2006/076099, WO 2007/075172, WO 2007/078286 and WO 2006/08102 and Louvet, et al., (2006) Planta 224:782, each of which is herein incorporated by reference.

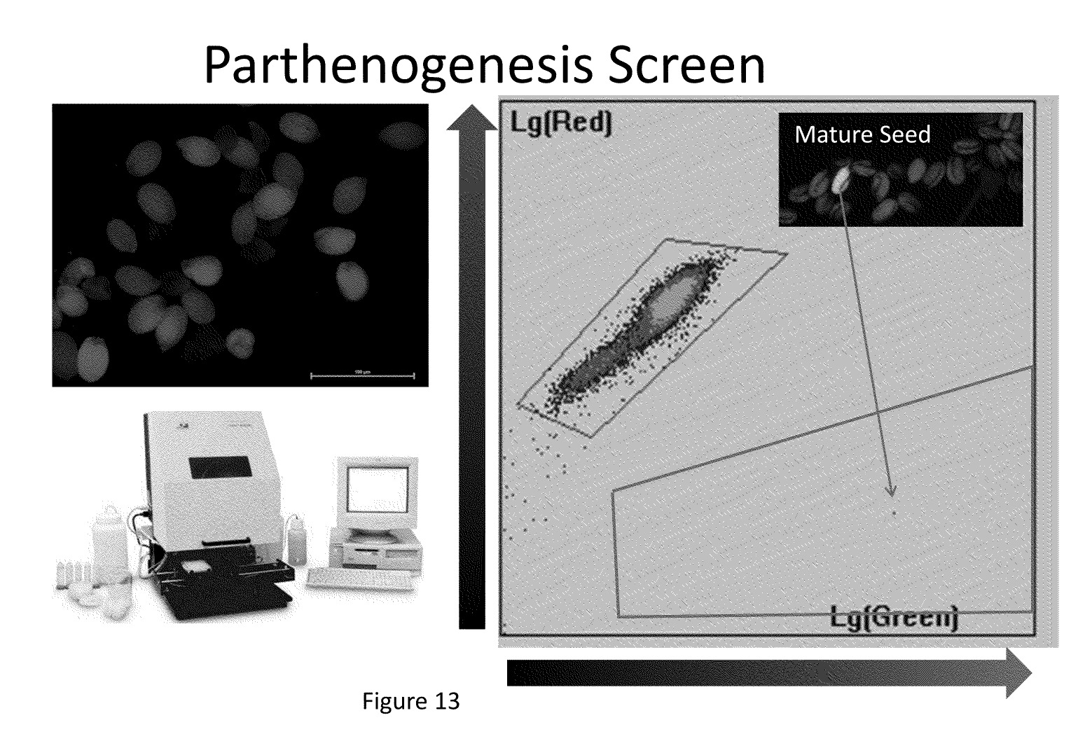

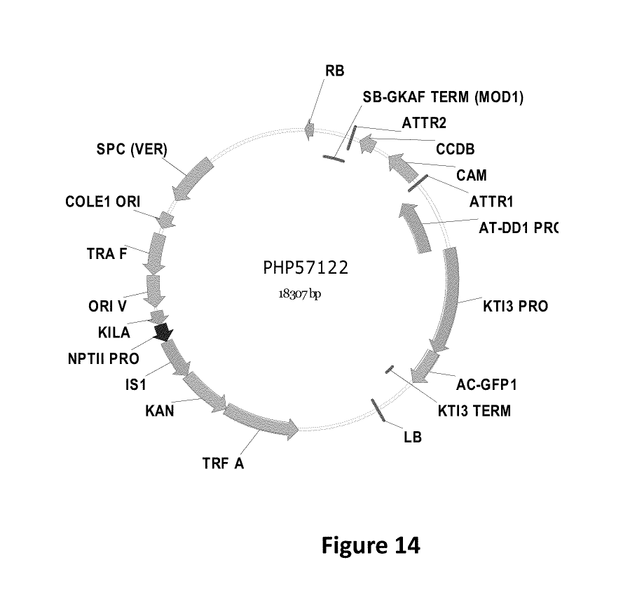

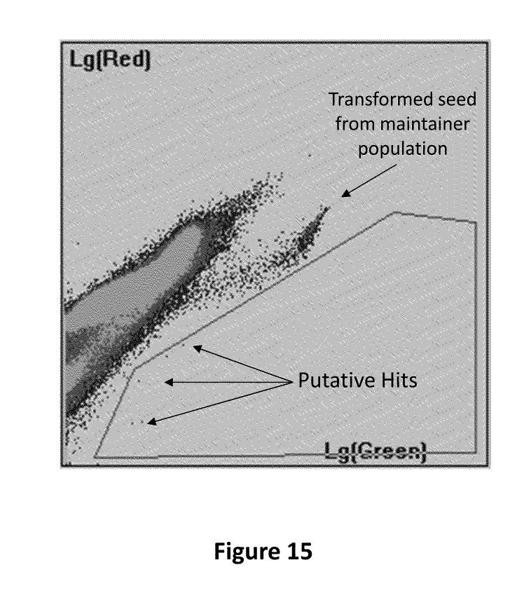

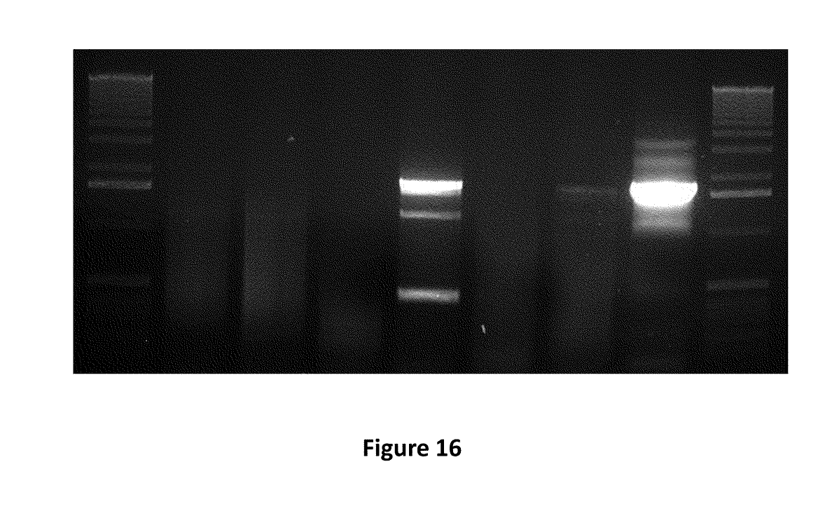

The promoter AT EXT (AT3G48580; Genbank CP002686.1, bps 18004981-18007235; also known as T8P19.90, XTH11, XYLOGLUCAN ENDO-TRANSGLUCOSYLASE/HYDROLASE 11; SEQ ID NO: 6) demonstrates an expression pattern in the inner integuments and innermost layer of the outer integument surrounding the micropylar end of the embryo sac. In addition, in one example, a single cell (innermost layer of outer integument) shows strong expression. The expression pattern for AT EXT is shown in FIG. 13.

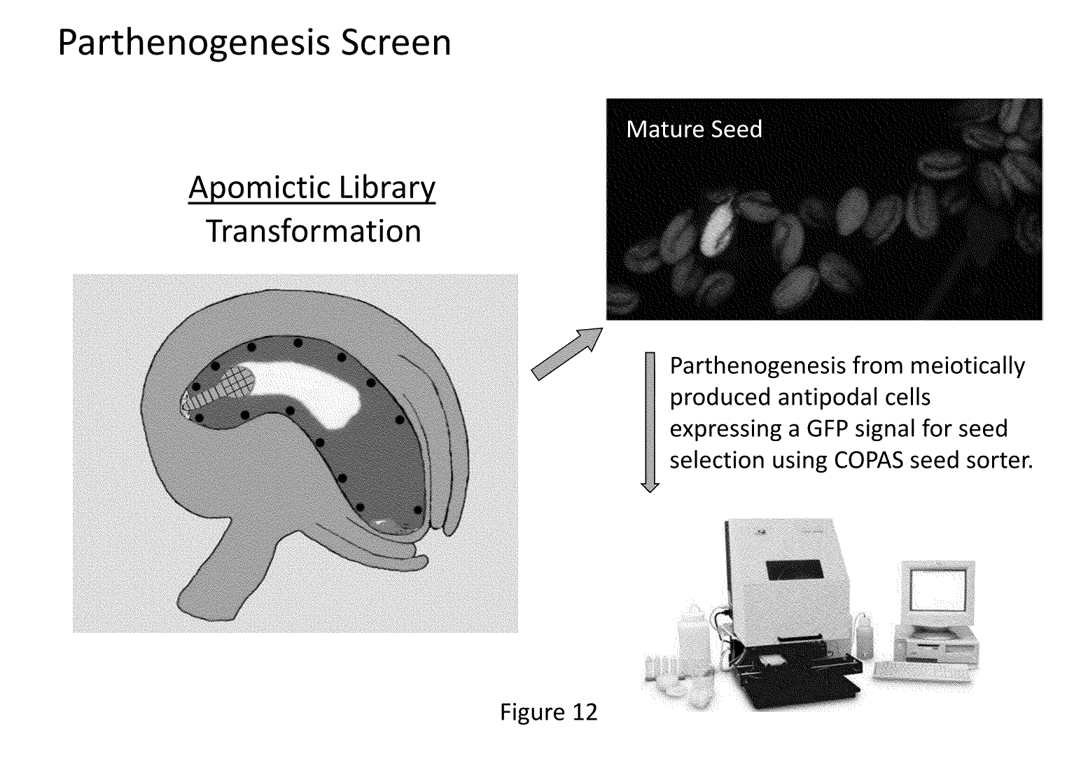

The promoter AT SVL3 (AT3G20520; GenBank Accession NM_{—}112944; also known as K10D20.6, SHV3-LIKE 3, SVL3; SEQ ID NO: 9) demonstrates an expression pattern that starts early during megagametogenesis. At the four nucleate megagametophyte stage, expression is initially strong in the micropylar inner and outer integuments spreading throughout the integuments of the entire ovule. Later in development, zygote stage, the endosperm and embryo also show expression. Thus, expression could be noted throughout the entire ovule with the exception of the funiculus. FIG. 12 provides the expression pattern for the AT-SVL3 promoter. Prior expression data is limited to expression in 6-week old siliques. See, Hayashi, et al., (2008) Plant Cell Physiol. 49:1522-1535, herein incorporated by reference.

Additional ovule tissue-preferred promoters that are active in at least one non-gametophyte tissue in a plant ovule include the promoter AT GILT1 (SEQ ID NO: 7; AT4G12890; Genbank CP002686.1 (bps 7545227- 7546409); other names: T20K18.240, T20K18-240. See also, U.S. Pat. No. 7,179,904, U.S. Pat. No. 7,402,667, U.S. Pat. No. 7,169,915, WO 2006/005023, WO 2006/066134, WO 2006/076099, WO 2007/075172, WO 2007/078286, WO 2006/081029 and WO 2002/016655 and Lovet, et al., (2006) Planta 224:782-791. Additional promoters include, AT TT2 (SEQ ID NO: 8; AT5G35550; GenBank Accession AJ299452; also known as Transparent Testa 2, ATMYB123, AT TT2, MOK9.18, MOK9_{—}18, MYB DOMAIN PROTEIN 123, MYB123, TT2). See also, WO 2006/031779; U.S. Pat. No. 6,972,197; WO 2000/055325 and Gonzalez, et al., (2009) Developmental Bio. 352 (2):412-421. Further promoters include the Arabidopsis promoter AT1G24540 as set forth in SEQ ID NO: 33 or active variants and fragments thereof.

Thus, the methods and compositions include isolated polynucleotides comprising the ovule tissue-preferred promoters disclosed above and also any ovule tissue-preferred promoter that is active in at least one non-gametophyte tissue in a plant ovule. Such sequences include the promoter nucleotide sequences set forth in SEQ ID NOS: 1, 2, 3, 4, 5, 6, 7, 8, 9 or 33. By “promoter” is intended a regulatory region of DNA usually comprising a TATA box capable of directing RNA polymerase II to initiate RNA synthesis at the appropriate transcription initiation site for a particular polynucleotide sequence. A promoter may additionally comprise other recognition sequences generally positioned upstream or 5′ to the TATA box, referred to as upstream promoter elements, which influence the transcription initiation rate. The promoter sequences disclosed herein regulate (i.e., activate) transcription from the promoter region.

It is recognized that additional domains can be added to the promoter sequences disclosed herein and thereby modulate the level of expression, the developmental timing of expression, or tissue type that expression occurs in. See particularly, Australian Patent Number AU-A-77751/94 and U.S. Pat. Nos. 5,466,785 and 5,635,618.

Fragments and variants of each of the ovule tissue-preferred promoter polynucleotides are further provided. Fragments of a promoter polynucleotide may retain biological activity and hence retain transcriptional regulatory activity. Thus, fragments of a promoter nucleotide sequence may range from at least about 20 nucleotides, about 50 nucleotides, about 100 nucleotides and up to the full-length polynucleotide of the disclosure. Thus, a fragment of an ovule tissue-preferred promoter polynucleotide may encode a biologically active portion of an ovule tissue-preferred promoter. A biologically active portion of an ovule tissue-preferred promoter polynucleotide can be prepared by isolating a portion of one of the ovule tissue-preferred promoter polynucleotides, and assessing the activity of the portion of the ovule tissue-preferred promoter. Polynucleotides that are fragments of an ovule tissue-preferred polynucleotide comprise at least 16, 20, 50, 75, 100, 150, 200, 250, 300, 350, 400, 450, 500, 550, 600, 650, 700, 800, 900, 1,000, 1,100, 1,200, 1,300, 1,400, 1,500, 1,600, 1,700, 1,800, 1,900, 2000, nucleotides or up to the number of nucleotides present in a full-length ovule tissue-preferred promoter polynucleotide disclosed herein.

For a promoter polynucleotide, a variant comprises a deletion and/or addition of one or more nucleotides at one or more internal sites within the native polynucleotide and/or a substitution of one or more nucleotides at one or more sites in the native polynucleotide. Generally, variants of a particular ovule tissue-preferred promoter will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Any of the promoter sequences employed herein can be modified to provide for a range of expression levels of the heterologous nucleotide sequence. Thus, less than the entire promoter region may be utilized and the ability to drive expression of the nucleotide sequence of interest retained. It is recognized that expression levels of the mRNA may be altered in different ways with deletions of portions of the promoter sequences. The mRNA expression levels may be decreased, or alternatively, expression may be increased as a result of promoter deletions if, for example, there is a negative regulatory element (for a repressor) that is removed during the truncation process. Generally, at least about 20 nucleotides of an isolated promoter sequence will be used to drive expression of a nucleotide sequence.

Variant polynucleotides also encompass sequences derived from a mutagenic and recombinogenic procedure such as DNA shuffling. With such a procedure, one or more different promoter sequences can be manipulated to create a new ovule tissue-preferred promoter possessing the desired properties. Strategies for such DNA shuffling are described elsewhere herein.

Methods are available in the art for determining if a promoter sequence retains the ability to regulate transcription in the desired temporal and spatial pattern. Such activity can be measured by Northern blot analysis. See, for example, Sambrook, et al., (1989) Molecular Cloning: A Laboratory Manual (2d ed., Cold Spring Harbor Laboratory Press, Plainview, N.Y.), herein incorporated by reference. Alternatively, biological activity of the promoter can be measured using assays specifically designed for measuring the activity and or level of the polypeptide being expressed from the promoter. Such assays are known in the art.

### IV. Expression Constructs

Methods and compositions are provided to increase the activity/level of a parthenogenic polypeptide in a plant ovule cell. In specific embodiments, such modulation of activity/level of the parthenogenic polypeptide promotes an egg cell-like state in an ovule plant cell. Such methods and compositions can employ an expression construct comprising a parthenogenic polypeptide or active variant or fragment thereof operably linked to an ovule tissue-preferred promoter, in particular an ovule tissue-preferred promoter that is active in at least one tissue in a plant ovule.

The expression cassette can include 5′ and 3′ regulatory sequences operably linked to the parthenogenic-encoding polynucleotide or an active variant or fragment thereof. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the parthenogenic encoding polynucleotide to be under the transcriptional regulation of the ovule tissue-preferred promoter. The expression cassette may additionally contain selectable marker genes.

The expression cassette will include in the 5′-3′ direction of transcription, an ovule tissue-preferred promoter or an active variant or fragment thereof, a parthenogenic encoding polynucleotide or active variant or fragment thereof, and a transcriptional and translational termination region (i.e., termination region) functional in the host cell (i.e., the plant). The regulatory regions (i.e., promoters, transcriptional regulatory regions and translational termination regions) and/or the parthenogenic encoding polynucleotides may be native/analogous to the host cell or to each other. Alternatively, the regulatory regions and/or the parthenogenic encoding polynucleotide or active fragments and variants thereof may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The termination region may be native with the transcriptional initiation region, may be native with the operably linked parthenogenic encoding polynucleotide or with the ovule tissue-preferred promoter sequences, may be native with the plant host, or may be derived from another source (i.e., foreign or heterologous) to the promoter, the parthenogenic encoding polynucleotide, the plant host, or any combination thereof. Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acids Res. 15:9627-9639.

Thus, expression constructs are provided comprising an ovule tissue-preferred promoter operably linked to a heterologous polynucleotide encoding a parthenogenic polypeptide, wherein the ovule tissue-preferred promoter is active in at least one tissue in a plant ovule. In still further embodiments, the polynucleotide encoding the parthenogenic polypeptide in the expression construct encodes a polypeptide as set forth in SEQ ID NO: 12, 14, 16 or 18; or it encodes a polypeptide having at least 80%, 85%, 90%, 91%, 92%, 93%, 94%, 95%, 96%, 97%, 98% or 99% sequence identity to the polypeptide set forth in SEQ ID NO: 12, 14, 16 or 18, wherein said active variant retains parthenogenic activity.

Moreover, the construct having the RDK encoding polynucleotide or active variant or fragment thereof can be operably linked to an ovule tissue-preferred promoter comprising the polynucleotide set forth in SEQ ID NO: 1, 2, 3, 4, 5, 6, 7, 8, 9 or 33; or, a polynucleotide having at least 80% A 85%, 90%, 91%, 92%, 93%, 94%, 95%, 96%, 97%, 98% or 99% sequence identity to SEQ ID NO: 1, 2, 3, 4, 5, 6, 7, 8, 9 or 33, wherein said polynucleotide retains the ability to direct expression of an operably linked polynucleotide in an ovule tissue-preferred.

In still further embodiments, the expression construct comprises: (i) the polynucleotide set forth in SEQ ID NO: 1 or 3 operably linked to the polynucleotide sequence encoding the polypeptide set forth in SEQ ID NO: 14 or (ii) the polynucleotide having at least 95% sequence identity to the sequence set forth in SEQ ID NO: 1 or 3, wherein said polynucleotide retains the ability to direct expression of an operably linked polynucleotide in an ovule tissue-preferred manner and said polynucleotide is operably linked to a polypeptide having at least 95% sequence identity to the polypeptide set forth in SEQ ID NO: 14, wherein said active variant retains parthenogenic activity.

Where appropriate, the polynucleotides may be optimized for increased expression in the transformed plant. That is, the polynucleotides can be synthesized using plant-preferred codons for improved expression. See, for example, Campbell and Gowri, (1990) Plant Physiol. 92:1-11 for a discussion of host-preferred codon usage. Methods are available in the art for synthesizing plant-preferred genes. See, for example, U.S. Pat. Nos. 5,380,831 and 5,436,391 and Murray, et al., (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein, et al., (1989) Proc. Natl. Acad. Sci. USA 86:6126-6130); potyvirus leaders, for example, TEV leader (Tobacco Etch Virus) (Gallie, et al., (1995) Gene 165 (2):233-238), MDMV leader (Maize Dwarf Mosaic Virus) (Johnson, et al., (1986) Virology 154:9-20) and human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) in Molecular Biology of RNA, ed. Cech (Liss, New York), pp. 237-256) and maize chlorotic mottle virus leader (MCMV) (Lommel, et al., (1991) Virology 81:382-385). See also, Della-Cioppa, et al., (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones and 2,4-dichlorophenoxyacetate (2,4-D). Additional selectable markers include phenotypic markers such as β-galactosidase and fluorescent proteins such as green fluorescent protein (GFP) (Su, et al., (2004) Biotechnol Bioeng 85:610-9 and Fetter, et al., (2004) Plant Cell 16:215-28), cyan florescent protein (CYP) (Bolte, et al., (2004) J. Cell Science 117:943-54 and Kato, et al., (2002) Plant Physiol 129:913-42) and yellow florescent protein (PhiYFP™ from Evrogen, see, Bolte, et al., (2004) J. Cell Science 117:943-54). For additional selectable markers, see generally, Yarranton, (1992) Curr. Opin. Biotech 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Aci. USA 86:5400-5404; Fuerst, et al., (1989) Proc. Natl. Acad. Sci. USA 86:2549-2553; Deuschle, et al., (1990) Science 248:480-483; Gossen, (1993) Ph.D. Thesis, University of Heidelberg; Reines, et al., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Baim, et al., (1991) Proc. Natl. Acad. Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob Agents Chemother 35:1591-1595; Kleinschnidt, et al., (1988) Biochemistry 27:1094-1104; Bonin, (1993) Ph.D. Thesis, University of Heidelberg; Gossen, et al., (1992) Proc. Natl. Acad. Sci. USA 89:5547-5551; Oliva, et al., (1992) Antimicrob Agents Chemother 36:913-919; Hlavka, et al., (1985) Handbook of Experimental Pharmacology, Vol. 78 (Springer-Verlag, Berlin); Gill, et al., (1988) Nature 334:721-724. Such disclosures are herein incorporated by reference. The above list of selectable marker genes is not meant to be limiting. Any selectable marker gene can be used.

It is further recognized that various expression constructs other than the parthenogenic expression construct are described herein. For example, expression constructs having sequences encoding marker sequences, cytotoxic polypeptides and embryo-inducing polypeptides are also described herein. One of skill will understand how to apply the language discussed above, to any expression construct.

### V. Sequences Encoding Embryo-Inducing Polypeptides

Methods and compositions are provided to increase the activity/level of a cytotoxin polypeptide in an ovule plant cell. In specific embodiments, such modulation of activity/level of the cytotoxin polypeptide promotes an egg cell-like state in an ovule plant cell. As discussed above, such methods and compositions employ an expression construct comprising a cytotoxin encoding polynucleotide operably linked to an ovule tissue-preferred promoter. Such methods and compositions can further be employed in combination with other sequences which encode embryo-inducing polypeptides.

As used herein, an “embryo-inducing polypeptide” comprises any sequence which when expressed in combination with the cytotoxin encoding polypeptide operably linked to an ovule tissue-preferred promoter further promotes the development of the egg cell-like state, including further promoting an egg cell-like transcription state, promoting the development of egg cell-like structures, promoting parthenogenesis and/or promoting partial parthenogenesis. Such embryo-inducing polyepetides can promote growth through triggering developmental programs.

Such embryo-inducing sequence include, but are not limited to, Somatic Embryogenesis receptor-like kinase (SERK) (Schmidt, et al., (1997) Development 124:2049-62), Wushel (WUS) (Zuo, et al., (2001) The Plant Journal 30:349-359, the family of LEC polypeptides including, Leafy Cotyledon1 (LEC1) (Lotan, et al., (1998) Cell 93:1195-1205) and Leafy Cotyledon2 (LEC2) (Stone, et al., (2001) PNAS 98:11806-11811), Baby Boom (BBM) (Boutilier, et al., (2002) Plant Cell 14:1737-1749) and agamous-like 15 (Harding, et al., (2003) Plant Physiol. 133:653-663), EMBRYOMAKER (EMK) (Tsuwamoto, et al., (2010) Plant Molecular Biology 73:481-492).

In specific embodiments, the embryo-inducing sequence is involved in organ development, initiation and/or development of the apical meristem. Such sequences include, for example, Wuschel (WUS) or active variants and fragments thereof. See U.S. Pat. Nos. 7,348,468 and 7,256,322 and United States Patent Application Publication Number 2007/0271628; Laux, et al., (1996) Development 122:87-96 and Mayer, et al., (1998) Cell 95:805-815, each of which are herein incorporated by reference. Modulation of WUS is expected to modulate plant and/or plant tissue phenotype including cell growth stimulation, organogenesis, and somatic embryogenesis. WUS may also be used to improve transformation via somatic embryogenesis. Expression of Arabidopsis WUS can induce stem cells in vegetative tissues, which can differentiate into somatic embryos (Zuo, et al., (2002) Plant J 30:349-359).

In yet another embodiment, a MYB118 gene (see, U.S. Pat. No. 7,148,402), MYB115 gene (se, Wang, et al., (2008) Cell Research 224-235), BABYBOOM gene (BBM; see, Boutilier, et al., (2002) Plant Cell 14:1737-1749), LEC and/or CLAVATA gene (see, for example, U.S. Pat. No. 7,179,963) is co-expressed with at least one expression cassette comprising at least one cytotoxin family member polypeptide.

In specific embodiments, the embryo-inducing sequence encodes a Leafy Cotyledon polypeptide (LEC) or an active variant or fragment thereof. The LEC family of transcription factors is involved in embryo maturation and functions in early developmental stages to maintain embryonic cell fate and have been shown to promote formation of embryo-like structures. See, for example, Lotan, et al., (1998) Cell 93:1195-1205; Braybrook, et al., (2008) Trends in Plant Science 13:624-630; Stone, (2001) PNAS 98:11806-11811; Gazzarrini, et al., (2004) Dev Cell 7:373-385; Gaj, et al., (2005) Planta 222:977-988; Wang, et al., (2007) Planta 226:773-783.

BABY BOOM (BBM or BNM3) or active variant and fragments thereof show similarity to the AP2/ERF family of transcription factors and is expressed preferentially in developing embryos and seeds. Ectopic expression of BBM in plants leads to the spontaneous formation of somatic embryos and cotyledon-like structures on seedlings. Ectopic BBM expression induced additional pleiotropic phenotypes, including neoplastic growth, hormone-free regeneration of explants, and alterations in leaf and flower morphology. BBM plays a role in promoting cell proliferation and morphogenesis during embryogenesis. See, Boutilier, et al., (2002) Plant Cell 14:1737-1749 and EP 1057891 (A1), both of which are herein incorporated by reference.

Other embryo-inducing polypeptides include members of the ARIADNE-subclass of RING-finger proteins. See, for example, Jackson, et al., (2000) Trends Cell Biol. 10:429-439 and Mladek, et al., (2003) Plant Physiol. 131:27-40, both of which are herein incorporated by reference. The ARIADNE proteins belong to a family of E3 ligases present in yeast, plants and animals and thought to be involved in the control of ubiquitin-dependent protein degradation (reviewed in Vierstra, (2003) Trends Plant Sci. 8:135-142). One member of the ARIADNE gene family is ARIADNE7 (ARI7). See, for example, Schallan, et al., (2010) The Plant Journal 62:773-784, herein incorporated by reference.

Biologically active variants of an embryo-inducing polypeptide (and the polynucleotide encoding the same) will have at least about 70%. 75%, 80%, 85%, 86%, 87%, 88%, 89%, 90%, 91%, 92%, 93%, 94%, 95%, 96%, 97%, 98%, 99% or more sequence identity to any embryo-inducing polypeptide, including but not limited to, the polypeptide of any one of SERK; Wushel (WUS); the family of LEC polypeptides; Baby Boom (BBM) and agamous-like 15, as determined by sequence alignment programs and parameters described elsewhere herein.

Thus, the cytotoxin encoding polynucleotides operably linked to the ovule tissue-preferred promoters can further be stacked with any combination of polynucleotide sequences of interest, particularly a sequence encoding an embryo-inducing polypeptide. Such stacking can occur within the same expression cassette or the two different sequences can be introduced into the plant separately. The desired stacked combinations can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. It is further recognized that polynucleotide sequences can be stacked at a desired genomic location using a site-specific recombination system. See, for example, WO 1999/25821, WO 1999/25854, WO 1999/25840, WO 1999/25855 and WO 1999/25853, all of which are herein incorporated by reference.

One of skill will recognize that the sequences encoding the embryo-inducing polypeptides can be placed into an expression cassette. Expression cassettes are discussed elsewhere herein. Any promoter of interest can be operably linked to the sequence encoding the embryo-inducing polypeptides, including for example, constitutive promoters, tissue-preferred promoters, tissue-specific promoters, ovule tissue-preferred promoters, an ovule tissue-preferred promoter that is active in at least one non-gametophyte tissue in a plant ovule, seed-preferred, embryo-preferred and/or endosperm preferred promoters. Many such promoters have been described elsewhere herein.

Non-limiting examples of constitutive promoters include, for example, the core promoter of the Rsyn7 promoter and other constitutive promoters disclosed in WO 1999/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142 and 6,177,611.

“Seed-preferred” promoters include both “seed-specific” promoters (those promoters active during seed development such as promoters of seed storage proteins) as well as “seed-germinating” promoters (those promoters active during seed germination). See, Thompson, et al., (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase) (see, WO 2000/11177 and U.S. Pat. No. 6,225,529, herein incorporated by reference). HV-NUC1 is a barley nucellus-specific promoter. Gamma-zein is an endosperm-specific promoter. Globulin 1 (Glb-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, Globulin 1, etc. See also, WO 2000/12733, where seed-preferred promoters from end1 and end2 genes are disclosed, herein incorporated by reference.

### VI. Sequence Encoding Cytotoxic Polypeptides

As discussed above, methods and compositions are provided to increase the activity/level of a cytotoxin polypeptide in an ovule plant cell. In specific embodiments, such modulation of activity/level of the cytotoxin polypeptide promotes an egg cell-like state in an ovule plant cell. Development of such a state (i.e., a transcriptional egg cell-like state or the development of embryo-like structures in tissues and substructures outside of the egg cell, including the formation of such structures in any tissues and substructures suitable for parthenogenesis) may be improved by further employing cytotoxic polypeptides which are expressed in a manner that allows for the targeted cell death or ablation of specific cell types of the embryo sac. In specific embodiments, at least the egg cell is ablated.

In specific embodiments, the egg cell in the plant ovule is specifically ablated and thereby the formation of the zygotic embryo is prevented. Since only the egg cell is ablated, fertilization of the central cell should be possible along with some degree of endosperm development. Prevention of the zygotic embryo allows for the synthetic apospory approach to self-reproducing plants or clonally reproducing plants. That is, the zygotic embryo is not formed, but an adventitious embryo is formed from non-reduced cells in the ovule through the expression of the RDK polypeptide as disclosed herein.

Thus, such methods which ablate the egg cell can be employed in combination with an expression construct comprising an ovule tissue-preferred promoter operably linked to a heterologous polynucleotide encoding a cytotoxin polypeptide, wherein the ovule tissue-preferred promoter is active in at least one tissue in a plant ovule and the ovule tissue-preferred promoter is active in an ovule cell of the plant.

Various cytotoxic polypeptides can be used for the targeted cell death or ablation of specific cell types of the embryo sac. In addition to the cytotoxin sequences outlined below, other possible cytotoxins include: alpha amylases, other nucleases; any method of gene silencing targeting genes that are required for egg cell development and/or expression of any protein or nucleic acid know to lead to cell death. Additional methods and compositions to ablate the egg cell, include, for example, an embryo-lethal mutation that is crossed into the plant can also be employed.

Such cytotoxic polypeptides include Barnase (a portmanteau of “BActerial” “RiboNucleASE”) which is a bacterial protein that consists of 110 amino acids and has ribonuclease activity. A non-limiting example of the barnase polypeptide is set forth in SEQ ID NO: 23. Note, INT refers to the addition of ST-LS1 INTRON2. Active fragments and variants thereof can further be employed, wherein said active fragments and variants retain cytotoxic activity in the cells in which they are expressed. Barnase is synthesized and secreted by the bacterium Bacillus amyloliquefaciens, but is lethal to the cell when expressed without its inhibitor barstar. The inhibitor binds to and occludes the ribonuclease active site, preventing barnase from damaging the cell's RNA after it has been synthesized, but before it has been secreted. See, for example, Buckle, et al., (1994) Biochemistry 33 (30):8878-8889; Serrano, et al., (1992) J. Mol. Biol. 224 (3):783-804; Serrano, et al., (1992). J. Mol. Biol. 224 (3):805-818; Matouschek, et al., (1992) J. Mol. Biol. 224 (3):819-835; Mossakowska, et al., (1989) Biochemistry 28 (9):3843-3850; Gils, et al., (2008) Plant Biotechnology Journal 6:226-235 and Kempe, et al., (2009) Plant Biotechnology Journal 7:283-297.

Additional cytotoxins that can be employed include, but are not limited to, a Dam Methylase as set forth in SEQ ID NO: 24 or an active variants or fragments thereof or the Dam Methylase Intein Split: DMETH N-term (SEQ ID NO:25); INTE-N (SEQ ID NO: 26); INTE-C (SEQ ID NO: 27); DMETH C-TERM (SEQ ID NO: 28) or active variants or fragments thereof or the ADP Ribosylase polypeptide (SEQ ID NO: 29) or active variants or fragments thereof.

Cell ablation to manipulate fertilization and/or seed development could include, for example, use of one or more cell-type-specific promoters disclosed herein. Thus, one of skill will recognize that the sequences encoding the cytotoxic polypeptides can be placed into an expression cassette. Expression cassettes are discussed elsewhere herein. Any promoter of interest can be operably linked to the sequence encoding the cytotoxic polypeptide, so long as the promoter directs expression of the cytotoxic polypeptide in cell type that one desires to ablate. Individual promoters would be particularly useful for cell ablation to prevent pollen tube attraction for fertilization (synergid ablation, DD31 or DD2); prevent sexual embryo formation (egg cell ablation, DD45) and/or prevent endosperm formation (central cell ablation, DD65). Such promoters include, for example, an embryo sac-preferred promoter or an embryo sac-specific promoter, including an egg cell-preferred promoter. Such egg-preferred promoters will not be active in the central cell or the endosperm and thereby these tissues are preserved when the egg cell-preferred promoter is operably linked to the sequence encoding the cytotoxic polypeptide. Such egg cell-preferred promoters include the Arabidopsis promoter (AT-DD45 PRO; Arabidopsis thaliana downregulated in dif1 (determinant infertile1; SEQ ID NO: 10; At2g21740 promoter) and active variants and fragments thereof. Analysis shows that this promoter is specific to the egg cell and zygote/early embryo, and is not expressed in any other cell types. When the AT-DD45 PRO is employed to express a cytotoxic polypeptide the egg cells in plant ovules will be specifically ablated. See, Steffen, et al., (2007) Plant J 51 (2):281-292. Using the DD45 promoter to express a toxin (e.g., BARNASE) would lead to egg cell ablation and prevent formation of the zygotic embryo. Since only the egg cell would be ablated, fertilization of the central cell should be possible along with some degree of endosperm development. Thus, such a construct when combined with the various methods disclosed herein can be used in the development of synthetic apospory.

Additional embryo sac-preferred promoters that can be used to express cytotoxic polypeptide include the antipodal cell-preferred promoter AT-DD1 PRO (SEQ ID NO: 40; downregulated with dif1 (determinant infertile1)1; At1g36340); a synergid cell-preferred promoter (AT-DD31 PRO; SEQ ID NO:42; downregulated with dif1 (determinant infertile1)1 31; At1g47470) and/or a central cell-preferred promoter (ATDD65PRO; SEQ ID NO: 43); downregulated with dif1 (determinant infertile1)1 65; At3g10890); Fem 2 (SEQ ID NO: 30; central-cell preferred/polar nuclei preferred) and active variant and fragments thereof. See also, U.S. Provisional Application Ser. No. ______, entitled Ovule Specific Promoters and Methods of Their Use, filed concurrently herewith and herein incorporated by reference in its entirety and Steffen, et al., (2007) The Plant Journal 51:281-292, herein incorporated by reference.

### VII. Variants and Fragments of Promoters

As discussed herein various promoters can be employed in the methods and compositions provided herein, including: promoters to express sequences encoding the embryo-inducing polypeptides and the sequences encoding the cytotoxic polypeptides. Fragments and variants of these promoter polynucleotides can be employed. Fragments of a promoter polynucleotide may retain biological activity and hence retain transcriptional regulatory activity in the desired tissue as the unmodified form. Thus, fragments of a promoter nucleotide sequence may range from at least about 20 nucleotides, about 50 nucleotides, about 100 nucleotides and up to the full-length promoter sequence. Thus, a fragment of a promoter polynucleotide may encode a biologically active portion of a promoter. A biologically active portion of a promoter polynucleotide can be prepared by isolating a portion of one of the promoter polynucleotides and assessing the activity of the portion of the promoter. Polynucleotides that are fragments of the polynucleotide comprise at least 16, 20, 50, 75, 100, 150, 200, 250, 300, 350, 400, 450, 500, 550, 600, 650, 700, 800, 900, 1,000, 1,100, 1,200, 1,300, 1,400, 1,500, 1,600, 1,700, 1,800, 1,900, 2000 nucleotides or up to the number of nucleotides present in a full-length promoter polynucleotide disclosed herein.

For a promoter polynucleotide, a variant comprises a deletion and/or addition of one or more nucleotides at one or more internal sites within the native polynucleotide and/or a substitution of one or more nucleotides at one or more sites in the native polynucleotide. Generally, variants of a particular promoter polynucleotide of the disclosure will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Methods are described elsewhere herein for determining if a promoter sequence retains the ability to regulate transcription in the desired temporal and spatial pattern.

It is recognized that to increase transcription levels, enhancers may be utilized in combination with the promoter disclosed herein. Enhancers are nucleotide sequences that act to increase the expression of a promoter region. Enhancers are known in the art and include the SV40 enhancer region, the 35S enhancer element and the like. Some enhancers are also known to alter normal promoter expression patterns, for example, by causing a promoter to be expressed constitutively when without the enhancer, the same promoter is expressed only in one specific tissue or a few specific tissues.

Modifications of the promoters disclosed herein can provide for a range of expression of the heterologous nucleotide sequence. Thus, they may be modified to be weak promoters or strong promoters. Generally, a “weak promoter” means a promoter that drives expression of a coding sequence at a low level. A “low level” of expression is intended to mean expression at levels of about 1/10,000 transcripts to about 1/100,000 transcripts to about 1/500,000 transcripts. Conversely, a strong promoter drives expression of a coding sequence at a high level or at about 1/10 transcripts to about 1/100 transcripts to about 1/1,000 transcripts.

### IIX. Plants and Methods of Making

The methods disclosed herein involve introducing a polypeptide or polynucleotide into a plant. “Introducing” is intended to mean presenting to the plant the polynucleotide or polypeptide in such a manner that the sequence gains access to the interior of a cell of the plant. The methods disclosed herein do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway, et al., (1986) Biotechniques 4:320-334), electroporation (Riggs, et al., (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski, et al., (1984) EMBO J. 3:2717-2722) and ballistic particle acceleration (see, for example, U.S. Pat. No. 4,945,050; U.S. Pat. No. 5,879,918; U.S. Pat. Nos. 5,886,244 and 5,932,782; Tomes, et al., (1995) in Plant Cell, Tissue, and Organ Culture: Fundamental Methods, ed. Gamborg and Phillips, (Springer-Verlag, Berlin); McCabe, et al., (1988) Biotechnology 6:923-926) and Lec1 transformation (WO 2000/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, N.Y.), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens), all of which are herein incorporated by reference.

In specific embodiments, the various sequences employed in the methods and compositions disclosed herein (e.g., the cytotoxin polypeptides, the embryo-inducing sequences, the cytotoxic polypeptides, etc.) can be provided to a plant using a variety of transient transformation methods. Such transient transformation methods include, but are not limited to, the introduction of the various sequences employed in the methods and compositions disclosed herein (e.g., the cytotoxin polypeptides, the embryo-inducing sequences, the cytotoxic polypeptides, etc. or variants and fragments thereof) directly into the plant or the introduction of the transcript into the plant. Such methods include, for example, microinjection or particle bombardment. See, for example, Crossway, et al., (1986) Mol Gen. Genet. 202:179-185; Nomura, et al., (1986) Plant Sci. 44:53-58; Hepler, et al., (1994) Proc. Natl. Acad. Sci. 91: 2176-2180 and Hush, et al., (1994) The Journal of Cell Science 107:775-784, all of which are herein incorporated by reference.

Alternatively, the various sequences employed in the methods and compositions disclosed herein (e.g., the cytotoxin polypeptides, the embryo-inducing sequences, the cytotoxic polypeptides, etc.) can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use particles coated with polyethyleneimine (PEI; Sigma #P3143).

In other embodiments, the polynucleotide of the disclosure may be introduced into plants by contacting plants with a virus or viral nucleic acids. Generally, such methods involve incorporating a nucleotide construct of the disclosure within a viral DNA or RNA molecule. It is recognized that the various sequences employed in the methods and compositions disclosed herein (e.g., the cytotoxin polypeptides, the embryo-inducing sequences, the cytotoxic polypeptides, etc.) may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221, herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO 1999/25821, WO 1999/25854, WO 1999/25840, WO 1999/25855 and WO 1999/25853, all of which are herein incorporated by reference. Briefly, the polynucleotide of the disclosure can be contained in a transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

Additional methods for targeted mutagenesis in vivo are known. For example, a DNA sequence having the desired sequence alteration can be flanked by sequences homologous to the genomic target. One can then select or screen for a successful homologous recombination event. See, U.S. Pat. No. 5,527,695. Generally, such a vector construct is designed having two regions of homology to the genomic target which flank a polynucleotide having the desired sequence. Introduction of the vector into a plant cell will allow homologous recombination to occur and to produce an exchange of sequences between the homologous regions at the target site.

Such methods of homologous recombination can further be combined with agents that induce site-specific genomic double-stranded breaks in plant cells. Such double strand break agents can be engineered to produce the break at a targeted site and thereby enhance the homologous recombination events. See, for example, Puchta, et al., (1996) Proc Natl Aced Sci USA 93:5055-5060; US Patent Application Publication Number 2005/0172365A1; US Patent Application Publication Number 2006/0282914, WO 2005/028942; WO 2004/067736 published Aug. 12, 2004; U.S. Pat. No. 5,792,632; U.S. Pat. No. 6,610,545; Chevalier et al., (2002) Mol Cell 10:895-905; Chevalier et al., (2001) Nucleic Acids Res 29:3757-3774; Seligman et al., (2002) Nucleic Acids Res 30:3870-3879; US Patent Application Publication Number 2009/0133152 and WO 2005/049842, each of which is herein incorporated by reference in their entirety.

The cells that have been transformed may be grown into plants in accordance with conventional ways. See, for example, McCormick, et al., (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide of the disclosure, for example, an expression cassette of the disclosure, stably incorporated into their genome.

As used herein, the term plant includes plant cells, plant protoplasts, plant cell tissue cultures from which plants can be regenerated, plant calli, plant clumps and plant cells that are intact in plants or parts of plants such as embryos, pollen, ovules, seeds, leaves, flowers, branches, fruit, kernels, ears, cobs, husks, stalks, roots, root tips, anthers and the like. Grain is intended to mean the mature seed produced by commercial growers for purposes other than growing or reproducing the species. Progeny, variants and mutants of the regenerated plants are also included within the scope of the disclosure, provided that these parts comprise the introduced polynucleotides.

The methods and compositions disclosed herein may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis) and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima) and chrysanthemum.

Conifers that may be employed in practicing the present disclosure include, for example, pines such as loblolly pine (Pinus taeda), slash pine (Pinus elliotil), ponderosa pine (Pinus ponderosa), lodgepole pine (Pinus contorta) and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea) and cedars such as Western red cedar (Thuja plicata) and Alaska yellow-cedar (Chamaecyparis nootkatensis). In specific embodiments, plants of the present disclosure are crop plants (for example, corn, alfalfa, sunflower, Brassica sp., soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica sp., maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc

### IX. Various Methods of Use

Methods for promoting an egg cell-like state in an ovule plant cell are provided. Such methods comprise expressing an expression construct comprising an ovule tissue-preferred promoter operably linked to a heterologous polynucleotide encoding a cytotoxin polypeptide, wherein the ovule tissue-preferred promoter is active in at least one tissue in a plant ovule. Such methods promote an egg cell-like state in at least one ovule cell of the plant outside of the embryo sac. In specific embodiments, the methods disclosed herein provide for the “egg cell-like state” to progress into the creation of parthenogenesis or initiation of embryony.

The ability to stimulate organogenesis and/or somatic embryogenesis may be used to generate an apomictic plant. Apomixis has economic potential because it can cause any genotype, regardless of how heterozygous, to breed true. It is a reproductive process that bypasses female meiosis and syngamy to produce embryos genetically identical to the maternal parent. With apomictic reproduction, progeny of a specially adaptive or hybrid genotypes would maintain their genetic fidelity throughout repeated life cycles. In addition to fixing hybrid vigor, apomixis can make possible commercial hybrid production in crops where efficient male sterility or fertility restoration systems for producing hybrids are not available. Apomixis can make hybrid development more efficient. It also simplifies hybrid production and increases genetic diversity in plant species with good male sterility. Furthermore, apomixis may be advantageous under stress (drought, cold, high-salinity, etc.) conditions where pollination may be compromised.

In specific embodiments, the encoded cytotoxin polypeptide employed in the methods disclosed herein comprises a polypeptide as set forth in SEQ ID NO: 12, 14, 16 or 18 or an active variant or fragment thereof. In addition, the ovule tissue-preferred promoter can comprise the polynucleotide set forth in SEQ ID NO: 1, 2, 3, 4, 5, 6, 7, 8, 9 or 33 or an active variant or fragment thereof. In still further embodiments, the expression construct comprises the polynucleotide set forth in SEQ ID NO: 1 or 3 or an active variant thereof operably linked to the polynucleotide sequence encoding the polypeptide set forth in SEQ ID NO: 14 or an active variant or fragment thereof.

Additional sequences can be used in the methods to promote the formation of an egg-like state. For example, expression of a RDK polypeptide from an ovule tissue-preferred promoter can be combined with the expression of an embryo-inducing polypeptide. Such embryo-inducing polypeptides are discussed elsewhere herein and comprises a BBM, WUS, LEC, MYB115, MYB118 and/or ARI7 polypeptide or an active variant thereof. The sequences encoding such embryo inducing polypeptides can be operably linked to any promoter including for example, an ovule tissue-preferred promoter.

In still further embodiments, the cytotoxin polypeptide is expressed in combination with a second polynucleotide which when expressed will ablate at least one cell within the embryo sac. In non-limiting examples, the second expression construct comprises an embryo-sac specific promoter operably linked to a polynucleotide which when expressed will ablate at least one cell within the embryo sac. The embryo sac-preferred promoter can be an antipodal cell-preferred promoter, a synergid cell-preferred promoter, an egg cell-preferred promoter or a central cell-preferred promoter. While in other embodiments, the embryo sac-preferred promoter is an egg cell-preferred promoter and comprises the polynucleotide set forth in SEQ ID NO: 10 or an active variant of fragment thereof.

Various methods and compositions that can be used to detect an egg-cell like state, an egg cell-like transcriptional state, development of egg cell-like structures, parthenogenesis and initiation of embryony are discussed elsewhere herein. In this manner, an egg cell-like state can be assayed for in tissues of the plant ovule including any tissues and substructures suitable for parthenogenesis.

Further provided are methods for modulating the concentration and/or activity of the cytotoxin polypeptide or active variant thereof in at least one tissue in a plant ovule. In other embodiments, the modulation of the concentration and/or activity of the cytotoxin polypeptide occurs in an ovule cell. In general, concentration and/or activity is increased by at least 1%, 5%, 10%, 20%, 30%, 40%, 50%, 60%, 70%, 80% or 90% relative to a native control plant, plant part or cell. Modulation in the present disclosure may occur during and/or subsequent to growth of the plant to the desired stage of development.

In specific embodiments, the methods of modulation (i.e., increasing) the concentration and/or activity of the cytotoxin polypeptide or an active variant or fragment thereof comprises introducing into the plant or plant cell a polynucleotide encoding the cytotoxin polypeptide employed comprises a polypeptide as set forth in SEQ 12, 14, 16 or 18 or an active variant or fragment thereof. In other embodiments, the sequence encoding the cytotoxin polypeptide is operably linked to an ovule tissue-preferred promoter, which can comprise the polynucleotide set forth in SEQ ID NO: 1, 2, 3, 4, 5, 6, 7, 8, 9 or 33 or an active variant or fragment thereof. In still further embodiments, the expression construct employed to modulate the level of the cytotoxin polypeptide comprises the polynucleotide set forth in SEQ ID NO: 1 or 3 or an active variant thereof operably linked to a the polynucleotide sequence encoding the polypeptide set forth in SEQ ID NO: 14 or an active variant or fragment thereof.

### IX. Additional Methods of Use for Ovule Tissue-Preferred Promoter Sequences

The various ovule-tissue preferred promoter sequences disclosed herein, as well as variants and fragments thereof, are useful in the genetic manipulation of any plant when assembled with a DNA construct such that the promoter sequence is operably linked to a heterologous polynucleotide encoding a heterologous protein or an RNA of interest. In this manner, the nucleotide sequences of the ovule-tissue preferred promoter sequences are provided in expression cassettes along with heterologous polynucleotides for expression in the plant of interest.

Synthetic hybrid promoter regions are known in the art. Such regions comprise upstream promoter elements of one nucleotide sequence operably linked to the promoter element of another nucleotide sequence. In an embodiment of the disclosure, heterologous gene expression is controlled by a synthetic hybrid promoter comprising the ovule-tissue preferred promoter sequences disclosed herein, or a variant or fragment thereof, operably linked to upstream promoter element(s) from a heterologous promoter.

The ovule-tissue preferred promoter sequences and methods disclosed herein are useful in regulating expression of any heterologous nucleotide sequence in a host plant in order to vary the phenotype of a plant. Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics and commercial products. Genes of interest include, generally, those involved in oil, starch, carbohydrate or nutrient metabolism as well as those affecting kernel size, sucrose loading and the like.

### X. Sequence Identity

The following terms are used to describe the sequence relationships between two or more nucleic acids or polynucleotides: (a) “reference sequence”, (b) “comparison window”, (c) “sequence identity”, (d) “percentage of sequence identity” and (e) “substantial identity”.

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the algorithm of Smith, et al., (1981) Adv. Appl. Math. 2:482; the algorithm of Needleman and Wunsch, (1970) J. Mol. Biol. 48:443-453; the algorithm of Pearson and Lipman, (1988) Proc. Natl. Acad. Sci. 85:2444-2448; the algorithm of Karlin and Altschul, (1990) Proc. Natl. Acad. Sci. USA 872:264, modified as in Karlin and Altschul, (1993) Proc. Natl. Acad. Sci. USA 90:5873-5877, herein incorporated by reference in their entirety.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA and TFASTA in the GCG Wisconsin Genetics Software Package®, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244 (1988); Higgins, et al., (1989) CABIOS 5:151-153; Corpet, et al., (1988) Nucleic Acids Res. 16:10881-90; Huang, et al., (1992) CABIOS 8:155-65 and Pearson, et al., (1994) Meth. Mol. Biol. 24:307-331, herein incorporated by reference in their entirety. The ALIGN program is based on the algorithm of Myers and Miller, (1988) supra. A PAM120 weight residue table, a gap length penalty of 12 and a gap penalty of 4 can be used with the ALIGN program when comparing amino acid sequences. The BLAST programs of Altschul, et al., (1990) J. Mol. Biol. 215:403, herein incorporated by reference in its entirety, are based on the algorithm of Karlin and Altschul, (1990) supra. BLAST nucleotide searches can be performed with the BLASTN program, score=100, word length=12, to obtain nucleotide sequences homologous to a nucleotide sequence encoding a protein of the disclosure. BLAST protein searches can be performed with the BLASTX program, score=50, word length=3, to obtain amino acid sequences homologous to a protein or polypeptide of the disclosure. To obtain gapped alignments for comparison purposes, Gapped BLAST (in BLAST 2.0) can be utilized as described in Altschul, et al., (1997) Nucleic Acids Res. 25:3389, herein incorporated by reference in its entirety. Alternatively, PSI-BLAST (in BLAST 2.0) can be used to perform an iterated search that detects distant relationships between molecules. See, Altschul, et al., (1997) supra. When utilizing BLAST, Gapped BLAST, PSI-BLAST, the default parameters of the respective programs (e.g., BLASTN for nucleotide sequences, BLASTX for proteins) can be used. See, the web site for the National Center for Biotechnology Information on the World Wide Web at ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. As used herein, “equivalent program” is any sequence comparison program that, for any two sequences in question, generates an alignment having identical nucleotide or amino acid residue matches and an identical percent sequence identity when compared to the corresponding alignment generated by GAP Version 10.

The GAP program uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package® for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package® is BLOSUM62 (see, Henikoff and Henikoff, (1989) Proc. Natl. Acad. Sci. USA 89:10915, herein incorporated by reference in its entirety).

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, optimally at least 80%, more optimally at least 90% and most optimally at least 95%, compared to a reference sequence using an alignment program using standard parameters. One of skill in the art will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, 70%, 80%, 90% and at least 95%.

The following examples are offered by way of illustration and not by way of limitation.

### EXAMPLES

The embodiments are further defined in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of the embodiments, and without departing from the spirit and scope thereof, can make various changes and modifications of them to adapt to various usages and conditions. Thus, various modifications of the embodiments in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1

### Creation of the Apomictic Library

Embryo Sac specific promoter (AT DD2 promoter, AT DD31 promoter, AT DD1 promoter), AT DD65 promoter, EASE promoter, or any other embryo sac promoter incorporated by reference above)

Apomicitic Genetic Source—for example: developing ovules from Boechera holboellii or other apomictic Boechera sp., apomictic orchids, apomictic apples, Malus sp., apomictic Rubus sp., apomictic Citrus sp., Hieracium sp., Hypericum sp., Pennisetum sp. or other apomictic or non-apomictic plant species.

Early Globular Embryo—KTI3 PRO:AC-GFP1

Transformation and separation of adventitious embryo from maternal tissues expressing a GFP signal for seed selection using Union Biometrica COPAS (Complex Object Parameter Analyzer and Sorter) seed sorter, found on the world wide web at www.unionbio.com/copas/U.S. Pat. No. 6,657,713, Dec. 2, 2003 and U.S. Pat. No. 6,400,453, Jun. 4, 2002, Large Object Sorter: Fluid Controlled Machine for Selecting and Depositing Multicellular Organisms, U.S. Pat. No. 7,116,407, Oct. 3, 2006, System For Axial Pattern Analysis of Multicellular Organisms (Profiler), Canadian Patent Number 2,341,231, Oct. 21, 2003, Large Object Sorter: Fluid Controlled Machine for Selecting and Depositing Multicellular Organisms.

Other means for separation of seeds can be accomplished by methods including but not limited to: non-fluorescence seed markers (color, shape, size, surface properties). Other selection properties can be exploited such as positive or counter selection. For instance, positive selection PTUs could be employed in the screening construct to select for herbicide resistance (for example) after the removal of seed containing the maintainer cassette.

### Example 2

### EGS System Mutant Scheme

This approach utilizes a maternal embryo defective (embryo lethal) recessive mutation which is then maintained in an approach similar to that used in the Sterile Inbred Maintenance System (SIMS) or Seed Production Technology (see, U.S. Pat. Nos. 7,696,405, 7,915,398 and 7,790,951). A transgenic cassette is introduced which has three parts: a wild type allele to complement the embryo lethal mutation, a pollen ablation PTU to prevent transgene transmission through the pollen and a seed color marker to allow removal of a transgenic population from the seeds produced. Another embodiment uses negative counter selection in the maintainer construct wherein a negative selection is activated through an inducible expression system, a metabolic counter-selection chemical application or other means. The resultant population will be homozygous for the recessive mutant allele, but transgenically complemented. These plants should segregate 1:1 in the subsequent generation for viable transgenic seed, and non-transgenic, non-viable, embryo-less homozygous mutants.

### Schematically:

2 types of plants:

Maternal embryo defective (embryo lethal) mutant: ee

Wild type allele to complement in hemizygous state: E−

Plant is ee+E/pollen-ablation PTU/seed color marker (E is only transmitted through egg)
When selfed: Female gametes are 50% e (embryo lethal), and 50% eE (embryo viable)
Male gametes are 100% e (all pollen carrying E are ablated)
Seeds produced by these plants are 50% ee (embryo lethal)

50% eEe (normal embryo due to complementing E, colored seed)
1) Construct B, a wild-type complementing transgene/egg-cell antidote line
2) a pollen ablation transgene
   a. Multiple were demonstrated
      i. AT-LAT52LP1 PRO:BA-BARNASE-INT
      ii. AT-PPG1 PRO:BA-BARNASE-INT
      iii. AT-LAT52LP2 PRO:ADP RIBOSYLASE (
      iv. AT-LAT52LP1 PRO:DMETH (Dam methylase)
      v. AT-LAT52LP2 PRO:DMETH (Dam methylase)
      vi. AT- PPG1 PRO:DMETH (Dam methylase)
3) a seed color marker
   a. Several have been demonstrated in Arabidopsis and maize
      i. Arabidopsis: KTI3 PRO:AC-GFP1; KTI3 PRO:AM-CYAN; RD29A PRO:DS-RED EXPRESS; RD29A PRO:ZS-YELLOW.
4) (for self-reproducing plants) a parthenogenesis PTU
   a. Promoters have been listed
   b. AT-RKD2 is a CDS candidate
   c. Promoter driving cDNA library linked to KTI3:AC-GFP1 as an embryo reporter. This constitutes an “parthenogenesis library”
   d. Use the Union Biometrica COPAS (Complex Object Parameter Analyzer and Sorter) to identify GFP positive seeds
      i. COPAS simultaneously detects optical density, time-of-flight, RED-, Yellow-, and Green-fluorescence.
      ii. The screen involves searching through seed for DS-RED negative, GFP positive seeds indicating an adventitious embryo was formed.
         1. DS-RED negative indicates the EGS maintainer is absent, and hence the egg cell was ablated and sexual zygote prevented
         2. GFP positive indicates the parthenogenesis library is present.

### Example 3

### Embryogenesis Gain-of-function Screen (EGS)

Wild type Arabidopsis plants are transformed with a construct containing: pollen ablation, egg cell +, and seed color marker. Plants are then selfed to create a hemizygous transgenic population.

Hemizygous transgenic population of Arabidopsis plants are then transformed with a construct containing egg ablation.

Seed from viable plants is grown and resultant transformed Arabidopsis plants are hemizygous for the egg ablation construct. These plants are transformed with a construct from apomictic library containing somatic embryony and embryo color marker.

Further to describe this in more detail:

Construct A contains egg cell specific promoter: toxin gene

Construct B contains egg cell specific promoter: toxin antidote/pollen ablation PTU/seed color marker

When a plant comprising both Construct A and Construct B is selfed:

Female gametes are 100% A+B (because A− only are non-viable)

Male gametes are 100% A (because A+B pollen is ablated)

Resultant seed produced is
100% (A+A)A (homozygous for construct A, hemizygous for construct B)

Selfing this generation produces,
50% AA/B− (viable transgenic)
50% AA/−− (non-viable embryoless)

Resultant seeds sorted by COPAS produce approximately 50% EGS egg+ seed (viable transgenic), 50% non-fluorescent aborted seed (nonviable embryoless).

The required components are:
1) Construct A, a recessive embryo-lethal mutant/egg-cell ablation line
2) Construct B, a wild-type complementing transgene/egg-cell antidote line
3) a pollen ablation transgene
   a. Multiple were demonstrated
      i. AT-LAT52LP1 PRO:BA-BARNASE-INT
      ii. AT-PPG1 PRO:BA-BARNASE-INT
      iii. AT-LAT52LP2 PRO:ADP RIBOSYLASE (
      iv. AT-LAT52LP1 PRO:DMETH (Dam methylase)
      v. AT-LAT52LP2 PRO:DMETH (Dam methylase)
      vi. AT- PPG1 PRO:DMETH (Dam methylase)
4) a seed color marker
   a. Several have been demonstrated in Arabidopsis and maize
      i. Arabidopsis: KTI3 PRO:AC-GFP1; KTI3 PRO:AM-CYAN; RD29A PRO:DS-RED EXPRESS; RD29A PRO:ZS-YELLOW.
5) (for self-reproducing plants) a parthenogenesis PTU
   a. Promoters have been listed
   b. AT-RKD2 is a CDS candidate
   c. Promoter driving cDNA library linked to KTI3:AC-GFP1 as an embryo reporter. This constitutes an “parthenogenesis library”
   d. Use the Union Biometrica COPAS (Complex Object Parameter Analyzer and Sorter) to identify GFP positive seeds
      i. COPAS simultaneously detects optical density, time-of-flight, RED-, Yellow-, and Green-fluorescence.
      ii. The screen involves searching through seed for DS-RED negative, GFP positive seeds indicating an adventitious embryo was formed.
         1. DS-RED negative indicates the EGS maintainer is absent, and hence the egg cell was ablated and sexual zygote prevented
         2. GFP positive indicates the parthenogenesis library is present.

### Example 4

### Activity of the Expression Cassette Comprising the Egg Ablation Reporter AT-RKD1:Barnase-Triple label (AT-DD45:DsRed AT-DD31:ZsYellow AT-DD65:AmCyan) in EGS Maintainer Line

FIG. 1 is a fluorescent image of a fertilized Arabidopsis embryo sac with only remnants of the egg/zygote (red) and of the synergids (green). Breakdown remnants of green and red may appear yellow. Central cell appears healthy with 3-4 endosperm nuclei indicating that fertilization did occur

### Example 5

### Activity of the Expression Cassette comprising the Egg Ablation Reporter AT-RKD2:Barnase-Triple label (AT-DD45:DsRed AT-DD31:ZsYellow AT-DD65:AmCyan) in EGS Maintainer Line

FIGS. 2 through 8 depict several events from the same transformation construct.

FIG. 2 is a fluorescent image of a fertilized Arabidopsis embryo sac with a zygote (red) that is in the process of breaking down, losing integrity and appears to be “blebbing”. The persistent synergid (green) appears to be condensing and breaking down as well. Central cell appears healthy with several endosperm nuclei indicating that fertilization did occur.

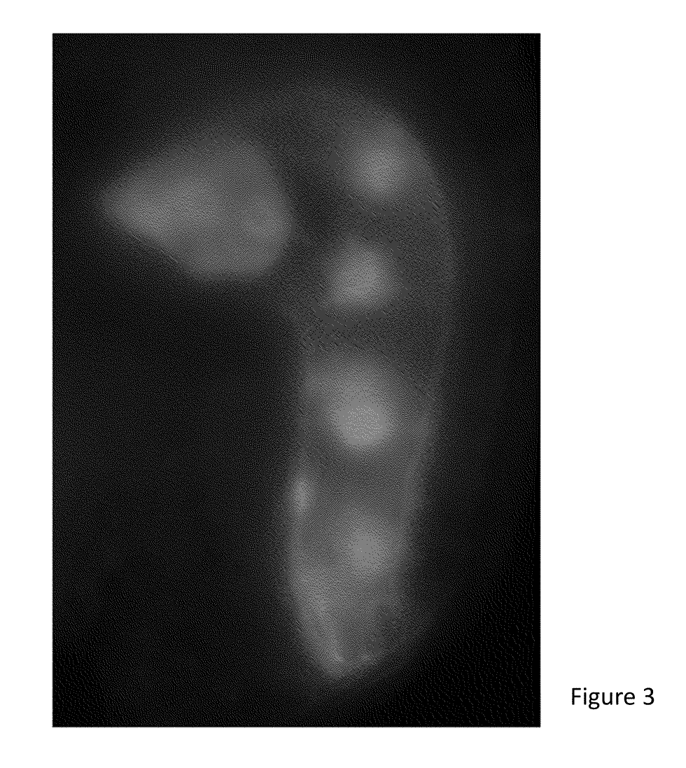

FIG. 3 is a fluorescent image of a fertilized Arabidopsis embryo sac showing 7-8 endosperm nuclei in a normal developing central cell. No sign of a zygote or embryo (red) nor any sign of a synergid (green) is present. The endosperm may be described as developing in the absence of an embryo.

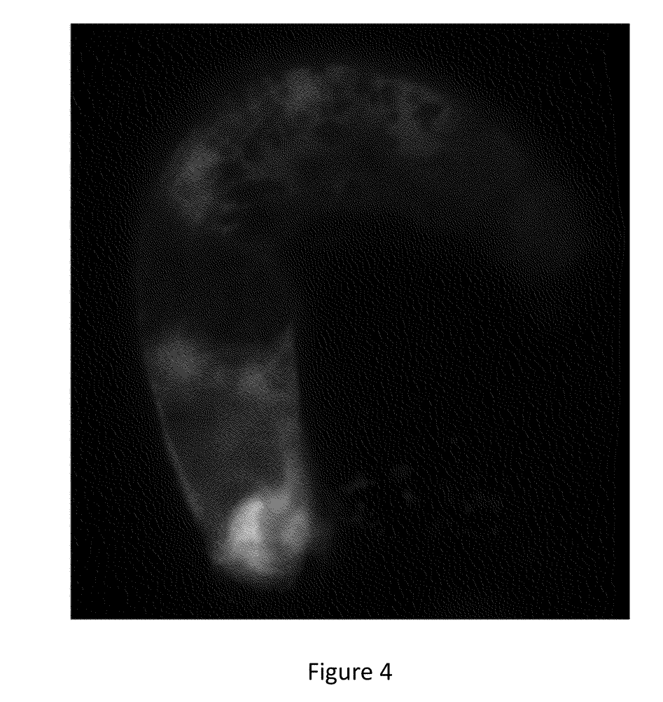

FIG. 4 is a fluorescent image of a fertilized Arabidopsis embryo sac with a remnant of the zygote (red) and the persistent synergid (green), where both appear to be condensing and breaking down. Central cell appears to be unhealthy and in the early stages of breaking down as is indicated by the increased vacuolation of the central cell.

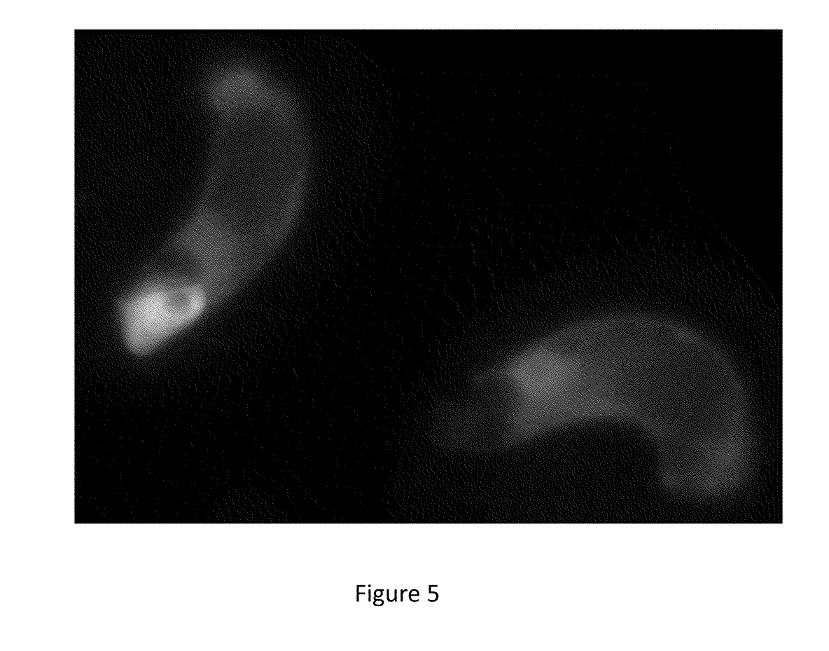

FIG. 5 is a fluorescent image of 2 unfertilized Arabidopsis embryo sacs just prior to fertilization. The embryo sac at left has a central cell (cyan) with the 2 endosperm nuclei and 2 synergids (yellow), but is lacking an egg (red). The embryo sac at right has a central cell (cyan) with the single primary endosperm nucleus, but is lacking the synergids (yellow) and the egg (red).

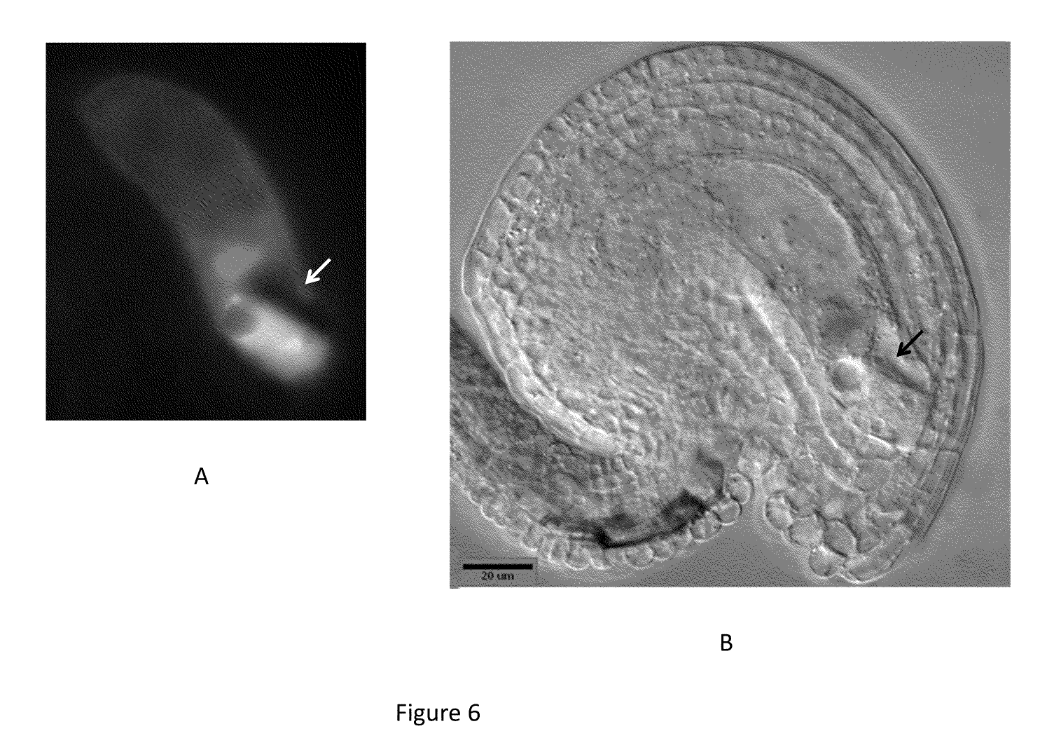

FIG. 6 is a fluorescent and differential interference contrast (DIC) fluorescent overlay image of a fertilized Arabidopsis embryo sac. The central cell (cyan) has the single endosperm nucleus and 1 synergid (yellow), but is lacking an egg (arrow).

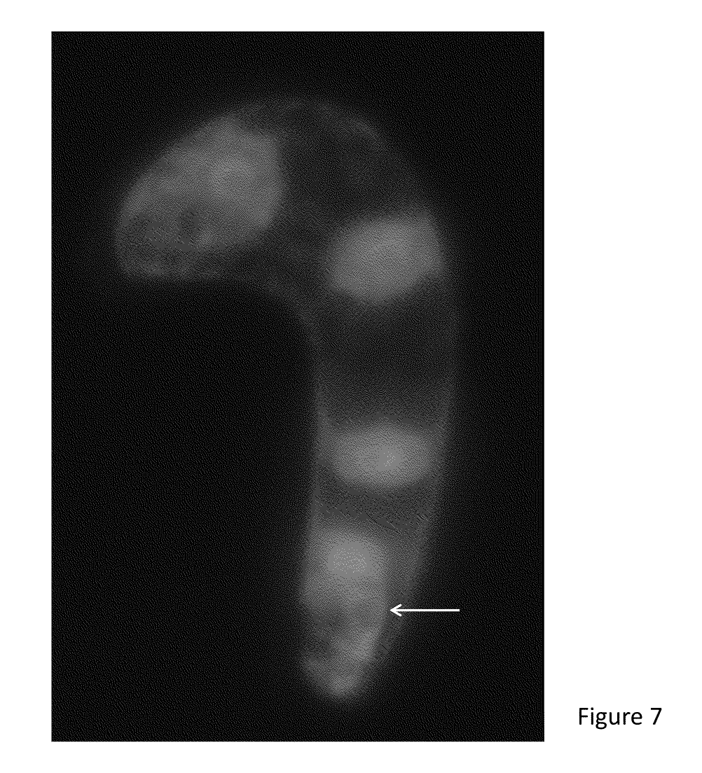

FIG. 7 is a fluorescent image of a fertilized Arabidopsis embryo sac with 4 endosperm nuclei in a normal developing central cell. Only a very weak red fluorescent signal (arrow) indicative of a remnant of the embryo or zygote is present. The persistent synergid (green) is breaking down. The endosperm is developing in the absence of an embryo.

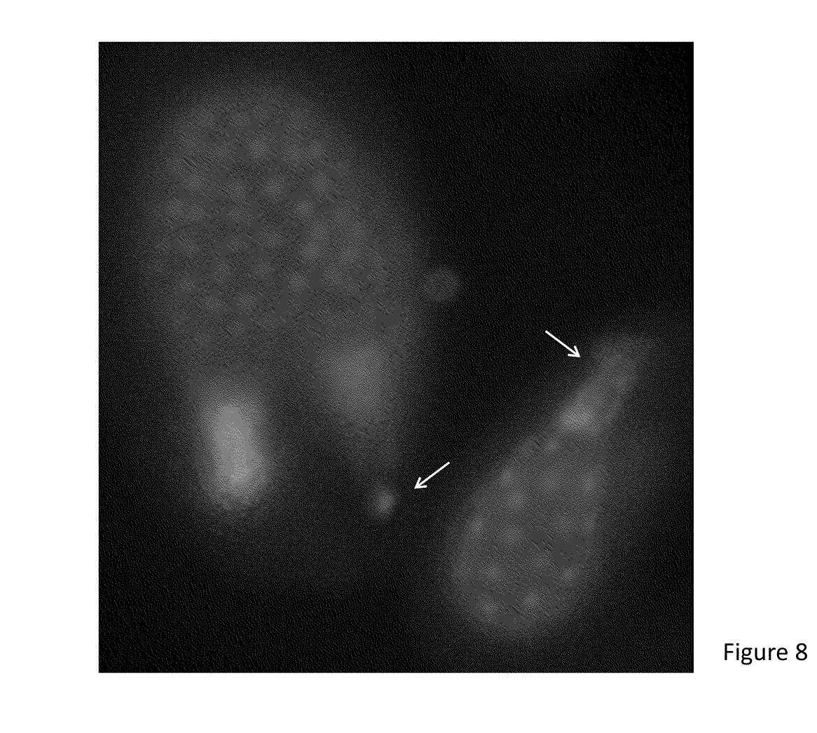

FIG. 8 is a fluorescent image of 2 Arabidopsis embryo sacs with well developed endosperm. The embryo sac at left has numerous endosperm nuclei in its central cell (cyan) and at its micropylar end (arrow) is a remnant of the embryo or zygote (red). Under normal conditions this embryo should be much more fully developed, at the heart-shaped stage. The smaller embryo sac at right has numerous endosperm nuclei (cyan) but is lacking an embryo (arrow). Synergids are naturally degraded by this late stage.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

That which is claimed:

1. A method for producing large seed population that lacks reproductively competent sexually derived embryos, comprising,
a) Transforming a first plant with a first transgenic cassette comprising 3 parts:
i) An egg-cell expressing promoter driving the cognate antidote,
ii) A pollen ablation PTU
iii) A seed selection marker;
b) Transforming said plant with a second expression cassette, wherein said second expression cassette comprises a nucleic acid molecule encoding a toxin gene expressed via an egg-cell specific promoter, creating a dominant hemizygous embryo-less phenotype, to generate a plant population which is homozygous for the toxin but hemizygous for fertility; and
c) Growing the seed from the plants transformed with the second cassette to generate plants having 50% of its seeds viable and transgenic for the first and second expression cassette.

2. The method of claim 1, wherein said first expression cassette comprises a nucleic acid molecule encoding a cognate antidote, wherein said antidote is selected from the group consisting of: SEQ ID NO: 49 or an active variant or fragment thereof.

3. The method of claim 2, wherein said first expression cassette comprises a nucleic acid molecule encoding a seed color marker comprising a fluorophore is selected from the group consisting of: DS-RED, ZS-GREEN, ZS-YELLOW, AC-GFP, AM-CYAN, and AM-CYAN1, AC-GFP, eGFP, eCFP. eYFP, eBFP, a “fruit” fluorescent protein (UC system); tagRFP, tagBFP, mKate, mKate2, tagYFP, tagCFP, tagGFP, TurboGFP2, TurboYFP, TurboRFP, TurboFP602, TurboFP635, TurboFP650, NirFP or Cerulean.

4. The method of claim 3, wherein said first expression cassette comprises a pollen ablation plant transcriptional unit (PTU), with a promoter selected from the group comprising SEQ ID NOS: 53, 54, 55 and 56.

5. The method of any one of claims 1-4, wherein said first expression cassette further comprises a tissue-specific promoter operably linked to said nucleic acid molecule encoding a cognate antidote polypeptide.

6. The method of claim 5, wherein said tissue-specific promoter is an egg-cell tissue specific promoter.

7. The method of claim 6, wherein said egg-cell tissue specific promoter is selected from the group comprising SEQ ID NOS: 1-9, 11, 13, 15, 17, 19-21, 31 and 33.

8. The method of any one of claims 1-7, wherein said second cassette further comprises an egg-cell tissue specific promoter operably linked to a toxin gene.

9. The method of claim 8, wherein said second cassette promoter is selected from the group consisting of: SEQ ID NOS: 53, 54, 55 and 56.

10. The method of any one of claims 1-9, wherein said plant is transformed with an parthenogenesis PTU and the toxin and/or antidote complementary constructs are removed to allow production of self-reproducing plants.

11. The method of claim 10, where the plant is a dicot plant.

12. The method of claim 11, wherein said dicot is Brassica sp., sunflower, cotton, canola, safflower, tobacco, Arabidopsis sp. or alfalfa.

13. The method of any one of claims 1-12, wherein said self-reproducing plant is soybean.

14. The method of any one of claims 1-10, wherein said self-reproducing plant is a monocot plant.

15. The method of claim 14, wherein said monocot is maize, wheat, rice, barley, sorghum or rye.

16. A self-reproducing plant produced by the method of any one of claims 1-15.

17. A seed of the self-reproducing plant of claim 16.
